(19) United States
(12) Patent Application Publication
(10) Pub. No.: US 2026/0048198 A1
(43) Pub. Date: Feb. 19, 2026

(54) WEARABLE DEVICE FOR PATIENT MONITORING AND DRUG ADMINISTRATION

(71) Applicant: Willow Laboratories, Inc., Irvine, CA (US)

(72) Inventors: Hung The Vo, Fountain Valley, CA (US); Sai Kong Frank Lee, Irvine, CA (US); Richard Velasco, Irvine, CA (US); Tran Minh Tuan, Irvine, CA (US)

(21) Appl. No.: 19/297,513

(22) Filed: Aug. 12, 2025

Related U.S. Application Data
(60) Provisional application No. 63/682,899, filed on Aug. 14, 2024.
(60) Provisional application No. 63/697,379, filed on Sep. 20, 2024.

(51) Int. Cl. A61M 5/172; A61M 5/14; A61M 5/142

(52) U.S. Cl. CPC A61M 5/172; A61M 5/1413; A61M 5/1422; A61M 2005/14252; A61M 2205/0266; A61M 2205/3317; A61M 2205/3331; A61M 2205/3368

(57) ABSTRACT

A modular disease management system may provide medication management for a patient. The system may include a base configured to at least partially couple to a tissue site of a patient. The system may further include a medication bladder coupled to the base and configured to maintain medication. The system may further include a medication pump coupled to the medication bladder, comprising a conduit and muscle wire-actuated plungers, wherein the medication pump is configured to cause the medication to flow from the medication bladder through the conduit. The system may further include a cannula insertion device coupled to the medication pump via the conduit, wherein the cannula insertion device comprises a cannula, a spring, and a triggering component, wherein the cannula insertion device is configured to insert the cannula into the tissue site of the patient by actuation of the spring released by the triggering component.

### FIELD OF THE DISCLOSURE

The general field of this disclosure is glucose disease management systems.

### BACKGROUND

Diabetes is a chronic disease that impacts many individuals, both adults and children. The management of diabetes may include the measurement of glucose within the interstitial space including blood and/or interstitial fluid of a patient and administration of insulin to the patient. A closed loop insulin administration system includes both a sensor to take glucose measurements from the interstitial space including blood and/or interstitial fluid of the patient and an insulin administration device which administers insulin to the patient based on the glucose measurements. Closed loop insulin administration systems allow individuals impacted by diabetes to go about daily life with much less worry about their insulin or glucose levels which can vastly improve a diabetic's quality of life.

### SUMMARY

Various aspects of systems, methods and devices within the scope of the appended claims each have several aspects, no single one of which is solely responsible for the desirable attributes described herein. Without limiting the scope of the appended claims, some prominent features are described herein.

In some aspects, the techniques described herein relate to a disease management system, including: a base configured to at least partially couple to a tissue site of a patient; a medication bladder coupled to the base and configured to store medication; a medication pump coupled to the medication bladder, including a conduit, wherein the medication pump is configured to cause the medication to flow from the medication bladder through the conduit; and a cannula insertion device coupled to the medication pump via the conduit, wherein the cannula insertion device includes a cannula, a spring, and a triggering component, wherein the cannula insertion device is configured to insert the cannula into the tissue site of the patient by actuation of the spring released by the triggering component.

In aspects, the present closure provides for a medication delivery pump system for delivering fluid medication to a user, the system including: a pump, the pump including: a first actuator; a second actuator, were each of the first actuator and second actuator can manipulate a medication volume passing through a medication flow path of the medication delivery pump system, each of the first actuator and second actuator including: a plunger; a disc spring, the disc spring opposing actuation of the plunger; a shape memory alloy (SMA) wire configured to drive movement of the plunger, wherein the positions of the plunger fall within an inclusive range between an actuated and an unactuated position in the medication flow path; and an electrical sensor configured to measure an electrical property of the SMA actuator; one or more environmental sensors; and a processor operably coupled to the electrical sensors, the SMA wires, and the one or more environmental sensors, the processor configured to execute computer readable instructions to: receive a first electrical property from the electrical sensor of the first actuator and a second electrical property from the electrical sensor of the second actuator; receive one or more signals from the one or more environmental sensors; and control the first actuator and the second actuator, based at least in part on the first electrical property, the second electrical property, and the one or more signals from the one or more environmental sensors, to drive the movement of the first actuator and the second actuator to positions within the medication flow path correlated with a target medication dispense volume.

In some examples, the one or more environmental sensors include at least one of a temperature sensor, a pressure sensor, or a humidity sensor. In some examples, the one or more environmental sensors include a temperature sensor, the temperature sensor positioned spaced from skin of a user when the medication delivery pump system is positioned on skin of a user. In some examples, the SMA wire includes a muscle wire. In some further examples, the muscle wire is configured to contract when electrical current is applied. In some further examples, the muscle wire includes one or more of nitinol or nickel titanium alloy.

In some examples, the pump includes: a third actuator configured to manipulate a medication volume passing through a medication flow path of the medication delivery pump system, the third actuator comprising: a plunger; a disc spring, the disc spring opposing actuation of the plunger; a SMA wire that can to drive movement of the plunger within the medication flow path, wherein the positions of the plunger fall within an inclusive range between an actuated and an unactuated position in the medication flow path; and an electrical sensor configured to measure an electrical property of the SMA actuator; and wherein a processor is operably coupled to the electrical sensor and the third SMA wire of the third actuator, the processor configured to execute computer readable instructions to: receive a third electrical property from the electrical sensor of the third actuator; and control the SMA wire of the third actuator, based at least in part on the third electrical property and the one or more signals from the one or more environmental sensors, to drive the movement of the third actuator to positions within the medication flow path correlated with a target medication dispense volume. In some examples, the processor can further execute computer readable instructions to cease heating of the SMA wires of the first actuator or the second actuator, causing the first actuator or second actuator to close.

In some examples, the medication delivery pump includes comprising a base, the base comprising an adhesive at least partially coupled to a tissue site of a patient. In further examples, the delivery pump system comprises a reusable portion, the reusable portion comprising a first housing and the processor, the reusable portion configured to be reversibly coupled to the base. In yet further examples, the delivery pump system comprises a disposable portion, the disposable portion comprising a second housing, the base, and the pump.

In some examples, comprising a medication bladder in fluid communication with the pump. In further examples, some examples, the medication bladder is configured to collapse inward as fluid medication is drawn from the medication bladder by the pump. In some examples, the delivery pump system includes a cannula in fluid communication with the pump. In further examples, the delivery pump system includes a cannula insertion device.

In yet further examples, the cannula insertion device includes: a needle configured to guide the cannula into skin of the user; a spring biasing the needle towards a deployed position; and a trigger component coupled to a third SMA actuator, the trigger component configured to prevent movement of the needle, where, upon actuation of the third SMA actuator, the trigger component releases the needle to allow the spring to move the needle towards the deployed position.

In some examples, the fluid medication comprises insulin or glucagon. In some examples, movement of the plunger and disc spring exerts pressure on the medication flow path. In some examples, the plunger comprises a ridge, the ridge configured to create a seal with an interior cavity of the pump, wherein movement of the position of the seal exerts pressure on the medication flow path. In some examples, the plunger, in the unactuated position, blocks the medication flow path.

In an aspect, the present disclosure provides for a method of dispensing a fluid from a medication pump system comprising a first shape memory alloy (SMA) actuator and a second SMA actuator positioned in series along a flow path, the method including: sensing an environmental condition using one or more environmental sensors, wherein a processor is configured to receive one or more signals from the one or more environmental sensors; determining, using the processor, a first target electrical property value for the first SMA actuator and a second target electrical property value for the second SMA actuator, wherein the first target electrical property value corresponds to a first target displacement of the first SMA actuator and the second target electrical property value corresponds to a second target displacement of the second SMA actuator, wherein the first target electrical property value and the second target electrical property value are determined based at least in part on the received one or more signals from the one or more environmental sensors, and wherein the processor is configured to receive a first electrical property measurement from the first SMA actuator and a second electrical property measurement from the second SMA actuator; heating the first SMA actuator until the first electrical property measurement reaches the first target electrical property value, causing a first plunger of the first SMA actuator to be displaced; and heating the second SMA actuator until the second electrical property measurement reaches the second target electrical property value, causing a second plunger of the second SMA actuator to be displaced.

In some examples, the method further includes ceasing heating of the first SMA actuator, causing the first plunger to return to an unactuated position, and ceasing heating of the second SMA actuator, causing the second plunger return to an unactuated position. In some examples, the causing the first plunger to close comprises allowing a first spring to return the first plunger to the unactuated position, and causing the second plunger to close comprises allowing a second spring to return the second plunger to the unactuated position. In further examples, the first spring includes a first disc spring, and wherein the second spring comprises a second disc spring. In some examples, the heating the first SMA actuator comprises applying a high PWM duty cycle current to the first SMA actuator, and heating the second SMA actuator comprises applying a high PWM duty cycle current to the second SMA actuator.

In some examples, the method further includes maintaining a thermal steady state of the first SMA actuator, causing the first plunger to remain in an actuated position, and maintaining a thermal steady state of the second SMA actuator, causing the second plunger to remain in an actuated position. In further examples, the method includes maintaining a thermal steady state of the first SMA actuator comprises applying a low PWM duty cycle current to the first SMA actuator, and maintaining a thermal steady state of the second SMA actuator comprises applying a low PWM duty cycle current to the second SMA actuator.

In some examples, the first target electrical property value is a first resistance and the second target electrical property value is a second resistance. In some examples, the first target electrical property value is a first voltage and the second target electrical property value is a second voltage. In some examples, the environmental condition comprises one or more of an ambient temperature, an ambient pressure, or an ambient humidity. In some examples, the first SMA actuator comprises a first muscle wire and the second SMA actuator comprises a second muscle wire. In further examples, the first muscle wire is configured to contract when a first electrical current is applied to the first muscle wire and the second muscle wire is configured to contract when a second electrical current is applied to the second muscle wire. In yet further examples, the first muscle wire and the second muscle wire comprise one or more of nitinol or nickel titanium alloy. In some examples, the method includes generating, via the first plunger and the second plunger, a negative pressure to pull fluid from a medication bladder into the flow path. In some examples, the method includes allowing, via the first plunger and the second plunger, a pressurized fluid to be pushed into the flow path.

In an aspect, the present disclosure provides for a medication delivery pump system, the system including: an actuator configured to manipulate a medication volume passing through a medication flow path of the medication delivery pump system, the actuator including: a plunger; a disc spring, the disc spring opposing actuation of the plunger; a shape memory alloy (SMA) wire configured to drive movement of the plunger, wherein a position of the plunger falls within an inclusive range between an actuated and an unactuated position in the medication flow path; an electrical sensor configured to sense an electrical property of the SMA wire; and a processor operably coupled to the electrical sensor and the SMA wire, the processor configured to execute computer readable instructions to: calibrate, based at least in part on a signal from the electrical sensor, the SMA wire to map a target contraction length of the SMA wire with the position of the plunger; and control the calibrated SMA wire to drive the movement of the plunger to a position within the medication flow path correlated with a target medication dispense volume.

In some examples, the delivery pump system comprising a contact sensor configured to determine whether the plunger is in the actuated position or the unactuated position, and wherein the processor is configured to execute computer readable instructions to calibrate, based at least in part on a signal of the contact sensor, the SMA actuator pump to map the target contraction length of the SMA actuator with the position of the plunger in the medication flow path. In some examples, to calibrate the SMA wire, the processor can determine a target contraction length of the SMA wire based on a target medication dispense volume, and wherein the processor is configured to cause the SMA wire to contract to the target contraction length.

In some examples, the delivery pump system includes at least one environmental sensor, wherein the processor is configured to adjust control of the SMA wire depending at least in part on a signal from the at least one environmental sensor. In further examples, the environmental sensor comprises at least one of a temperature sensor, a pressure sensor, or a humidity sensor. In some examples, the processor is configured to control a heating rate or a heating time of the SMA wire. In some examples, the electrical sensor includes an ohmmeter configured to measure a resistance of the SMA wire, wherein the controller is configured to receive a resistance measurement from the ohmmeter and, based at least in part on the resistance measurement, adjust control of the SMA wire.

In some examples, the delivery pump systems includes a fixed electrical contact, and wherein the actuator comprises a mobile electrical contact configured to abut the fixed electrical contact when the actuator is in an actuated position, where the processor is configured to detect that the actuator is in the actuated position based at least in part on the mobile electrical contact touching the fixed electrical contact.

In some examples, the delivery pump system includes a cannula that can administer a medication to a patient, the actuator configured to control dispensation of the medication from the cannula to the patient. In some examples, the medication comprises insulin or glucagon.

In some examples, the processor can cause a current to be applied to the SMA wire to cause the SMA wire to contract, and wherein the processor is configured to cause the current to be stopped from being applied to the SMA wire. In further examples, the processor can cause pulse width modulation (PWM) of the current applied to the SMA wire. In further examples, the processor can adjust application of the PWM to the SMA wire so as to maintain a contracted position of the plunger. In some examples, the processor is configured to cause the SMA wire to contract to a plurality of target different contraction lengths. In some examples, the actuator is a first actuator of a plurality of actuators, the system comprising: the medication flow path, wherein the first actuator is configured to control flow of a fluid along the medication flow path; and a second actuator positioned along the medication flow path and in series with the first actuator, the second actuator configured to control flow of the fluid along the flow path.

In some examples, the disc spring is configured to oppose contraction of the SMA wire. In some examples, movement of the plunger and disc spring exerts pressure on the medication flow path. In some examples, the plunger comprises a ridge, the ridge configured to create a seal with an interior cavity of the pump, wherein movement of the position of the seal exerts pressure on the medication flow path. In some examples, the plunger, in the unactuated position, blocks the medication flow path.

In an aspect, the present disclosure provides for a method of dispensing fluid from a medication pump comprising a shape memory alloy (SMA) actuator, the method including: determining, using a processor configured to receive measurements of an electrical property of the SMA actuator, a target electrical property value of the SMA actuator corresponding to a target contraction of the SMA actuator; and heating the SMA actuator until a measured electrical property value of the SMA actuator reaches the target electrical property value, causing a plunger of the medication pump to open.

In some examples, the method includes ceasing heating the SMA actuator, causing the plunger to close. In some examples, the step of heating comprises applying a high PWM duty cycle current to the SMA actuator. In some examples, the method includes maintaining a thermal steady state of the SMA actuator, causing the plunger to remain in an open position. In further examples, the step of maintaining a thermal steady state comprises applying a low PWM duty cycle current to the SMA actuator.

In some examples, the target electrical property is a resistance. In other examples, the target electrical property is a voltage. In some examples, the method includes sensing an ambient environmental condition using one or more environmental sensors, wherein the processor is configured to receive one or more signals from the one or more environmental sensor, and wherein the step of determining the target electrical property value comprises determining the target electrical property value based at least in part on the sensed ambient environmental condition. In some examples, the ambient environmental condition comprises an ambient temperature, an ambient pressure, or an ambient humidity.

In an aspect, the present disclosure provides for a method of calibrating a shape memory alloy (SMA) actuator of a medication pump, including:
(a) applying a first electrical power to the SMA actuator;
(b) measuring a first dispense volume of fluid produced by the medication pump due to application of the first electrical power;
(c) applying a second electrical power to the SMA actuator;
(d) measuring a second dispense volume of fluid produced by the medication pump due to application of the second electrical power;
(e) determining a calibration algorithm based at least in part on the first and second electrical powers and first and second volumes dispensed;
(f) storing the calibration algorithm on a processor of the medication pump; and
(g) applying a target electrical power to the SMA actuator to cause dispense of a target volume, wherein the processor is configured to determine the target electrical power based at least in part on the calibration algorithm and the target volume.

In some examples, the method includes carrying out steps (a)-(d) at a first environmental condition and repeating steps (a)-(d) at a second environmental condition, wherein step (d) comprises determining the calibration algorithm based at least in part on the first and second electrical powers and first and second volumes at each of the first and second environmental conditions. In some examples, the method includes sensing the first and second environmental conditions using an environmental sensor of the medication pump. In some examples, the first and second environmental conditions are two different ambient temperatures, two different atmospheric pressures, or two different humidities. In some examples, the medication pump comprises a plurality of SMA actuators, wherein the method comprises repeating steps (a)-(f) for each of the plurality of SMA actuators, and wherein step (g) comprises applying a target electrical power to each SMA actuator, wherein the target electrical power for each SMA actuator is determined by the processor based at least in part on the calibration algorithm of each SMA actuator.

### BRIEF DESCRIPTION OF THE DRAWINGS

Embodiments of various inventive features will now be described with reference to the following drawings. The drawings are provided to illustrate example embodiments described herein and are not intended to limit the scope of the disclosure.

FIG. 1A illustrates an example disease management system that may be part of a disease management environment or used as an interleaved device.

FIG. 1B illustrates an example implementation of a disease management system.

FIGS. 1C-1D illustrate perspective views of a disease management device in accordance with aspects of this disclosure.

FIGS. 1E-1J illustrate various views of an implementation of a disease management device in accordance with aspects of this disclosure.

FIGS. 1K-1-1M illustrate various perspective views of the disease management device of FIGS. 1C-1J in accordance with aspects of this disclosure.

FIG. 1N shows an alternative example of a disease management system where several components are enclosed by a single housing.

FIG. 1O shows an exploded view of the example of FIG. 1N.

FIGS. 2A-2D illustrate perspective views of internal hardware components of the disease management device of FIGS. 1C-1M in accordance with aspects of this disclosure.

FIG. 3A illustrates an example implementation of an efficiently emptying medication bladder.

FIG. 3B illustrates a perspective view of a medication bladder in accordance with aspects of this disclosure.

FIG. 3C illustrates an example implementation of a rigid portion of an efficiently emptying medication bladder.

FIGS. 4A-4B-2 illustrate perspective views of a pump system of the disease management device in accordance with aspects of this disclosure.

FIGS. 4C-4H illustrate various views of an implementation of the pump system in accordance with aspects of this disclosure.

FIG. 4I illustrates a cross-sectional view of the pump system in accordance with aspects of this disclosure.

FIGS. 5A-5B illustrates a perspective view of a plunger assembly of the disease management device in accordance with aspects of this disclosure.

FIG. 5C illustrates a perspective view of a plunger of the plunger assembly in accordance with aspects of this disclosure.

FIGS. 5D and 5E illustrate exploded perspective views of plungers of the plunger assembly in accordance with aspects of this disclosure.

FIGS. 5F-5K illustrate various views of an implementation of a plunger in accordance with aspects of this disclosure.

FIG. 5L illustrates a cross-sectional view of a plunger in accordance with aspects of this disclosure.

FIGS. 6A and 6B illustrate example components of another example muscle wire pump of a disease management system.

FIGS. 7A-7C illustrate views of example plunger and silicone membrane, which acts as a spring component of an example muscle wire pump of a disease management system.

FIGS. 8A-8C illustrate views of an example connection of plunger and silicone membrane, which acts as a spring component to an example fluid line of an example muscle wire pump.

FIGS. 9A-9C-2 illustrate an example functioning of a spring component of an example muscle wire pump.

FIGS. 10A-10B illustrates example operation of an example muscle wire pump.

FIG. 11 illustrates an example flow path of a muscle wire pump.

FIGS. 12A-12C illustrates example components of a feedback control system that may be part of a muscle wire pump.

FIG. 13A illustrates an example muscle wire pump that includes a feedback control system.

FIG. 13B illustrates an exploded view of the muscle wire pump of FIG. 13A.

FIG. 14 illustrates a cross-sectional view of an example muscle wire pump.

FIGS. 15A-15C schematically illustrate an example pump including a shape memory alloy (SMA) actuator and volume dispense control features.

FIGS. 15D-15F schematically illustrate a second example pump including a SMA actuator and volume dispense control features.

FIGS. 15G-15I schematically illustrate a third example pump including a SMA actuator and volume dispense control features.

FIG. 16 illustrates an example method of controlling an example pump of FIGS. 15A-15C.

FIG. 17 is an example actuator calibration curve for the pump of FIGS. 15A-15C.

FIG. 18 are example actuator calibration curves for two different ambient environmental conditions for the pump of FIGS. 15A-15C.

FIG. 19 are example actuator calibration curves for two different pumps in accordance with FIGS. 15A-15C.

FIG. 20 illustrates an example routine that may be performed by a disease management system.

FIG. 21 illustrates an example process of releasing medication from an example of a medication bladder.

FIGS. 22A-22B illustrate perspective views of an injection system of the disease management device in accordance with aspects of this disclosure.

FIGS. 22C-22H illustrate various views of an implementation of the injection system in accordance with aspects of this disclosure.

FIG. 22I illustrates an exploded perspective view of the injection system of the disease management device in accordance with aspects of this disclosure.

FIGS. 22J-22K illustrate perspective views of a trigger release of the injection system in accordance with aspects of this disclosure.

FIG. 22L illustrate a view of the injection system in accordance with aspects of this disclosure.

FIGS. 22M-22N illustrate a view of a cannula assembly and needle assembly of the injection system in accordance with aspects of this disclosure.

FIGS. 22O-22T illustrate various views of an implementation of the needle assembly in accordance with aspects of this disclosure.

FIG. 23A illustrates a cross-section side view of an automated needle insertion and needle removal device prior to launch.

FIG. 23B illustrates a cross-section side view of an automated needle insertion and needle removal device with the muscle wire actuated.

FIG. 23C illustrates a cross-section side view of an automated needle insertion and needle removal device with a needle in an inserted position.

FIG. 23D illustrates a cross-section side view of an automated needle insertion and needle removal device with a needle in a retracted position.

FIG. 23E illustrates an example process for an automated needle insertion and needle removal device.

FIG. 24A illustrates a perspective view of a first cover and a second cover of the disease management device in accordance with aspects of this disclosure.

FIGS. 24B-24G illustrate various views of an implementation of the first cover in accordance with aspects of this disclosure.

FIGS. 24H-24M illustrate various views of an implementation of the second cover in accordance with aspects of this disclosure.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims that may arise here from is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Medications for management of diabetes or other physiological condition, can be administered by a minimally-invasive wearable device, such as a medication delivery pump or the like. It may be desirable that such systems are consistent and accurate. Over-delivering or under-delivering medication may lead to adverse health consequences. With respect to delivery of insulin, for example, delivering too much insulin to a patient may result in hypoglycemia, which can lead to loss of consciousness, comas, or even death. Delivering too little insulin may lead to hyperglycemia which may cause significant damage to internal organs such as the heart.

As discussed herein, infusion pumps using shape memory alloy (SMA) actuators can be precisely controlled. Deformation (e.g., retraction) of the SMA actuator can be thermally induced, for example by driving an electrical current through the SMA actuator. An electrical resistance of the SMA actuator can be measured to determine displacement of the plunger. Changes in environmental conditions may be accounted for when controlling such SMA actuators to ensure tight conformity to standard performance. In some examples, an environmental sensor can sense an environmental condition and the control of the SMA actuator can be adjusted in response to the sensed environmental condition.

### Overview of Disease Management System With Internal Hardware

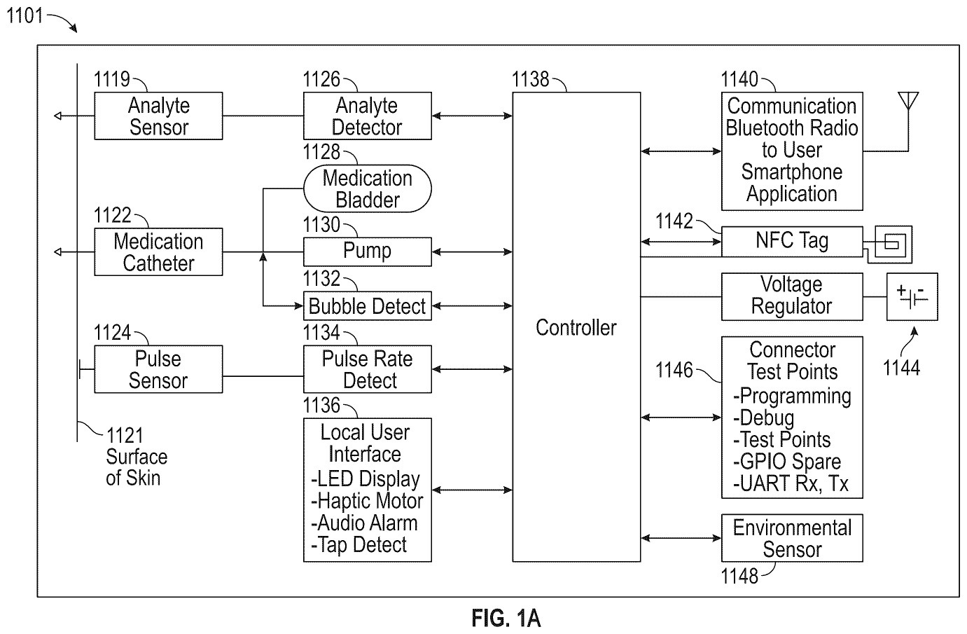

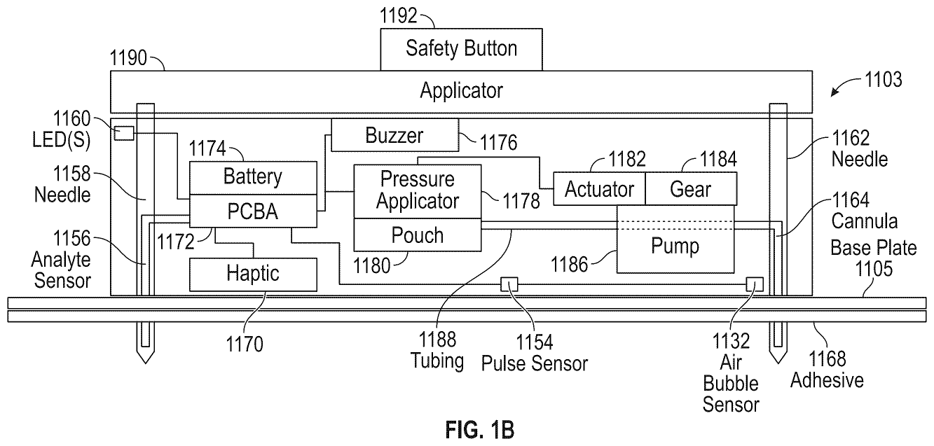

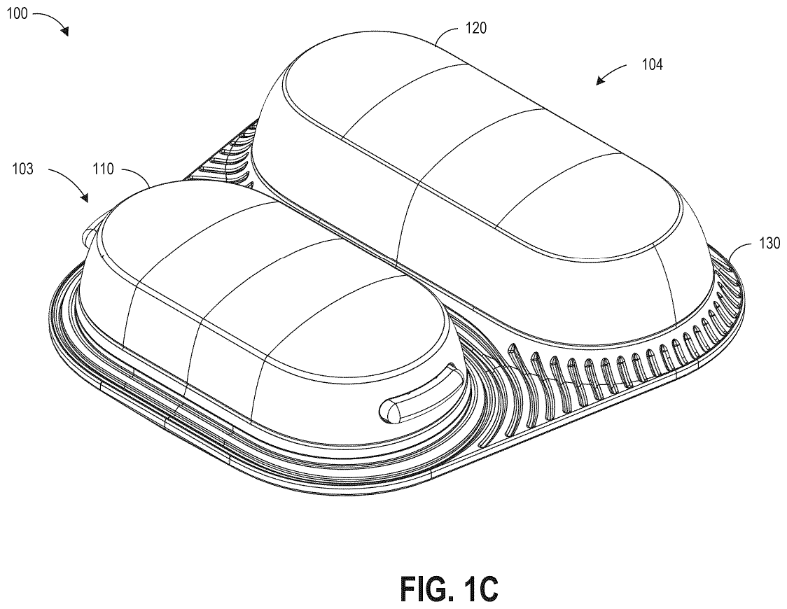

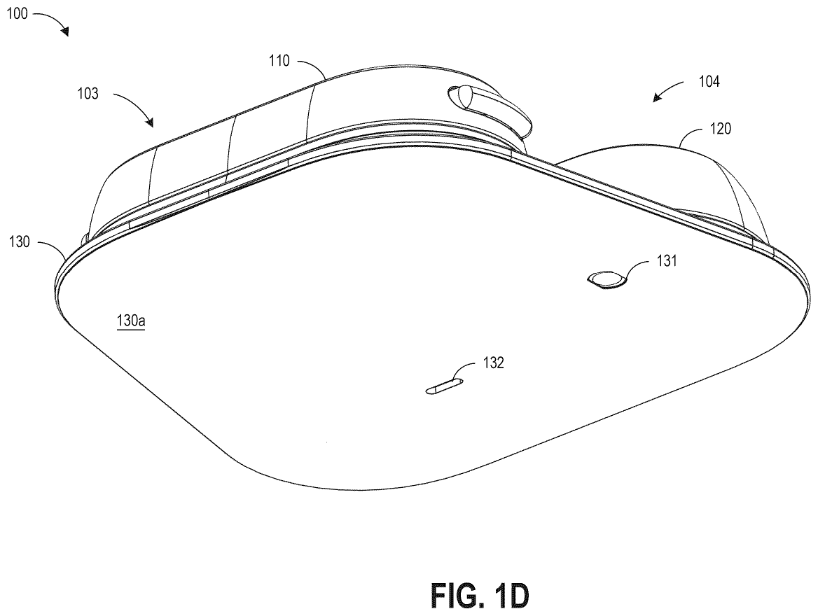

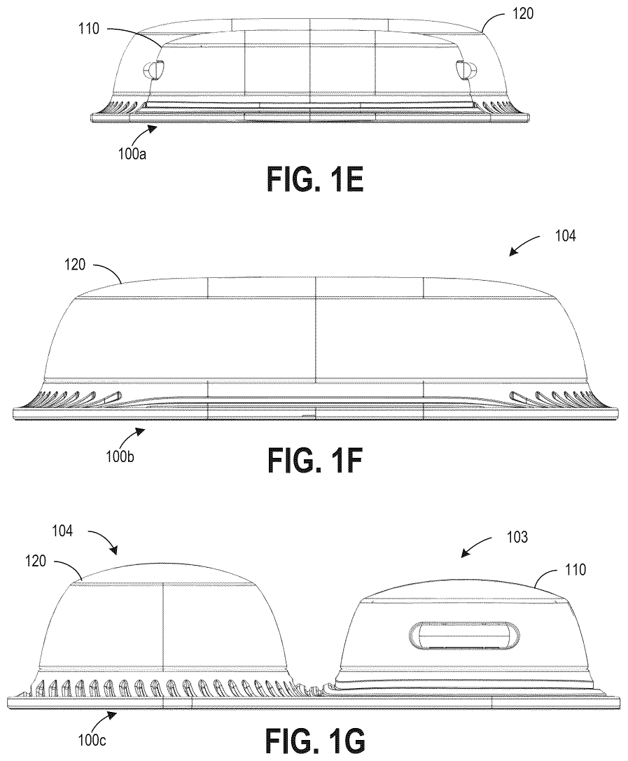

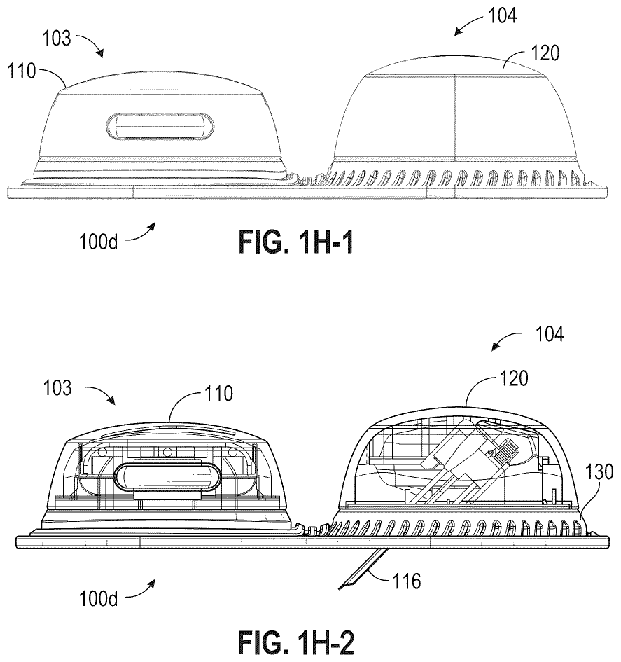

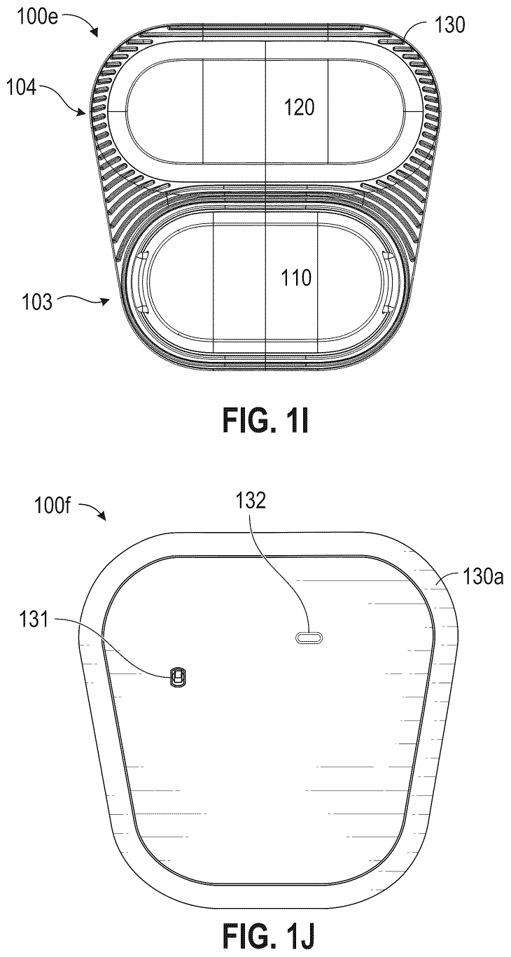

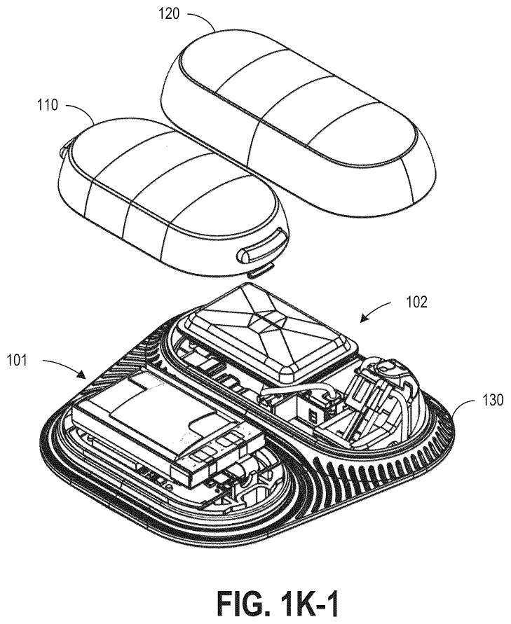

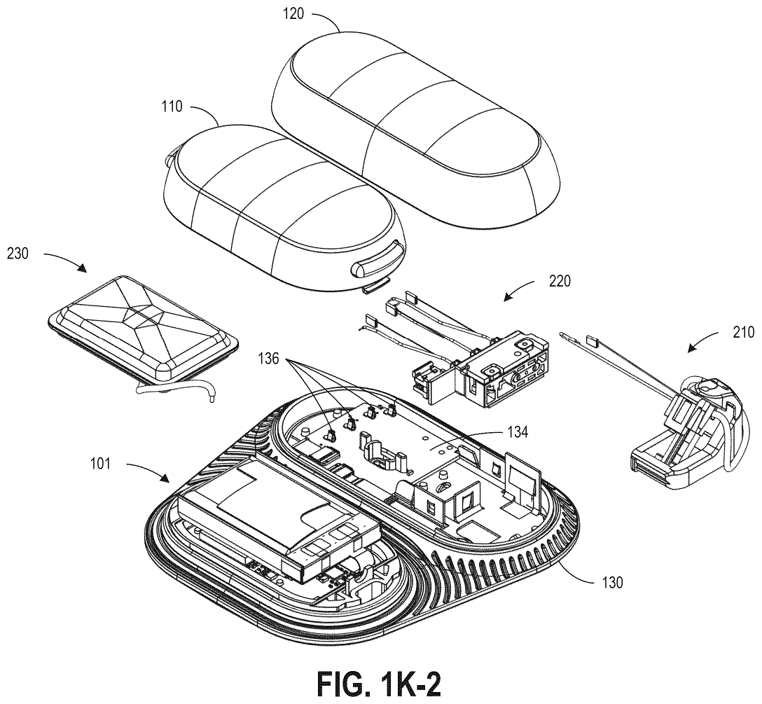

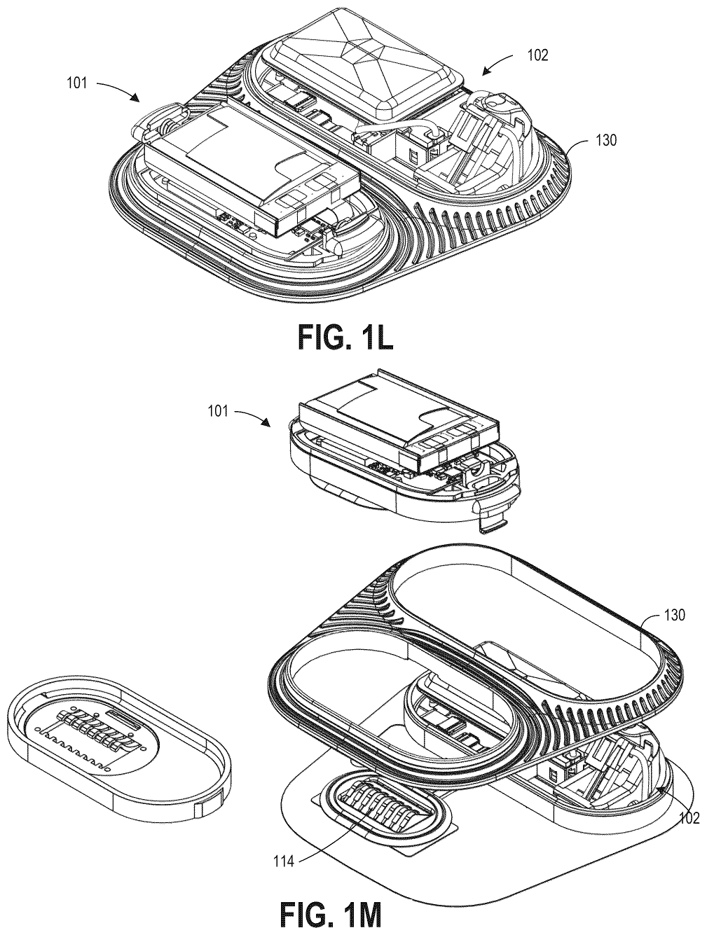

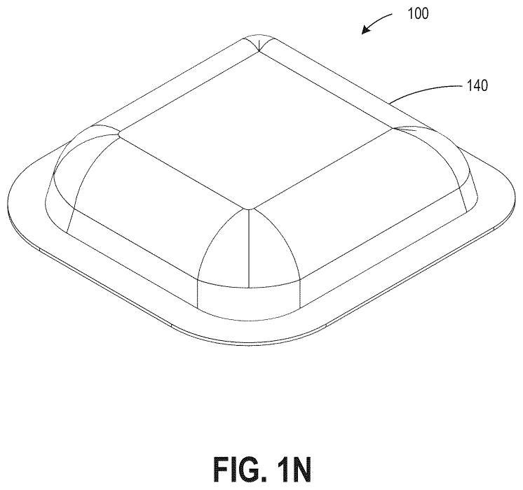

FIG. 1A shows a block diagram of an example disease management system 1101. In some examples, the disease management system 1101 may be part of a disease management environment including more than one device. A disease management system 1101 may be configured to measure one or more physiological parameters of a patient (such as pulse, skin temperature, or other values), measure one or more analytes present in the blood of a patient (such as glucose, lipids, or other analyte) and administer medication (such as insulin, glucagon, or other medication). In some examples, a disease management system 1101 may be configured to communicate with one or more hardware processors that may be external to the disease management system 1101, such as a cloud-based processor or user device. A disease management system 1101 may include an NFC tag to support authentication and pairing with a user device (for example, smart phone or smart watch), Bluetooth communication with additional disease management systems or devices, and Bluetooth communication with a paired user device running an associated control application. To support ease of use and safe interaction with the patient, the system may incorporate user input through a tap-detecting accelerometer and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

As illustrated in FIG. 1A, a controller 1138 of the disease management system 1101 may be configured to communicate and control one or more components of the disease management system 1101. The controller 1138 may include one or more hardware processors, such as a printed circuit board (PCB) or the like. The controller 1138 may be configured to communicate with peripheral devices or components to support the accurate measurement of physiological parameters and blood analytes, such as patient pulse, patient temperature, and blood glucose, using detector electronics. The controller 1138 may subsequently calculate dose or receive a calculated dose value and administer medication, such as insulin, by actuation of an actuated pump. The controller 1138 may record device activity and transfer the recorded data to non-volatile secure memory space. At the end of the life of a device or system, the controller can be configured to lock operation, and create a data recovery module to permit authenticated access to the recorded data if needed.

A disease management system 1101 may include an analyte sensor 1119. The analyte sensor 1119 may be configured to detect analytes in the patient's blood. For example, an analyte sensor 1119 can include a glucose sensing probe configured to pierce the surface of the skin 1121. In some examples, a disease management system 1101 may include a plurality of analyte sensors 1119 to detect one or more analytes. In some examples, an analyte sensor 1119 may be configured to detect a plurality of analytes. Sensed analytes may include, but are not limited to, glucose, insulin, and other analytes. An analyte sensor 1119 may be configured to communicate with an analyte detector 1126. The analyte detector 1126 may be configured to receive a signal of one or more analyte sensors 1119 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

A disease management system 1101 may include a medication catheter 1122. The medication catheter 1122 may be configured to administer medication, including, but not limited to insulin, to the patient. The medication catheter 1122 may receive medication from a medication bladder 1128 configured to contain medication to be administered. The medication bladder 1128 may be configured to contain medication for a prolonged period, such as 1 day, 3 days, 6 days, or more. The medication bladder 1128 may be configured to contain certain medication types, such as insulin. In some examples, a disease management system 1101 may include a plurality of medication bladders 1128 for one or more reservoirs of the same or different medications. In some examples, a disease management system 1101 may be configured to mix medications from medication bladders 1128 prior to administration to the patient. A pump 1130 may be configured to cause medication to be administered from the bladder 1128 to the patient through the medication catheter 1122. A pump 1130 may include, but is not limited to, a pump such as described herein.

A disease management system 1101 may optionally include a physiological sensor 1124. The physiological sensor 1124 may include a pulse rate sensor, patient temperature sensor, pulse oximeter, the like or a combination thereof. In some examples, a disease management system 1101 may be configured to include a plurality of physiological sensors. The physiological sensor 1124 may be configured to communicate with a physiological detector 1134. The physiological detector 1134 may be configured to receive a signals of the physiological sensor 1124. The physiological detector 1134 may be configured to measure or determine and communicate a physiological value from the signal. The physiological detector 1134 may be configured to communicate with the controller 1138. For example, the physiological detector 1134 may be configured to, for example, send measured physiological values to the controller 1138 and receive control signals from the controller.

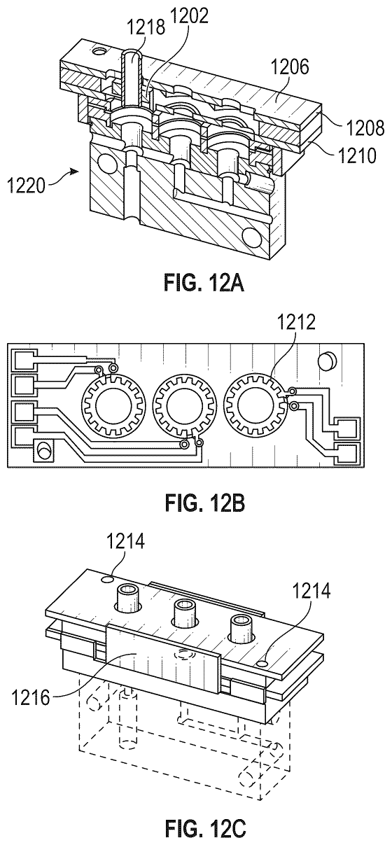

The disease management system 1101 may optionally include one or more environmental sensor(s) 1148. The one or more environmental sensor(s) 1148 may be capable of detecting ambient environmental conditions of the disease management system 1101, for example an ambient temperature, an ambient pressure, and/or an ambient humidity, among other conditions. The environmental sensor(s) 1148 may be positioned within the housing including the pump 1130. The environmental sensor(s) 1148 may be positioned away from skin 1121 to allow for more accurate measurement of environmental conditions (e.g., environmental temperature) rather than, for instance, conditions of the user (e.g., body temperature). The controller 1138 may be in communication with the one or more environmental sensor(s) 1148. The controller 1138 may adjust control of the pump 1130 based on the environmental conditions detected by the one or more environmental sensor(s) 1148, especially in examples where the pump 1130 includes shape memory alloy (SMA) actuators. Control of such pumps is provided for herein, for example with reference to FIG. 12A-19.

A disease management system 1101 may include one or more local user interfacing components 1136. For example, a local user interfacing component 1136 may include, but is not limited to one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, the like or a combination thereof. In some examples, a user input detector may include an inertial measurement unit, tap detector, touch display, or other component configured to accept and receive user input. In some examples, audio speakers may be configured to communicate audible alarms related to device status, medication status user status, the like or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include one or more communication components 1140. A communication component 1140 can include but is not limited to one or more radios configured to emit Bluetooth, cellular, Wi-Fi, or other wireless signals. In some examples, a communication component 1140 can include a port for a wired connection. Additionally, a disease management system 1101 may include an NFC tag 1142 to facilitate in communicating with one or more hardware processors. The one or more communication components 1140 and NFC tag 1142 may be configured to communicate with the controller 1138 in order to send and/or receive information associated with the disease management system 1101. For example, a controller 1138 may communicate medication information and measured values through the one or more communication components 1140 to an external device. Additionally, the controller 1138 may receive instructions associated with measurement sampling rates, medication delivery, or other information associated with operation of the management system 1101 through the one or more communication components 1140 from one or more external devices.

A disease management system 1101 may include one or more power components 1144. The power components may include but are not limited to one or more batteries and power management components, such as a voltage regulator. Power from the one or more power components 1144 may be accessed by the controller and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake-up mode. The wake-up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculation, are performed and data is communicated to an external device and/or the cloud. In another example, a disease management system 1101 may include a pump mode where the system is configured to enter a higher power mode where the pump draws power to deliver medication to the patient.

A disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, the like or a combination thereof.

Various components shown in FIG. 1A can be optionally included in the disease management system 1101, including the physiological detector 1134, the physiological sensor 1124, the analyte sensor 1119, the analyte detector 1126, the air bubble sensor 1132, the components 1136, the one or more communication components 1140, the NFC tag 1142, the one or more power components 1144, the one or more connector test points 1146, and/or the environmental sensor 1148.

FIG. 1B illustrates an example implementation of a disease management system 1103 and applicator 1190 for applying a disease management system 1103 to a patient. The disease management system 1103 can include any one or more of the features discussed above with respect to the disease management system 1101 in addition to the features described below. In the illustrated example, an applicator 1190 may be configured to mate with the disease management system 1103. In some examples, an applicator 1190 may include a safety button 1192 for release or other interaction with the applicator 1190. In the illustrated example, a disease management system 1103 may include one or more LEDs 1160 that may be configured to output information using one or more of color, frequency, and length of display. In some examples, the disease management system 1103 may include a buzzer 1176, haptic actuator 1170, or other feedback mechanism, such as a speaker to output information to the patient, such as an alarm. In some examples, a disease management system 1103 may include a battery 1174, and/or a controller 1172. In some examples, a disease management system 1103 may include aspects of a medication administration system, such as a bladder 1180 (also referred to as a pouch herein), a bladder pressure applicator 1178 to provide pressure on the bladder 1180 (such as a component of a pump), actuator 1182, pump gears 1184, and a pump 1186. The pump 1186 may be in fluid communication with the bladder 1180 and/or the one or more cannulas 1164 via tubing 1188. In some examples, a disease management system 1103 may include one or more needles 1158 that may include one or more analyte sensors (such as a glucose sensor) 1156. In other examples, the bladder 1180 can be capable of collapsing on its own (i.e., without a bladder pressure applicator 1178) in response to liquid medication departing the bladder 1180. In some examples, a disease management system 1103 may include one or more needles 1162 that may include one or more cannulas 1164 configured to administer medication to the patient. In some examples, a disease management system 1103 may include an air bubble sensor 1132 configured to detect the presence of air bubbles in the medication prior to delivery to the patient. In some examples, a disease management system 1103 may include one or more physiological sensors 1154, such as a non-invasive physiological sensor including but not limited to a pulse sensor. In some examples, the disease management system 1103 may include a base plate 1105 and an adhesive layer 1168 below the base plate 1105 to provide adhesion of the disease management system 1103 to the patient's skin. As described below, a housing of the disease management system 1103 may consist of a combination of flexible and rigid material so as to both provide support for the components of the disease management system 1103 and allow conforming, at least in part, of the disease management system 1103 to the skin of the patient.

The adhesive layer 1168 may be configured to provide adhesion for a prolonged period. For example, the adhesive layer 1168 may be configured to adhere the disease management system 1103 to the skin of a patient for a period of 1 day, 3 days, 6 days, or more or fewer days or hours. In some examples, the adhesive layer 1168 may be configured to have an adhesive force sufficient to prevent accidental removal or movement of the disease management system 1103 during the intended period of use of the disease management system 1103. In some examples, the adhesive layer 1168 may be a single layer of adhesive across at least a portion of a surface the disease management system 1103 that is configured to interface with the patient. In some examples, the adhesive layer 1168 may include a plurality of adhesive areas on a surface of the disease management system 1103 that is configured to interface with the patient. In some examples, the adhesive layer 1168 may be configured to be breathable, adhere to the patient's skin after wetting by humidity or liquids such as tap water, saltwater, and chlorinated water. A thickness of the adhesive may be, for example, in a range of 0.1 to 0.5 mm or in a range of more or less thickness.

In some examples, a needle 1158, 1162 may be inserted at different depths based on a patient age, weight, or other parameter. For example, a depth of insertion of a medication cannula may be approximately 3 mm for 7 to 12 year olds. In another example, a depth of insertion of a medication cannula may be approximately 4 mm for 13 year olds and older. In another example, a depth of insertion of a medication needle may be approximately 4 to 4.5 mm for 7 to 12 year olds. In another example, a depth of insertion of a medication needle may be approximately 5 to 5.5 mm for 13 year olds and older. In another example, a depth of insertion of an analyte sensor may be approximately 3 mm for 7 to 12 year olds. In another example, a depth of insertion of an analyte sensor may be approximately 4 mm for 13 year olds and older. In another example, a depth of insertion for a needle associated with an analyte sensor may be approximately 4 to 4.5 mm for 7 to 12 year olds. In another example, a depth of insertion for a needle associated with an analyte sensor may be approximately 5 to 5.5 mm for 13 year olds and older. However, other values or ranges for any of the inserted components are also possible.

As described above, closed loop medication administration systems, such as closed loop insulin administration systems, can improve the quality of life of a patient who requires regular administration of medication and monitoring of various physiological and/or other parameters. The patient's quality of life can be further improved as more components of and/or supporting a closed loop medication administration system are incorporated into a disease management system.

In various implementations, a disease management system can include some or all components of a closed loop medication administration system in a self-contained unit. The disease management system may be applied on or worn by a patient allowing for ease of installation and removal of the disease management device. However, other applications may also be possible.

Now with reference to an illustrative example, the figures herein show an example disease management device 100 (or components thereof), which may be part of a disease management system, such as the closed-loop diabetes management environment described herein. A disease management device may measure one or more physiological parameters of a patient (such as pulse, skin temperature, or other values), measure one or more analytes present in the blood of a patient (such as glucose, lipids, or other analytes) and administer medication (such as insulin, glucagon, or other medication). The device 100 may incorporate some or all of the features discussed with reference to FIGS. 1A and 1B above.

FIG. 1C illustrates a top perspective view of the device 100. The device 100 includes first portion 103 including, among other things, a first cover 110, and a second portion 104, including, among other things, a second cover 120, and a base 130. FIG. 1D illustrates a bottom perspective view of the device 100. As illustrated, a bottom side 130a of the base 130 includes a first opening 131, and a second opening 132. The base 130 may couple to a tissue site of a patient, for example, with the bottom side 130a coupling to the tissue site. The base 130 may couple to a user via an adhesive of the bottom side 130a. In this way, the first opening 131 may provide for an injection system (such as disclosed herein) to pass a needle through the first opening 131 to the tissue site. In this way, the injection system may contact a user.

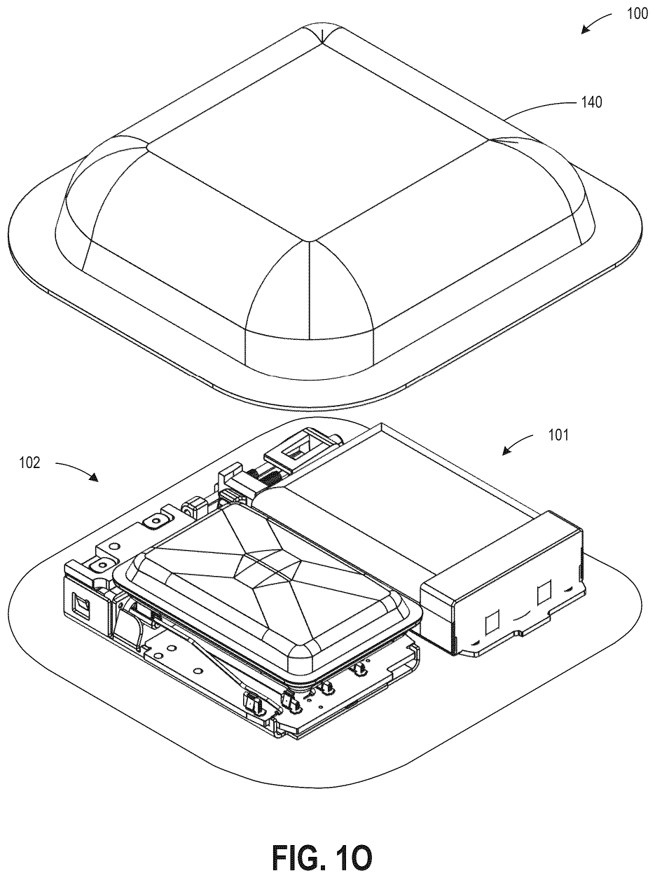

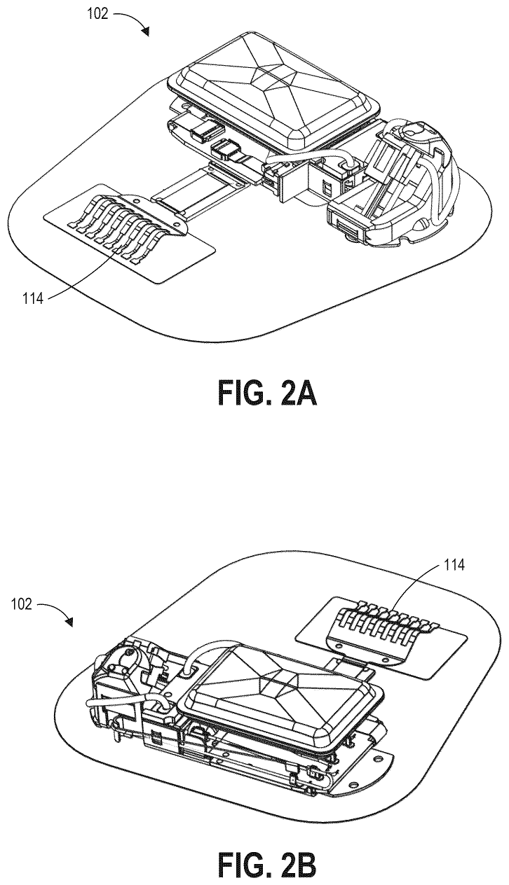

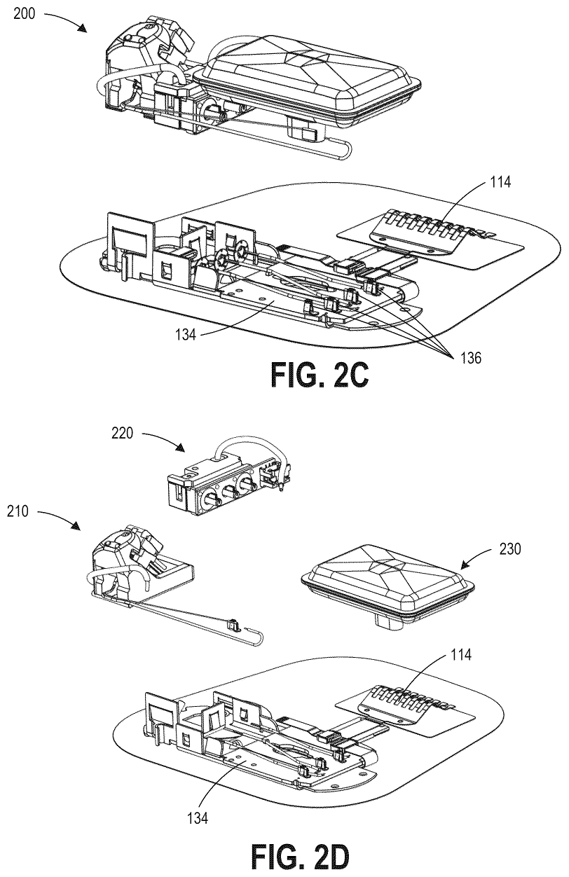

FIGS. 1E-1H-2 illustrate various views of the device. FIG. 1E shows the device 100 from a first view 100a. FIG. 1F shows the device 100 from a second view 100b. FIG. 1G shows the device 100 from a third view 100c. FIG. 1H-1 shows the device 100 from a fourth view 100d. FIG. 1H-2 shows the device 100 from the same fourth view as FIG. 1H-1, with portions of the device made transparent and the needle 116 deployed. FIGS. 1I-1J show the device 100 from a top view 100e and a bottom view 100f, respectively.

FIGS. 1K-1-1L show the device 100 with covers 110, 120 separated from the device 100. As illustrated in FIGS. 1K-1-1L, the device 100 may include first device hardware 101 of the first portion 103 and second device hardware 102 of the second portion 104. FIG. 1K-2 illustrates an exploded view of the device 100 showing the injection system 210, the pump system 220, the medication bladder 230, and the first device hardware 101. Also shown is a PCB 134 and a plurality of anchors 136. Within the device 100, the medication bladder 230 and pump system 220 can at least partially be positioned over and/or coupled to the PCB 134. The plurality of anchors 136 can be attached to the PCB 134. The plurality of anchors 136 can couple to ends of SMA actuators of the pump system 220 and/or the injection system 210 to give a stationary component against which the SMA actuators can pull. FIG. 1M illustrates an exploded perspective view of the device 100, with the first device hardware 101 separated from the second device hardware 102, and the second device hardware 102 separated from the base 130. In some examples, the first device hardware 101 may couple to the second device hardware 102. The first device hardware 101 may include one or more hardware processors. The one or more processors can be in electrical communication with the electrodes 114 when the first portion 103 is coupled to the base 130. The electrodes 114 may allow the one or more hardware processors of the first device hardware 101 to communicate with the second device hardware 102. Various components of the second device hardware 102 will be discussed with reference to FIGS. 2A-5L.

FIG. 1N shows an alternative example of a device 100 where components discussed with reference to the first device hardware 101 and the second device hardware 102 above are enclosed by a single housing 140. FIG. 1O shows an exploded view of the device 100. In some examples, the device 100 can include both the first device hardware 101 and the second device hardware 102 within a single housing 140. In some such examples, the entire device 100 may be disposable. In other examples, portions of the device 100 may be disposable while other portions of the device 100 may be reusable. In some examples, the single housing 140 can be removable to allow for replacement or disposal of portions of the second device hardware 102. In some such examples, the first device hardware 101 may be reusable.

In some examples, the device 100 (and components thereof) may be similar or identical to and/or incorporate any of the features described and/or illustrated with respect to any of the devices, assemblies, systems, and/or methods described and/or illustrated in U.S. application No. Ser. No. 17/161,528, filed Jan. 28, 2021 and titled “METHOD OF OPERATING REDUNDANT STAGGERED DISEASE MANAGEMENT SYSTEMS,” the disclosure of which is hereby incorporated by reference in its entirety, and which claims priority to U.S. Provisional Application No. 62/968,107, which was filed on Jan. 30, 2020 and is titled “CLOSED LOOP INSULIN DELIVERY SYSTEM,” and also claims priority to U.S. Provisional Application No. 62/978,480, which was filed on Feb. 19, 2020 and is titled “REDUNDANT STAGGERED GLUCOSE SENSOR INSULIN DOSAGE SYSTEM,” and also claims priority to U.S. Provisional Application No. 63/015,272, which was filed on Apr. 24, 2020 and is titled “REDUNDANT STAGGERED GLUCOSE SENSOR INSULIN DOSAGE SYSTEM,” and also claims priority to U.S. Provisional Application No. 63/044,831, which was filed on Jun. 26, 2020 and is titled “REDUNDANT STAGGERED GLUCOSE SENSOR INSULIN DOSAGE SYSTEM,” the disclosures of which are expressly incorporated by reference herein in its entirety for all purposes. Further, the device 100 may be similar or identical to and/or incorporate any of the features described and/or illustrated with respect to any of the devices, assemblies, systems, and/or methods described and/or illustrated in PCT Application No. PCT/US2021/015559, which was filed on Jan. 28, 2021 and is titled “REDUNDANT STAGGERED GLUCOSE SENSOR DISEASE MANAGEMENT SYSTEM,”the disclosure of which is hereby incorporated by reference in its entirety.

In some examples, the device 100 may be similar or identical to and/or incorporate any of the features described and/or illustrated with respect to any of the devices, assemblies, systems, and/or methods described and/or illustrated in U.S. application No. Ser. No. 18/155,978, filed Jan. 18, 2023 and titled “MODULAR WEARABLE DEVICE FOR PATIENT MONITORING AND DRUG ADMINISTRATION,” the disclosure of which is hereby incorporated by reference in its entirety, and which claims priority to U.S. Provisional Application No. 63/300,426, which was filed on Jan. 18, 2022 and is titled “MODULAR WEARABLE DEVICE FOR PATIENT MONITORING AND DRUG ADMINISTRATION,” the disclosure of which is expressly incorporated by reference herein in its entirety for all purposes.

In some examples, the device 100 may be similar or identical to and/or incorporate any of the features described and/or illustrated with respect to any of the devices, assemblies, systems, and/or methods described and/or illustrated in U.S. application No. Ser. No. 17/817,613, filed Aug. 4, 2022 and titled “MEDICATION DELIVERY PUMP FOR REDUNDANT STAGGERED GLUCOSE SENSOR INSULIN DOSAGE SYSTEM,” the disclosure of which is hereby incorporated by reference in its entirety, and which claims priority to U.S. Provisional Application No. 63/229,305, which was filed on Aug. 4, 2021 and is titled “MEDICATION DELIVERY PUMP FOR REDUNDANT STAGGERED GLUCOSE SENSOR INSULIN DOSAGE SYSTEM,” the disclosure of which is expressly incorporated by reference herein in its entirety for all purposes. Further, the device 100 may be similar or identical to and/or incorporate any of the features described and/or illustrated with respect to any of the devices, assemblies, systems, and/or methods described and/or illustrated in PCT Application No. PCT/US2022/039477, which was filed on Aug. 4, 2022 and is titled “MEDICATION DELIVERY PUMP HAVING GLUCOSE SENSOR,”the disclosure of which is hereby incorporated by reference in its entirety.

In some examples, the device 100 may be similar or identical to and/or incorporate any of the features described and/or illustrated with respect to any of the devices, assemblies, systems, and/or methods described and/or illustrated in U.S. application No. Ser. No. 18/605,630, filed Mar. 14, 2024 and titled “MODULAR DISEASE MANAGEMENT DEVICE AND AUTOMATED NEEDLE AND CANNULA INSERTION DEVICE,” the disclosure of which is hereby incorporated by reference in its entirety, and which claims priority to U.S. Provisional Application No. 63/490,601, which was filed on Mar. 16, 2023 and is titled “MODULAR DISEASE MANAGEMENT DEVICE AND AUTOMATED NEEDLE AND CANNULA INSERTION DEVICE,” the disclosure of which is expressly incorporated by reference herein in its entirety for all purposes. Further, the device 100 may be similar or identical to and/or incorporate any of the features described and/or illustrated with respect to any of the devices, assemblies, systems, and/or methods described and/or illustrated in PCT Application No. PCT/US2024/020096, which was filed on Mar. 15, 2024 and is titled “MODULAR DISEASE MANAGEMENT DEVICE AND AUTOMATED NEEDLE AND CANNULA INSERTION DEVICE,” the disclosure of which is hereby incorporated by reference in its entirety.

In some examples, the device 100 may be similar or identical to and/or incorporate any of the features described and/or illustrated with respect to any of the devices, assemblies, systems, and/or methods described and/or illustrated in U.S. application No. Ser. No. 18/419,408, filed Jan. 22, 2024 and titled “MEDICATION BLADDER FOR MEDICATION STORAGE,” the disclosure of which is hereby incorporated by reference in its entirety, and which claims priority to U.S. Provisional Application No. 63/481,365, which was filed on Jan. 24, 2023 and is titled “CONTAINER FOR MEDICATION STORAGE,” the disclosure of which is expressly incorporated by reference herein in its entirety for all purposes. Further, the device 100 may be similar or identical to and/or incorporate any of the features described and/or illustrated with respect to any of the devices, assemblies, systems, and/or methods described and/or illustrated in PCT Application No. PCT/US2024/012461, which was filed on Jan. 22, 2024 and is titled “MEDICATION BLADDER FOR MEDICATION STORAGE,” the disclosure of which is hereby incorporated by reference in its entirety.

In some examples, the device 100 may be similar or identical to and/or incorporate any of the features described and/or illustrated with respect to any of the devices, assemblies, systems, and/or methods described and/or illustrated in U.S. Provisional Application No. 63/682,899, filed Aug. 14, 2024 and titled “MICROINFUSION PUMP FOR DISPENSING PRECISE VOLUMES,” the disclosure of which is hereby incorporated by reference in its entirety.

FIGS. 2A-2B show various perspective views of the second device hardware 102. As illustrated in FIG. 2A, a top-front perspective view of the second device hardware 102. FIG. 2B illustrates a top-back perspective view of the second device hardware 102. FIG. 2C shows internal components 200 separated from the second device hardware 102. FIG. 2D shows an exploded view of the internal components 200. The internal components 200 including an injection system 210, a pump system 220, and a medication bladder 230.

### Medication Bladder

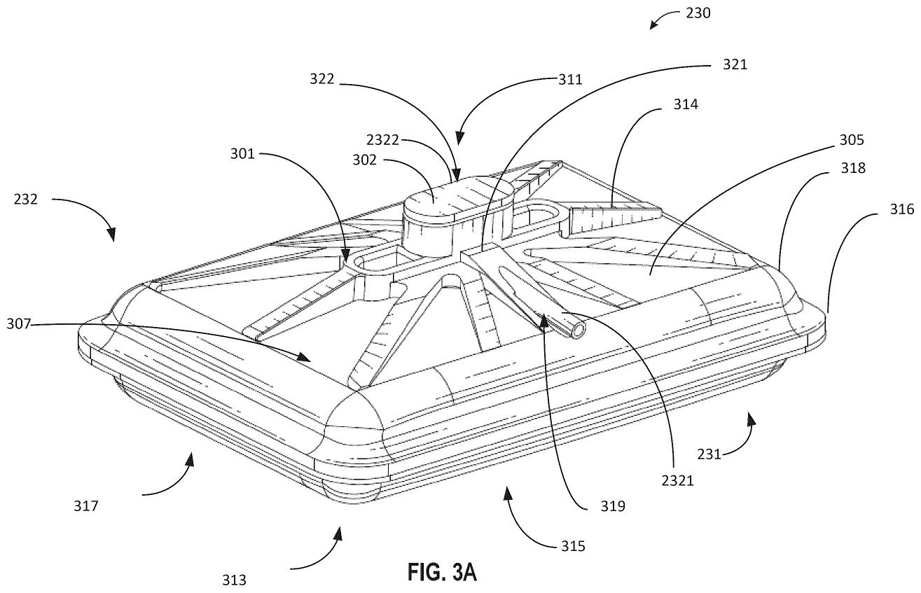

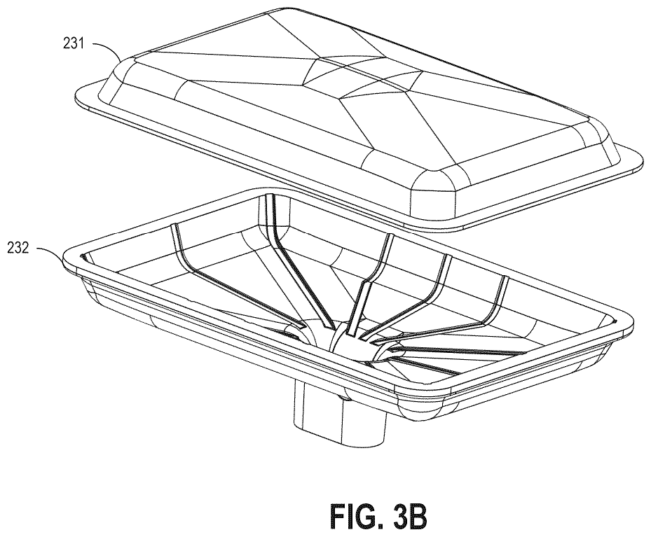

The medication bladder 230 can store fluid medication within the device 100 for pumping by the pump system 220. With reference to an illustrative example, FIG. 3A illustrates a perspective bottom view of the medication bladder 230. The medication bladder 230 may include a first portion 231 and a second portion 232. Aspects of the present disclosure provide the medication bladder 230 that can release fluid including chemical substances, such as medication for management of diabetes or other physiological condition. The medication bladder 230 may reliably release the fluid at different levels of fullness of the bladder and reduce medication waste by facilitating improved emptying of the medication bladder. For example, the medication bladder 230 may include a flexible portion (e.g., a first portion 231) and a rigid portion (e.g., a second portion 232). The second portion 232 may include at least an outlet port 2321 and a plurality of channels 2324 (as disclosed herein, for example, in FIG. 3C) which may facilitate release of the medication in response to an applied force (for example, from a pump as disclosed herein). In some examples, the flexible portion may apply force through application of pressure against the medication in the medication bladder. This may facilitate the flow of fluid through an outlet (such as an output port 2321) of the medication bladder 230. In some examples, the flexible portion may collapse (e.g., move inward) to the rigid portion to facilitate a fuller release of medication from the medication bladder 230. The medication bladder 230 may have the cavity 2323, for example, through which to allow fluid (such as medication) to pass.

FIG. 3B shows a first portion 231 of the medication bladder 230 separating from a second portion 232 of the medication bladder 230. For example, the medication bladder 230 may be similar or identical to and/or incorporate any of the features described and/or illustrated with respect to any of the devices, assemblies, systems, and/or methods described and/or illustrated in U.S. Pub. No. 2024/0245855, the disclosure of which is hereby incorporated by reference in its entirety.

The example medication bladder 230 that may be part of a disease management system, such as disease management system 1101 of FIG. 1A and disease management system 1103 of FIG. 1B. As previously discussed, the second portion 232 can be rigid while the first portion 231 may be flexible and/or pliable. FIG. 3C illustrates a view of an interior side of a second portion 232 showing the interior surface 405 of the second portion 232. As illustrated, the interior surface 405 may be on an opposing side of an exterior surface 305. For example, the interior surface 405 may be on an interior side of the second portion 232, such as within an enclosure formed by second portion 232 and first portion 231, where the interior surface 405 forms at least part of the interior bounds of the enclosure. The second portion 232 can include a plurality of channels 2324. The plurality of channels 2324 may further facilitate the flow of fluid by directing the fluid towards an output (such as the output port 2321).

In some examples, the medication bladder 230 may include the features described above with respect to medication bladder 1128 of FIG. 1A and/or medication bladder 1180 of FIG. 1B in addition to features described below. However, other implementations in other medication delivery and/or storage systems are also possible. In some implementations, such as in the illustrated example of FIG. 3A, the medication bladder 230 may include at least two exterior housing components configured to at least partially couple. The exterior housing components, when coupled, may be configured to form a bladder, pouch, or other enclosure for holding one or more fluids, such as a medication for administration to a wearer of a medication management system, including but not limited to a system such as described with reference to FIGS. 1A or 1B or another medication delivery system.

As illustrated in FIG. 3B, the at least two exterior housing components may include at least one second portion 232 and at least one first portion 231. The at least one second portion 232 may be configured to maintain shape under a threshold amount of pressure and the at least one first portion 231 may be configured to move, change shape, deflate, or otherwise modify its shape or orientation when exposed to at least a minimum threshold positive or negative pressure. The material of the second portion 232 may be a rigid material including, but not limited to, ABS, polypropylene (PP), polycarbonate (PC) or metal, or some combination thereof. The material of the first portion 231 may be a soft material including, but not limited to, thermoplastic elastomer (TPE), silicone, or some combination thereof. The first portion 231 may be in the form of a soft film of the selected material or materials. The interior surfaces of the second portion 232 and first portion 231 may be coated with an anti-aggregation coating including, but not limited to silicone dioxide or zinc oxide.

In some examples, the at least one second portion 232 may have at least one input port 2322 with at least one inlet seal 302 and at least one output port 2321. The at least one second portion 232 may include a geometry having at least one external surface 305, at least one internal surface 405, at least one wall 318, and at least one lip or edge 316. The geometry of the second portion 232 may be or include guiding structures such as curved surfaces or inclined planes, which may facilitate the movement of fluid to the output port or output ports 2321.

As shown in FIG. 3A, the exterior surface 305 of the second portion 232 may be on an exterior side of the enclosure formed by second portion 232 and first portion 231. The at least one external surface 305 of the second portion 232 may include at least one surface 307, and at least one structure 314. The surface 307 may form an approximately flat surface. The surface 307 may additionally or alternatively for an inclined surface. The inclined surface may tilt from an edge, such as edge 316, towards a central region 311.

In some examples, the at least one structure 314 may be configured to extrude, couple to, or otherwise extend from the at least one surface 307. The at least one structure 314 may be configured to provide a supporting structure for the second portion 232, improving the strength and/or rigidity of the second portion 232. In some examples, the at least one structure 314 may be configured to support one or more areas of the second portion 232 associated with at least one channel 2324 on an interior of the second portion 232, such as illustrated in FIG. 3C. In further examples, the channel 2324 may be embedded in the rigid structure to form a depression with respect to interior surface 405. The at least one structure 314 may be configured to additionally or alternatively provide support for other components of a second portion 232, such as an input port 2322 and/or output port 2321.

In some examples, the at least one structure 314 may include a plurality of structures, such as 6, 8, 10, or more structures or structural components. The structures 314 may protrude from the surface 307. The at least one structure 314 may be configured to extend from at least a portion of an edge 316 or wall 318 of the second portion 232 towards a central region 311 of the second portion 232, which may include an input port 2322 or other structure 301 surrounding and/or supporting an input port 2322, which may or may not form part of the at least one structure 314. The location of each structure of 314 on the surface 307 may correspond to the location of at least one channel 2324 of FIG. 3C.

The at least one structure 314 may vary in size and/or shape across the geometry of the second portion 232. For example, the at least one structure 314 may be configured to increase in height and/or change shape or profile between an edge 316 and/or wall 318 towards the central region 311. In some examples, the at least one structure 314 may be configured to have a different shape and/or more or fewer structures may be placed on the area of the second portion 232 based on a location of the second portion 232, such as, where the second portion 232 has an approximately rectangular top-down profile, nearer a corner 313, major edge 315, or minor edge 317.

A central region 311 may include at least one structure configured to extend perpendicularly from a major plane of the at least one external surface 305 of the second portion 232. The central region 311 may include a cavity or central depression 2323 on a bottom or interior side of a second portion 232, as illustrated in FIG. 3C. Advantageously, the form of the central region 311 may facilitate gathering and/or flow of medication from an interior of a medication bladder 230 towards an output region 319. As will be described herein, the second portion 232 may include one or more additional features that can help facilitate movement of medication towards this central region 311 and/or cavity 2323 of the central region 311, such as a plurality of channels 2324 on an interior surface 405, a contour or shape of the interior surface 405, a coating of the interior surface 405, and/or other features as described herein.

With continued reference to FIG. 3A, the central region 311 may include an input region 322 and/or an output region 319. The input region 322 may include an input port 2322 and/or associated inlet seal 302. The input port 2322 may be configured to have an opening at a center of the central region 311 such that the input port 2322 can be accessed from a top of the second portion 232. The inlet seal 302 may be configured to at least partially seal the input port 2322, as discussed herein.

The central region 311 may further include, in some implementations, at least a part of an output region 319, such as a lateral or otherwise oriented opening 321 of an output region 319. In some examples, the output region 319 may be offset from the central region 311 of the second portion 232. In some examples, the output region 319 may be oriented to output fluid at a lateral location, such as at, near, or above an edge 316. In some examples, the output region 319 may be configured to extend from a central region 311 towards an edge 316 of the second portion 232. However, other configurations are also possible. Additionally, or alternatively, the output region 319 could be located on a first portion 231 of the medication bladder 230. The output region 319 may additionally, or alternatively, protrude upward from the at least one surface 307 and/or bulk or planar portion of the second portion 232, such as illustrated in in FIG. 3A.

The output region 319 may be configured to facilitate evacuating medication from the medication bladder 230. The one or more output ports 2321 may serve as outlets to allow medication to exit the medication bladder. For example, the output region 319 may include one or more output ports 2321. An output port 2321 may facilitate the flow of medication by providing an opening through which the medication can leave the output region 319 and the medication bladder 230. The one or more output ports 2321 may include, but are not limited to a tube or cannula extending from a portion of the second portion 232, such as a lateral or otherwise oriented opening 321 in a central region 311 of the second portion 232. The one or more output ports 2321 may be composed of one or more materials, which may include, but is not limited to metal or silicone. In addition, the one or more output ports may contribute to facilitating the flow of medication through the output region 319 and out of the medication bladder 230.

In some examples, one or more output ports 2321 may be used together to increase the rate of the flow of medication through the output region 319 and out of the medication bladder 230. In some examples additional structures (e.g., channels, other types of protrusions, another structure, or some combination thereof) are used to direct the flow of medication through the one or more output ports to further facilitate the flow of medication through the output region 319 and out of the medication bladder 230. In some examples, a plurality of output ports 2321 of the one or more output ports may be used to direct the medication to different locations. As an example, a plurality of output ports 2321 of the one or more output ports 2321 may direct the medication to different injection points (e.g., locations on a patient's body configured for receipt of medication, such as by insertion of a needle). In some examples, at least one of the plurality of output ports 2321 may be configured to output fluid or medication from the medication bladder to a cannula or other fluid channel, such as a cannula 1164 described with reference to FIG. 1A. For example, an output port 2321 may be configured to couple to a cannula or other fluid channel that may pass through or be engaged with a pump configured to cause a flow of medication or fluid from the medication bladder 230 towards an injection point on the patient. The coupling of the cannula and output port 2321 may be a press fit or other connection type. In other examples, a cannula may be directly coupled to an output region 319 of the second portion 232.

Additionally, or alternatively, in examples utilizing a plurality of medication bladders 230 or a medication bladder 230 and at least one secondary fluid storage location, a plurality of output ports 2321 of the one or more output ports 2321 may be used to connect to additional medication bladders or fluid storage locations (e.g., connect an output port 2321 of one medication bladder 230 to the input port 2322 of another.) In further examples, the one or more output ports may be used to move medication between one or more medication bladders 230 or other fluid storage locations. For example, medication or other substance may be moved between the one or more medication bladders of a medication delivery system in order to deliver the medication to the patient as determined by a physician and/or by the medication delivery system. Advantageously, moving fluid between fluid storage locations, such as a medication bladder 230, may facilitate mixing or combining of medications or other substances prior to delivery to a patient.

As discussed, the second portion 232 may include an input region 322. The input region 322 may include an input or inlet port 2322. The input region 322 may be located at least in part at a central region 311 of the rigid portion. In some examples, the input region 322 may be offset from the center. However, other configurations are also possible. For example, the input region 322 may be located at an edge of the second portion 232. Additionally, or alternatively, the input region 322 could be located on a first portion 231 of the medication bladder 230. The input region 322 may additionally, or alternatively, protrude upward from a bulk or planar portion of the second portion 232, such as shown in FIG. 3A.

In some examples, the input region 322 may include one or more input ports 2322. The input port 2322 may serve as an inlet into the medication bladder 230. An inlet seal 302 may be coupled to the input port 2322. The input port 2322 may be configured to receive the inlet seal 302. The inlet seal 302 might be configured to allow a fill device, such as a needle, to pass through it to fill the medication bladder 230 with medication. The inlet seal 302 may be able to self-close after the fill device is removed. The material of the inlet seal 302 may be a soft material, such as silicone. Which may comprise sealable opening and/or be configured to receive the inlet seal 302, as described above with regards to the input port 2322. In some examples, the one or more input ports are used together to increase the rate of the flow of medication through the input region 322 and into the medication bladder 230. In some examples, additional structures (e.g., channels, other types of protrusions, another structure, a needle or needles, another fill device, or some combination thereof) may be used to help direct the flow of medication through the one or more input ports to further facilitate the flow of medication through the input region 322 and into the medication bladder 230. In some examples, the input port 2322 may align with the second opening 132 (discussed with reference to FIG. 1D) so that a fill device can access the input port 2322 from outside the disease management device 100.

FIG. 3C illustrates an example implementation of a rigid portion of an efficiently emptying medication bladder. With reference to FIG. 3C, the interior surface 405 of the second portion 232 may be configured to help facilitate efficient emptying of the medication bladder 230. For example, the interior surface 405 may be configured to include one or more features to reduce unwanted retention of fluid within the medication bladder 230 during emptying. For example, the interior surface 405 may include an anti-aggregation coating so as to reduce aggregation of fluid including chemical substances (e.g., medications, additives, and the like) stored in the medication bladder 230. The coating may partially and/or substantially reduce aggregation of fluid including chemical substances included in the medication bladder 230. In some examples, the coating may be hydrophobic. In some examples, the hydrophobic nature of the coating may prevent aggregation of fluid by rejecting the fluid from holding on to the surface of the coating.

As illustrated in FIG. 3C, the second portion 232 may have a plurality of channels 2324 configured to facilitate the flow of fluid out of the medication bladder 230. The plurality of channels 2324 may extend throughout the second portion 232. The plurality of channels 2324 may extend outward from a central region of the second portion 232 towards an edge 410 of the second portion 232. Additionally, or alternatively, the plurality of channels 2324 may extend outward from the output region 319 towards the edge 410 of the second portion 232. Additionally, or alternatively, the plurality of channels 2324 may extend outwards from the output port 2321. In some examples, the plurality of channels 2324 may extend outwards from the output region towards the edge of the second portion 232. The plurality of channels 2324 may extend outward towards an edge 316 of the at least second portion 232 radially from the central region 311 of the at least one second portion 232. However, other configurations and orientations are also possible.

The channels 2324 may approximately follow the contour or curvature of the second portion 232 on the interior surface 405. As discussed, the second portion 232 may include one or more guiding structures, such as curved or inclined surfaces. For example, the channels may be slightly curved towards a central cavity 2323, relatively flat in a central region 404, and more curved in a wall 318. As another example, the channels may be slightly curved towards a central cavity 2323, inclined in a central region 404 towards the wall 318, and more curved in a wall 318. The curved and/or inclined surfaces in the second portion 232 may facilitate the flow of fluid including chemical substances from the medication bladder. For example, the curvature of the curved portion (such as a wall 318) may be specified to mitigate the risk of fluid including chemical substances aggregating close to the edge 410 or the central cavity 2323. The curvature may apply a pressure to the one or more fluid including chemical substances in the medication bladder 230. The amount of pressure applied may depend on an amount of fluid including chemical substances in the bladder 230.

As referenced herein, the second portion 232 may be configured to couple to at least one first portion 231. In some examples, the at least one first portion 231 may be configured to change shape at least partially from a first shape or orientation into a second shape or orientation and/or any number of shapes or orientations between the first shape or orientation and the second shape or orientation. In some examples, the first shape may be of similar size and shape to a shape of the second portion 232. The second shape may be of similar size and shape to a general size and shape of the internal surface 405 of the second portion 232. However, the first shape and/or the second shape may not match or mirror the size and shape of the shape and/or surface(s) of the rigid portion. For example, the first and/or second shape may be smaller or larger than the size and shape of the rigid portion and/or include greater or fewer crevices, channels, or other components. In some implementations, the first shape may be approximately flat or planar and not configured to match the shape of the interior surface of the second portion 232.

In some implementations, the first shape of the first portion 231 may be oriented to mirror the orientation of the second portion 232 around a transverse plane or a point within a transverse plane formed by an edge 316, 317 at which the at least one second portion 232 and at least one first portion 231 meet or couple. The second shape may be configured to form an inverse orientation to the first shape and/or a parallel orientation to the at least one internal surface 405 of the second portion 232 when under negative pressure such that the second shape is configured to form an approximately parallel shape to the interior surface 405 of the second portion 232. As such, the medication bladder 230 may collapse so as to reduce an interior volume of the medication bladder 230, facilitating the emptying of the medication bladder 230 when desired. As noted above, different amounts of pressure (such as negative pressure), may be configured to reduce the interior volume of the medication bladder 230 different amounts and/or expel different amounts of fluid held in the interior volume of the medication bladder 230.

The second portion 232 may be coupled to the first portion 231. The second portion 232 and the first portion 231 may be coupled in such a way that they form at least a partially sealed enclosure. In some examples, the second portion 232 and the first portion 231 may be coupled at an edge 316 or other area by a process that may include a weld or overmolding. In some examples, the joining process used to couple second portion 232 and flexible portion may comprise a laser weld. Additionally, or alternatively, the joining process used may comprise an ultrasonic weld. Additionally, or alternatively, the joining process used may comprise an RF weld. Additionally, or alternatively, the joining process may comprise overmolding. However, other methods of coupling the second portion 232 and the first portion 231 are also possible. In some examples, an edge 316 may be created during the weld or other coupling or joining process. In other examples, the edge 316 may be created prior to and independently of a coupling or joining process.

The dimensions of the medication bladder 230 make it possible for the medication bladder 230 to fit into a minimally invasive device designed to comfortably sit on a person's body (e.g., on a person's abdomen, arm, etc.), such as the disease management system 1101 and/or the disease management system 1103, described in FIG. 1A and FIG. 1B. Additionally, the dimensions of the medication bladder 230 make it possible for the medication bladder 230 to include a volume of fluid including chemical substances sufficient to last for at least one or more days. For example, the medication bladder 230 may be configured to last two days, four days, 6 days, etc. The medication bladder 230 may even be configured to include an amount of fluid lasting for a week or more.

The size of the medication bladder 230 may be limited by the system into which its incorporated. For example, disease management system 1101 of FIG. 1A may be included inside of an enclosure. To ensure that other components of the disease management system 1101 also fit inside of the enclosure, the size of the medication bladder 230 may be limited to a specified range of dimensions. As another example, disease management system 1103 of FIG. 1B may be included inside of an enclosure. To ensure that other components of the disease management system 1101 also fit inside of the enclosure, the size of the medication bladder 230 may be limited to a specified range of dimensions. As a further example, a standard insulin delivery system may be inside of an enclosure. This may ensure that other components of the standard insulin delivery system fit inside the enclosure, the size of the medication bladder 230 may be limited to a specified range of dimensions.

In some examples, the volume of the medication bladder 230 may be approximately 3 mL, such as 2.7 mL. However, other volumes are also possible. For example, the volume may be greater than 2.7 mL. Alternatively, the volume may be smaller than 2.7 mL. Changes in volume of the medication bladder 230 may change the duration of usage between refilling the medication bladder 230. For example, a larger volume may increase the duration of usage between refills of the medication bladder 230.

### Pump System

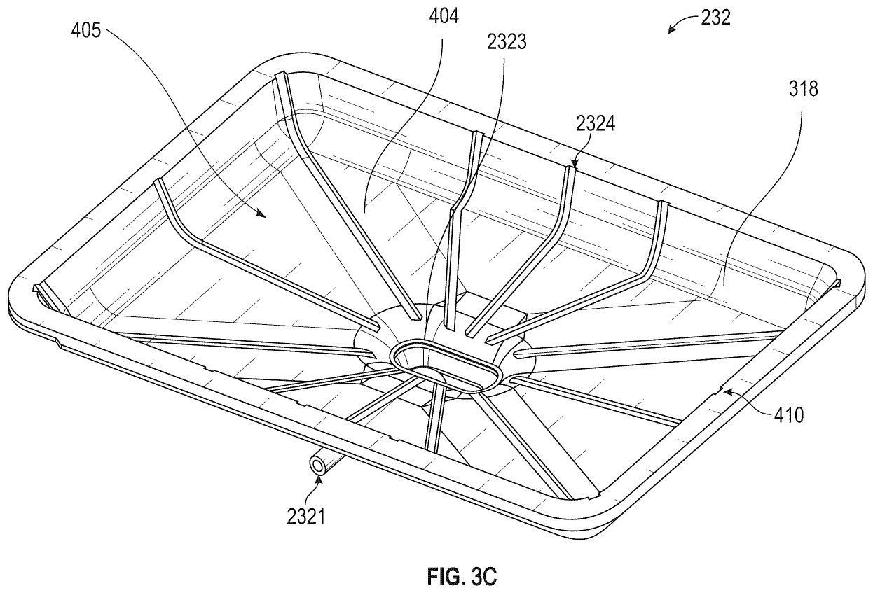

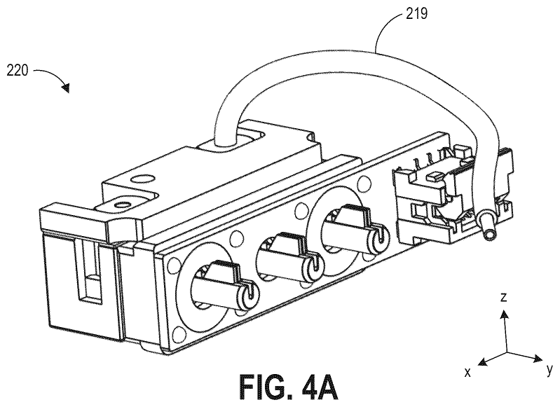

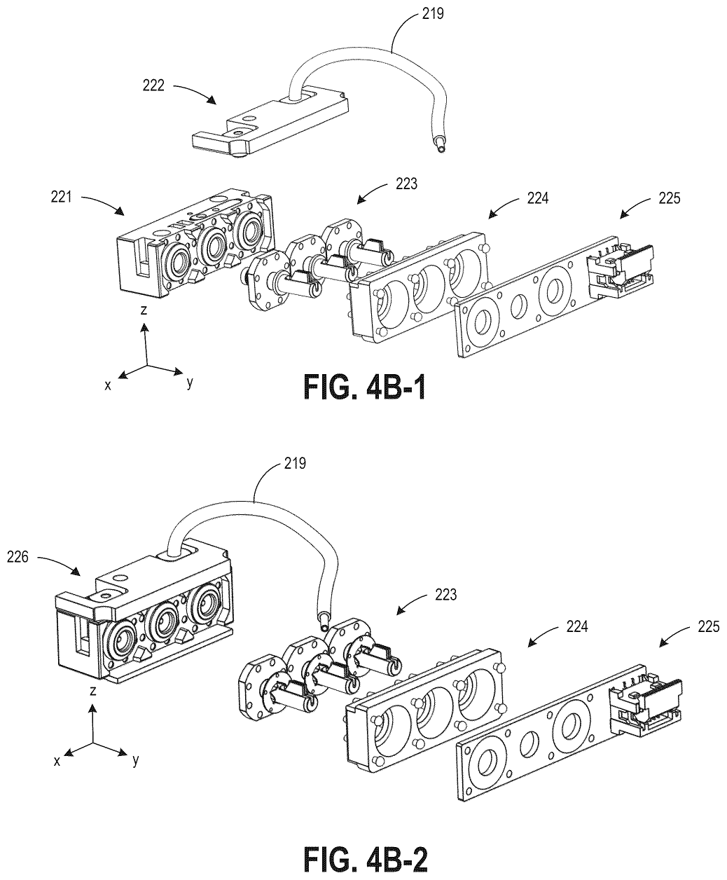

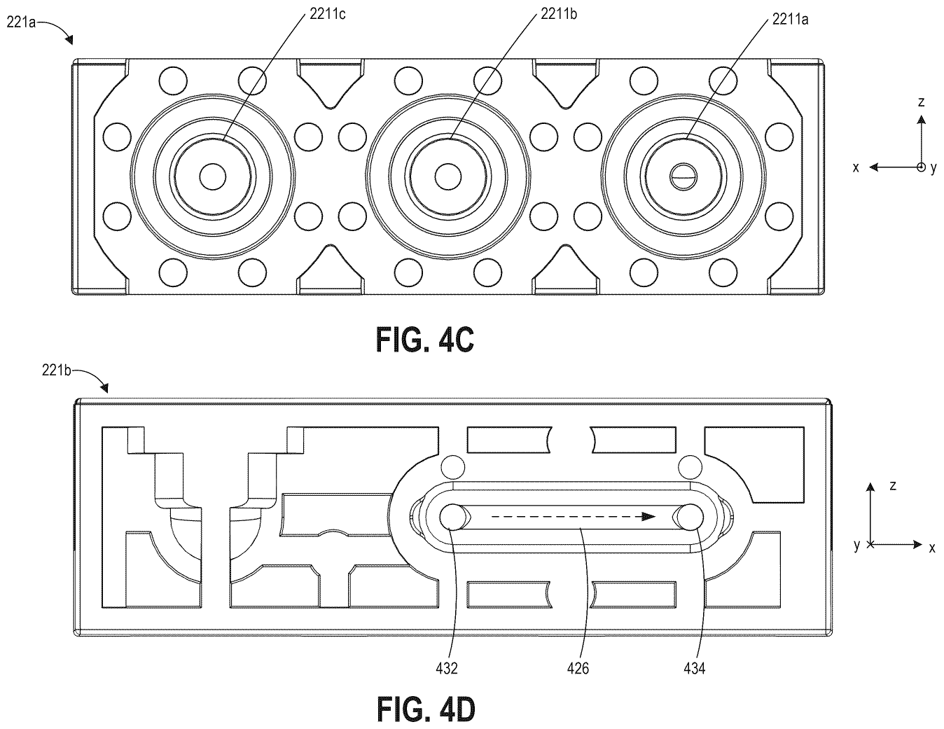

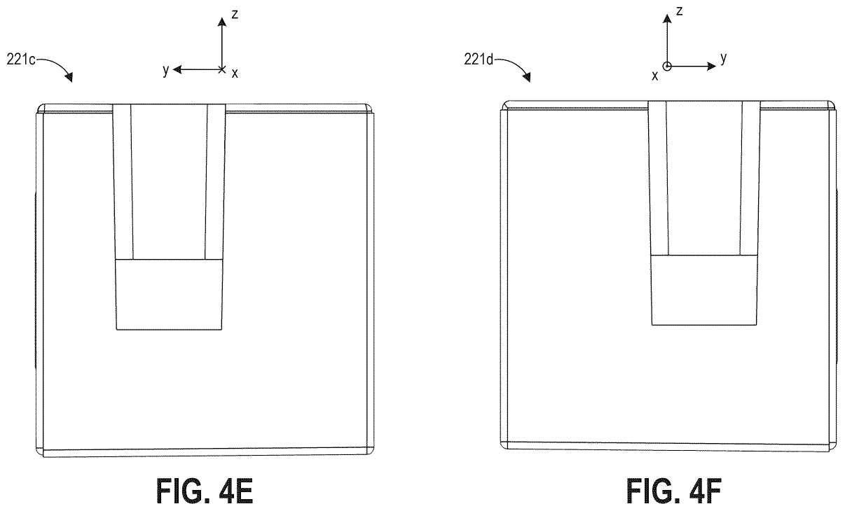

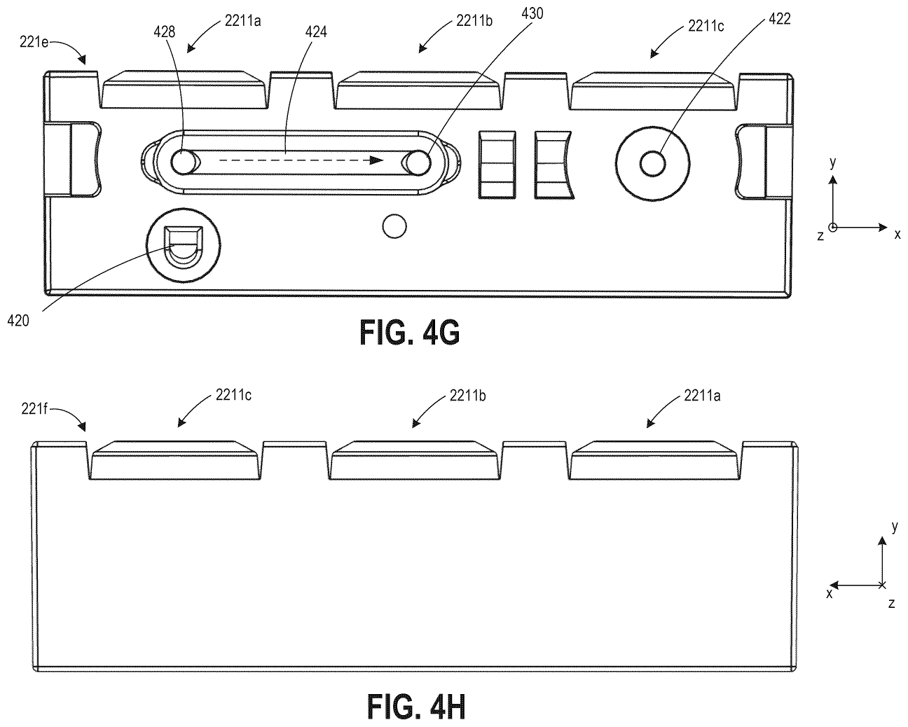

With reference to an illustrative example, FIG. 4A shows a perspective view of the pump system 220. In some aspects, the medication bladder 230 may be in communication with a pump system 220 (via a conduit 219, for example). As illustrated herein, aspects of an example blocker style pump (such as the pump system 220) may include muscle wire, for example shape memory alloy (SMA) wire. A muscle wire pump (such as the pump system 220) may use muscle wire to apply force and/or pressure to tubing or a flow path of medication. The pump system 220 can apply force and/or pressure to the tubing or flow path as a result of interaction between at least a spring and muscle wire. The pump system 220 may apply a force, such as a vacuum force and/or a negative pressure, to the medication bladder 230 via the conduit 219. This may cause the flexible portion (such as, the first portion 231 in FIG. 3A) of the medication bladder 230 to compress against the plurality of channels (such as, the plurality of channels 2324 in FIG. 3C) on a rigid portion (such as, the second portion 232 in FIG. 3A), which may facilitate the flow of the remaining fluid from the output port (such as, the output port 2321 in FIG. 3B). The interior surfaces of the medication bladder 230 may be coated in a material to mitigate aggregation of the fluid. This coating may further facilitate the flow of fluid from the medication bladder at least by reducing the medication remaining on the interior surfaces. Each of FIGS. 4A-4I include a three-dimensional axis key to help orient the various drawings relative to each other.

FIG. 4B-1 illustrates an exploded view of a first example pump system 220. In this example, the pump system 220 includes a pump assembly 221, a top assembly 222, a plunger assembly 223, a housing assembly 224, and a plate assembly 225. FIG. 4B-2 illustrates an exploded view of a second example pump system 220. In this example, the pump assembly 220 includes an integrated pump assembly 226, a plunger assembly 223, a housing assembly 224, and a plate assembly 225. The integrated pump assembly 226 can be a single-molded component including features of the pump assembly 221 and the top assembly 222 with reference to FIG. 4B-1. It is to be understood that additional and/or different pump component parts may be integrated.

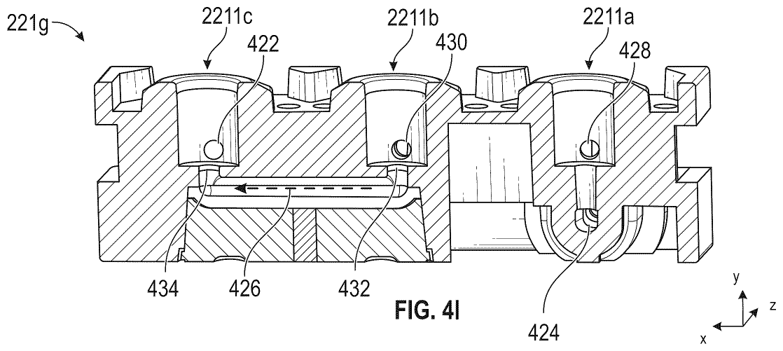

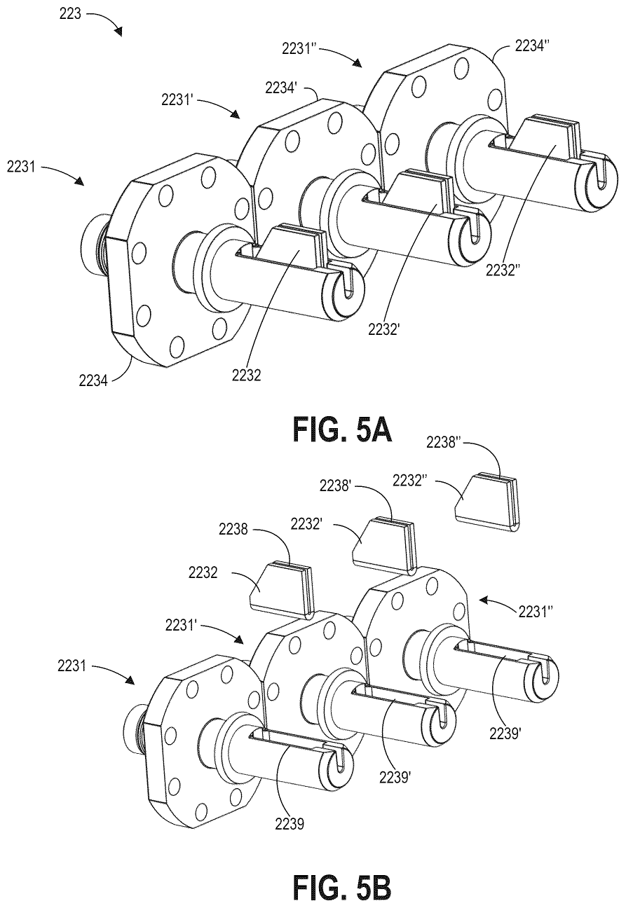

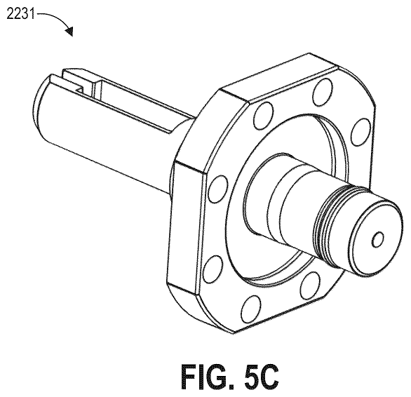

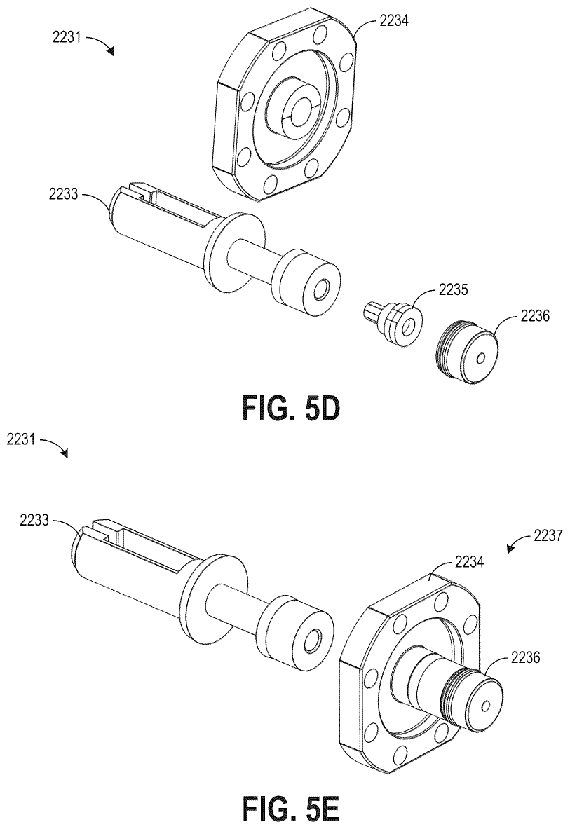

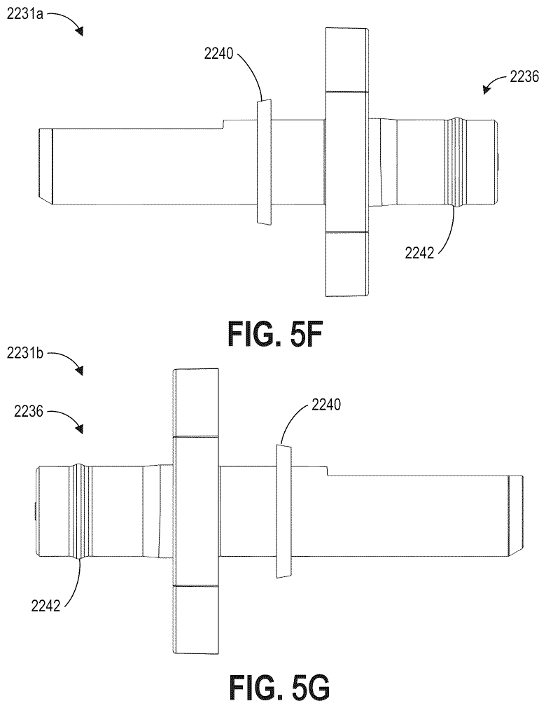

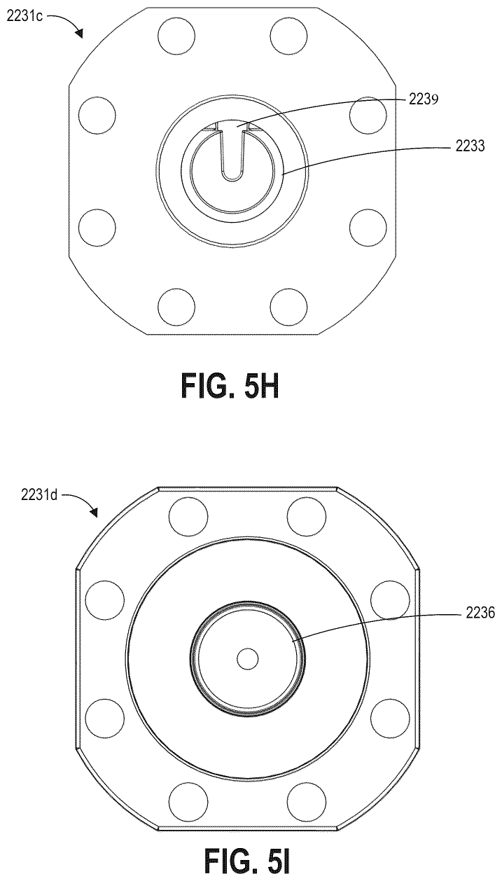

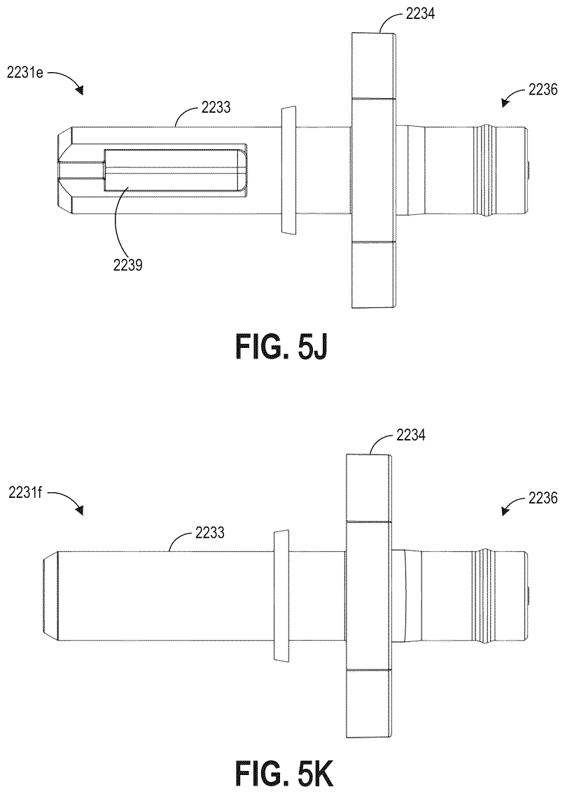

FIGS. 4C-4H illustrate various views of the pump assembly 221. FIG. 4C shows the pump assembly 221 from a first view 221a. FIG. 4D shows the pump assembly 221 from a second view 221b. FIG. 4E shows the pump assembly 221 from a third view 221c. FIG. 4F shows the pump assembly 221 from a fourth view 221d. FIG. 4G shows the pump assembly 221 from a fifth view 221e. FIG. 4H shows the pump assembly 221 from a sixth view 221f. FIG. 4I shows the pump assembly 221 from a cross-sectional view 221g. As illustrated in FIG. 4I the pump assembly 221 can include a plurality of cavities 2211a,b,c. The plurality of cavities 2211a,b,c may each receive a plunger (such as from the plunger assembly 223 as illustrated in FIG. 5A).

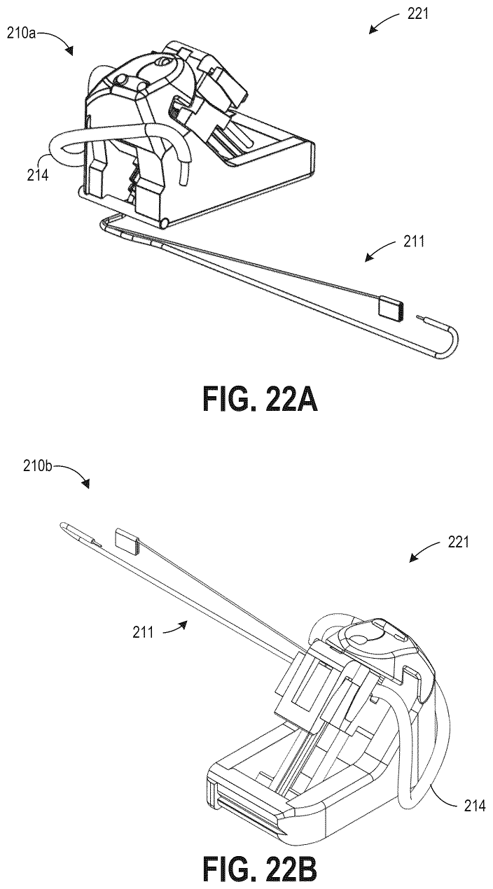

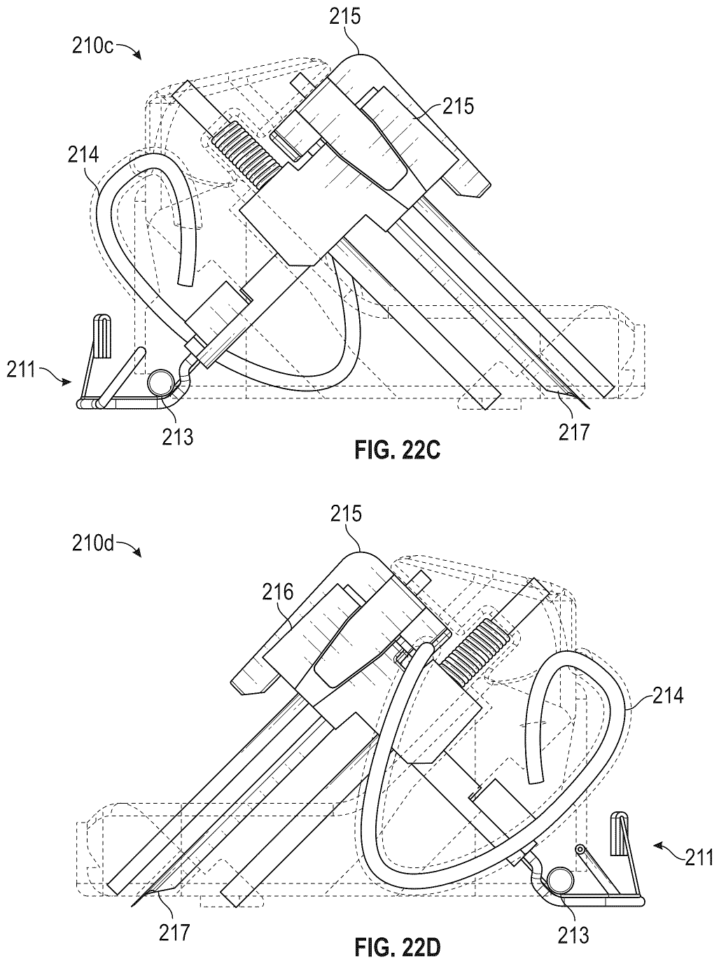

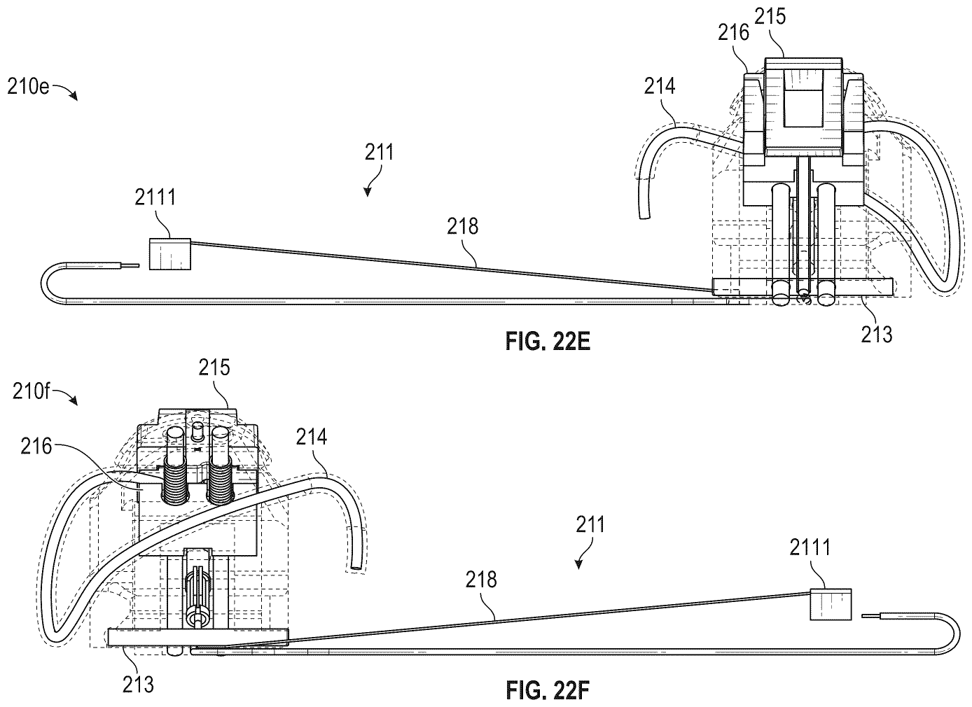

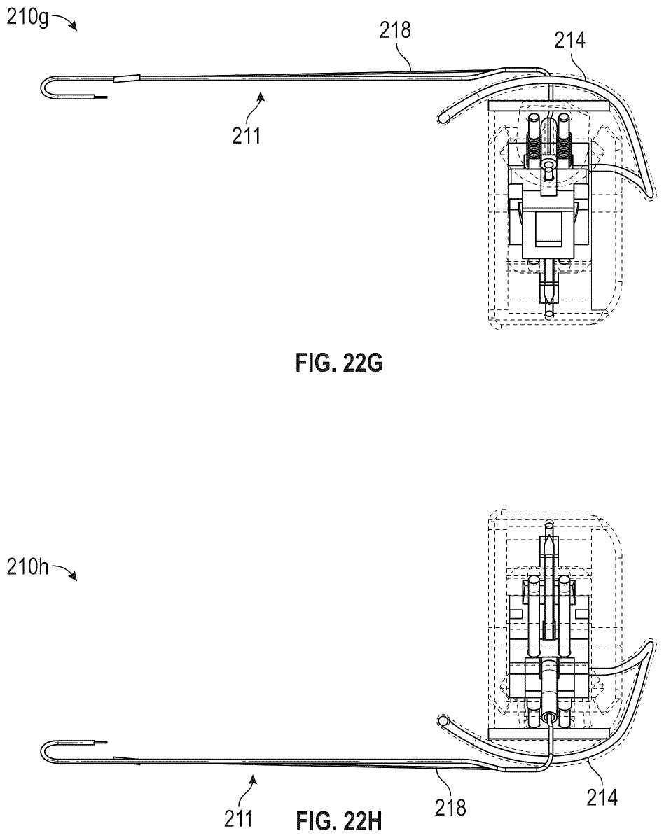

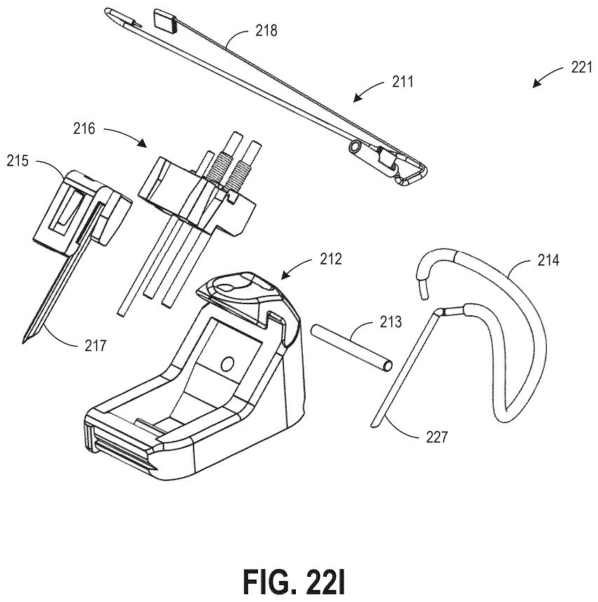

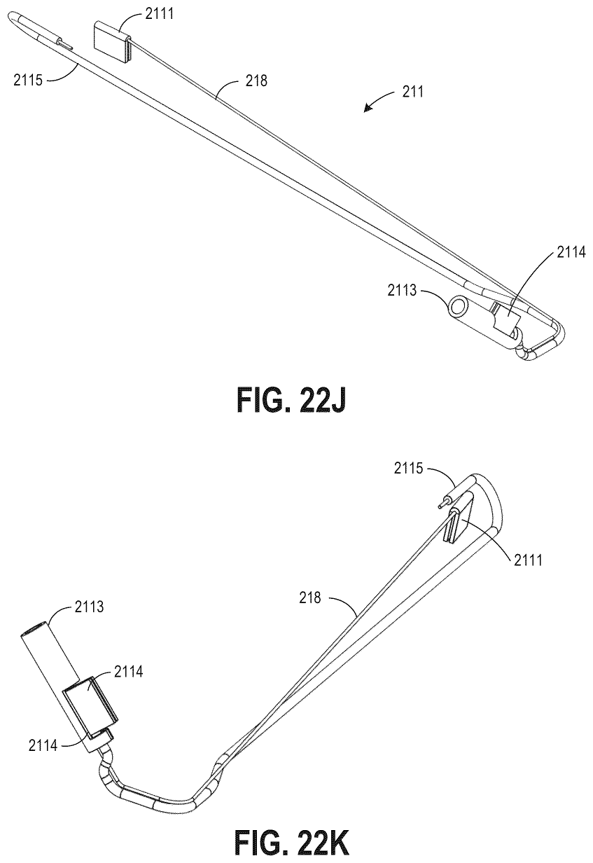

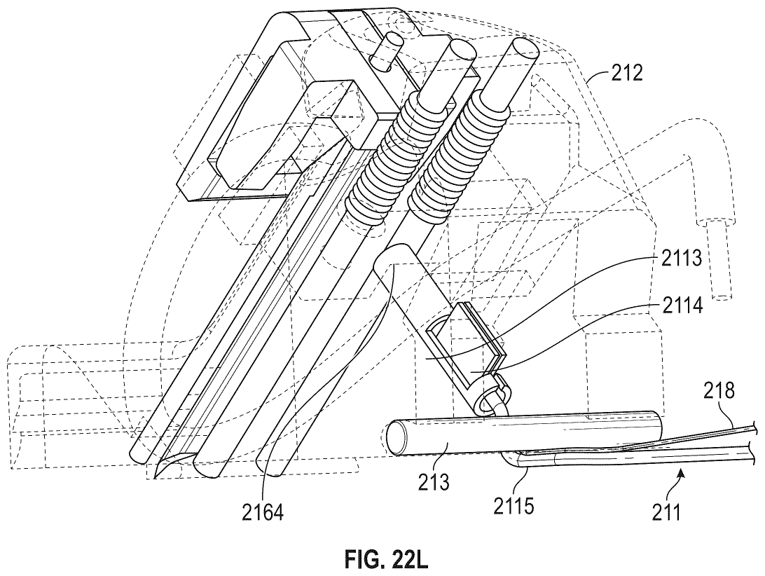

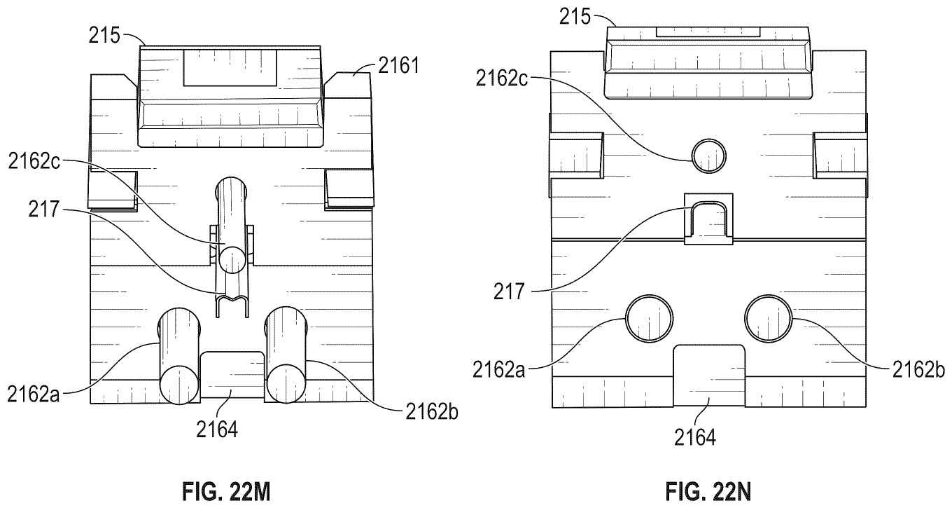

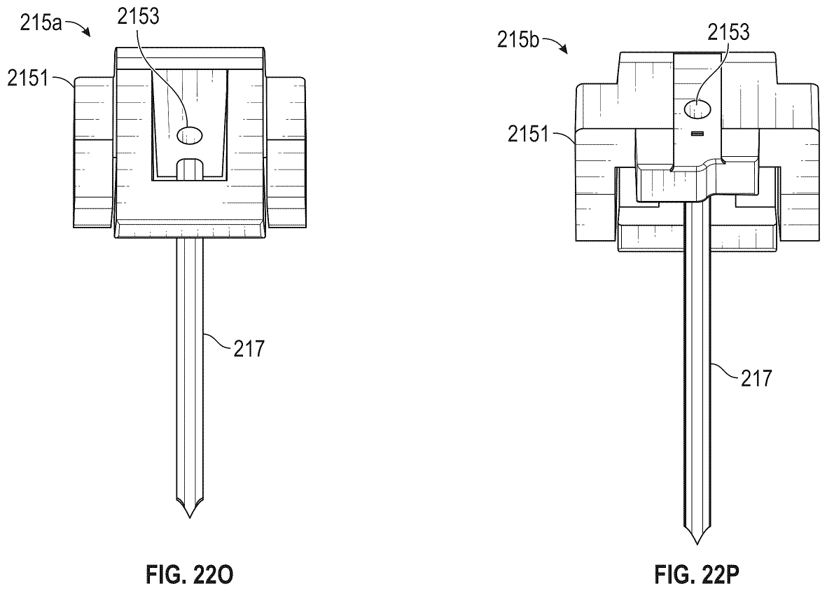

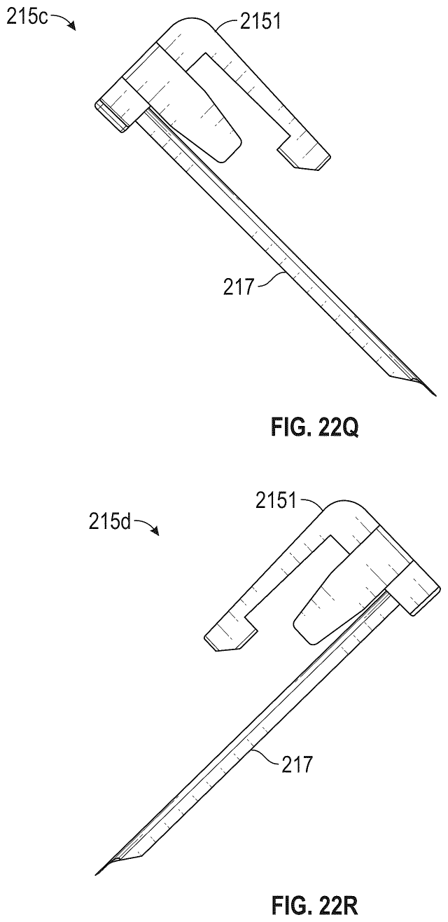

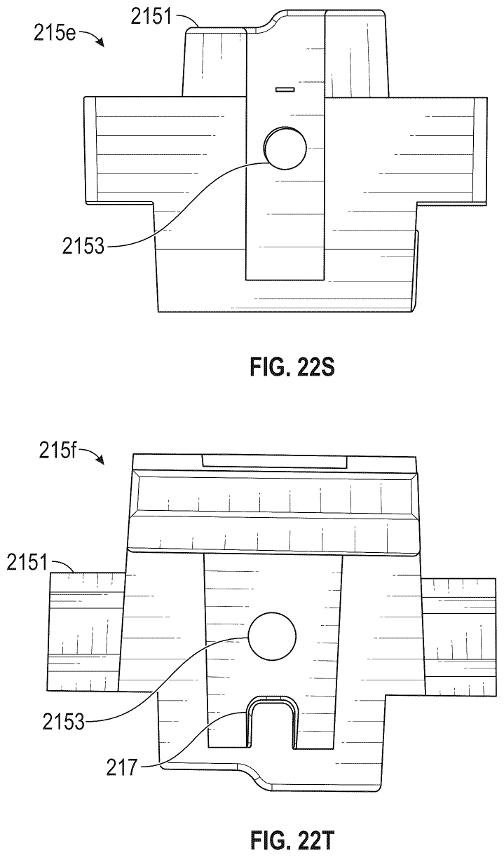

With reference to FIGS. 4D, 4G, and 4I, the pump assembly 221 includes an inlet port 420, a flow path 424, a flow path 426, and an outlet port 422. Fluid (e.g., fluid medication) from the medication bladder 230 can flow to the inlet port 420 via the conduit 219. The fluid can then flow from the inlet port 420 to the cavity 2211a. The fluid can flow from the cavity 2211a to the cavity 2211b via flow path 424 in the direction indicated by the dashed arrow in FIG. 4G. The flow path 424 can have a flow path entry 428 and a flow path exit 430. From the cavity 2211b, the fluid can flow to the cavity 2211c via the flow path 426 in the direction indicated by the dashed arrow in FIG. 4I. The flow path 426 can include a flow path entry 432 and a flow path exit 434. The fluid can flow from 2211c to the outlet port 422. From the outlet port 422 the fluid may flow along a conduit or tube (e.g., the conduit 214 discussed with reference to FIG. 22I) to a cannula (e.g., the cannula 227 discussed with reference to FIG. 22I) for administration to the user. Flow along the described path can be caused by actuation of the plungers 2231, 2231′, 2231″ within their respective cavities 2211a, 2211b, and 2211c.

For example, the pump assembly 221 (and components thereof) may be similar or identical to and/or incorporate any of the features described and/or illustrated with respect to any of the devices, assemblies, systems, and/or methods described in U.S. application Ser. No. 17/817613, which was filed on Aug. 4, 2022 and is titled “MEDICATION DELIVERY PUMP FOR REDUNDANT STAGGERED GLUCOSE SENSOR INSULIN DOSAGE SYSTEM,” and U.S. application Ser. No. 18/605630, which was filed Mar. 14, 2024 and is titled “MODULAR DISEASE MANAGEMENT DEVICE AND AUTOMATED NEEDLE AND CANNULA INSERTION DEVICE,” the disclosures of which hare expressly incorporated by reference herein in their entireties for all purposes.

FIG. 5A shows a perspective view of the plunger assembly 223. The plunger assembly 223 can include a first plunger 2231, a second plunger 2231′, a third plunger 2231″, a first guide component 2232, a second guide component 2232', and a third guide component 2232″. Each plunger of the plunger assembly 223 may adjust a position (e.g., by actuation of a muscle wire) to cause compression of content (such as medication) of a flow path (such as cavities 2211a,b,c discussed with reference to FIGS. 4C-4I) that can carry medication from a medication pouch (such as medication bladder 230 of FIG. 3A) to a patient. However, other configurations of a plunger and medication delivery or flow path are also possible.

Now with reference to FIG. 4A-5L, in some examples, each of plungers 2231, 2231′, and 2231″ can affect fluid within the flow path. As the plunger 2231, 2231′, or 2231″ returns to a relaxed position (e.g., when the muscle wire is no longer actuated and the plate component 2234—also referred to herein as disc spring 2234—acts to oppose contraction of the muscle wire), the plunger 2231, 2231′, or 2231″ may cease to exert negative pressure and/or begin to exert positive pressure, which may drive some fluid further along the fluid flow path. In some such examples, actuation and movement of the plungers 2231, 2231′, and 2231″ with their respective disc springs 2234, 2234′, and 2234″ exerts the pressure. That is to say, the movement of the plungers 2231, 2231′, and 2231″ with their respective disc springs 2234, 2234′, and 2234″ changes the volume available to the fluid medication within the cavities 2211a, 2211b, and 2211c, which causes a change in pressure exerted on the fluid medication. In other such examples, the plungers 2231, 2231′, and 2231″ can create a seal (e.g., using the ridges 2242) against the interior surface of the cavities 2211a, 2211b, and 2211c, and movement of the plungers 2231, 2231′, and 2231″ can in turn cause movement of the position of the seal, which causes exertion of pressure on the flow path. In other words, the movement of the seal between the plungers 2231, 2231′, and 2231″ and the cavities 2211a, 2211b, and 2211c changes the volume available to the fluid medication within the cavities 2211a, 2211b, and 2211c, which causes a change in pressure exerted on the fluid medication. In some examples, the plunger 2231 may be configured to seal or block or at least partially seal or block a portion of the tube or flow path. In some examples, each of the plungers 2231, 2231′, and 2231″ can, in an unactuated position, block the flow path of fluid medication (e.g., blocking one or more of the flow path 424, the flow path entry 428, the flow path exit 430, the flow path exit 434, and/or the outlet port 422). In such examples, the medication bladder 230 may be under a positive pressure, such that when the plungers 2231, 2231′, and 2231″ are actuated and cease blocking the flow path of fluid medication, fluid medication flows from the medication bladder 230, through the pump system 220, and to the cannula 227. In other examples, the plunger 2231 may not completely block the flow path of medication in the tube (such as within cavity 2211a in FIG. 4I). In some such examples, the plunger 2231 may not block the flow path at all. In some examples, the plunger 2231 may occupy only a portion of the cavity (e.g., cavity 2211a, 2211b, or 2211c). In such examples, the plunger 2231 can effectuate motion of fluid in the flow path solely by exerting negative pressure when moving to an actuated state and exert positive pressure when returning to the relaxed state from the actuated state. In these examples, fluid (e.g., fluid medication) may be capable of flowing past the plunger 2231 in either the actuated state or the relaxed state.

A benefit of the pump system 220 is that, with the three separate plungers 2231, 2231′, and 2231″ of the plunger assembly 223, no directional valves are needed. The plungers 2231, 2231′, and 2231″ can be sequentially actuated to “step” small volumes of liquid medication from the drug reservoir, through the flow path, to a cannula. The sequential actuation of the plungers 2231, 2231′, and 2231″ controls the flow direction of the small volume of liquid medication. In some examples, the pump system 220 includes no directional valves. In some examples, the device 100 includes no directional valves.

Another benefit of the pump system 220 is the precision of medication delivery. The series of plungers (e.g., plungers 2231, 2231′, and 2231″) can “step” out small, precise volumes. The precision and repeatability of these delivered volumes is sufficient that, in some examples, the device 100 does not include sensors to verify that the correct volume is delivered. In some examples, the actuation of the plungers (e.g., plungers 2231, 2231′, and 2231″) can be sufficiently precise that the device 100 does not include sensors to verify pressure within the fluid flow path (e.g., within or along the conduit 219, the flow path 424, the flow path 426, and/or the conduit 214, among other potential locations).

In some examples, using the plungers 2231, 2231′, and 2231″, the pump system 220 can provide a minimum volume (e.g., from a single actuation of the plungers 2231, 2231′, and 2231″, also referred to as a minimum bolus volume) of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100μL, or a volume within any two of the previous values, though in some instances other values may suitably be implemented. In some examples, a single actuation of plungers 2231, 2231′, and 2231″ can provide a volume of 0.1-10μL to a user.

In some examples, the plate components 2234, 2234′, and 2234″ can be integrated into a single plate component coupled to the plunger component 2233 of each of the respective plungers 2231, 2231′, and 2231″. In some examples, the plate component 2234 can provide a seam between an interior of the pump (e.g., an interior of the cavity 2211 with reference to FIG. 4I) and an exterior of the pump. In such examples, the plate component 2234 can prevent leakage of the liquid medication from the pump.

The guide components 2232, 2232′, 2232″ can each include a cavity 2238, 2238', or 2238″ which can receive an end of the one of the muscle wires. The muscle wires can be secured within each of the cavities 2238, 2238', or 2238″ with an adhesive. In some such examples, the guide components 2232, 2232′, 2232″ do not act as a crimp. Each of the guide components 2232, 2232′, 2232″ can be positioned within a corresponding slot 2239, 2239′, or 2239″. In some examples, the guide components 2232, 2232′, 2232″ can fit tightly within the slots 2239, 2239′, or 2239″. In this way, the guide components 2232, 2232′, and 2232″ can secure a muscle wire to one of plunger 2231, 2231′, or 2231″, respectively.

In some examples, the plunger 2231 may include a material, such as silicone. In some examples, the plunger 2231 may be configured to form a seal when pressed into the flow path in the tube. The plunger 2231 may include overmolded components as discussed with reference to FIG. 5E. For example, the plunger head 2236 may be overmolded on to the plunger component 2233. The plate component 2234 may be overmolded onto the plunger 2231. In some further example, the plate component 2234 and plunger head 2236 can form a single integrated overmolded component. For example, the overmold may include silicone or a material of the like. The plunger 2231 may include a seal (e.g., O-ring seal) to maximize the formation of the seal formed when pressed into the flow path. In this way, the plunger 2231 can reduce the amount of fluid that may escape from the flow path. A shape of the plunger 2231 may be any suitable shape to block a flow path directly or indirectly. For example, the plunger 2231 may be configured to compress a tube or flow path, directly block a flow path of the medication or the like. In some examples, a contact point of the plunger 2231, such as at least one surface of the plunger 2231 configured to engage directly or indirectly with the flow path of the medication, may be rectangular, square, circular, elliptical, oblong, asymmetrical, or symmetrical. The plunger 2231 may be configured to be engaged by one or more springs (e.g., the plate component 2234 acting as a disc spring) such that the one or more springs can provide force to the plunger 2231 to return the 2231 to a relaxed position. In some examples, in the relaxed position, the plunger 2231 can block or partially block the flow path. In some examples, the one or more springs may apply a retraction pressure to the plunger 2231 to interrupt the flow path. In other words, the springs may oppose retraction of the muscle wires.

FIG. 5B shows a perspective view of the plunger assembly 223 with the guide components 2232, 2232′, 2232″ separated from the first plunger 2231, second plunger 2231′, and third plunger 2231″, respectively. In some examples, the guide components 2232, 2232′, 2232″ may each include a cavity to allow wire to pass. FIG. 5C shows a perspective view of the first plunger 2231. FIG. 5D shows an exploded view of an example first plunger 2231. In some examples, the plunger 2231 can include a plunger component 2233, a plate component 2234, an adapter component 2235, and a plunger head 2236. The plate component 2234 can include a pliable material. In such examples, the 2234 can act as a spring (e.g., a disc spring). The plate component 2234 can act to exert force to return the plunger 2231 to a neutral, non-actuated position (also referred to herein as a relaxed position) when actuation of the muscle wire ceases. FIG. 5E shows an exploded view of a second example of the first plunger 2231. In the example shown in FIG. 5E, the plunger 2231 can include a plunger component 2233 and a sheath component 2237. The sheath component 2237 can be a single, integrated piece that includes a plate component 2234 and a plunger head 2236. In some examples, the sheath component 2237 can be overmolded onto the plunger component 2233. It is to be understood that either of the example plunger 2231 shown in FIG. 5D or FIG. 5E can be suitably implemented.

FIGS. 5F-5K illustrate various views of the plunger assembly 223. FIG. 5F shows the plunger assembly 223 from a first view 2231a. FIG. 5G shows the plunger assembly 223 from a second view 2231b. FIG. 5H shows the plunger assembly 223 from a third view 2231c. FIG. 5I shows the plunger assembly 223 from a fourth view 2231d. FIG. 5J shows the plunger assembly 223 from a fifth view 2231e. FIG. 5K shows the plunger assembly 223 from a sixth view 2231f. FIG. 5L shows the first plunger 2231 from a cross-section view 2231g. Though FIGS. 5C-5L are discussed with reference to the first plunger 2231, it is understood that any of the discussed features may be incorporated into either or both of second and third plungers 2231′, 2231″.

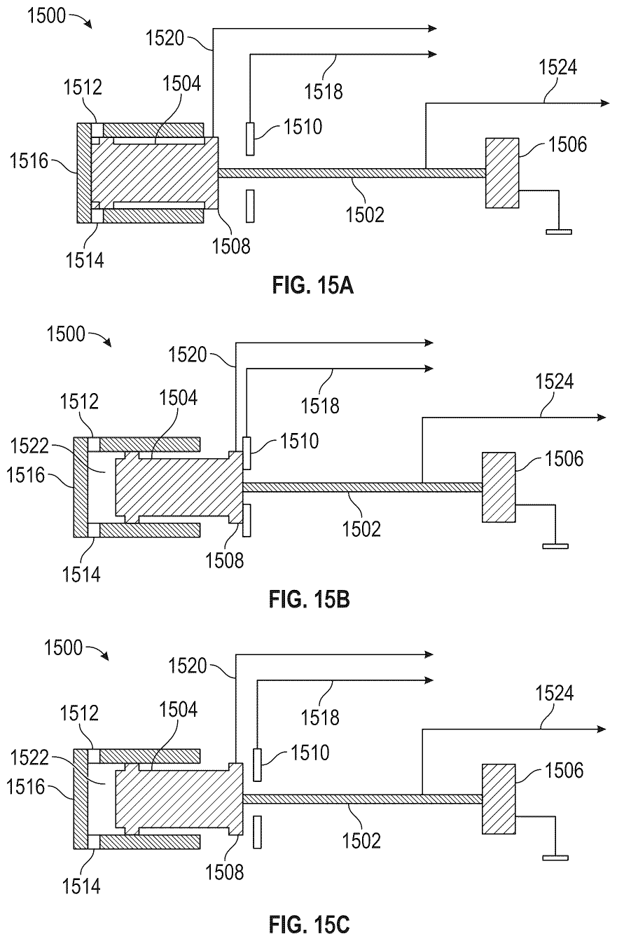

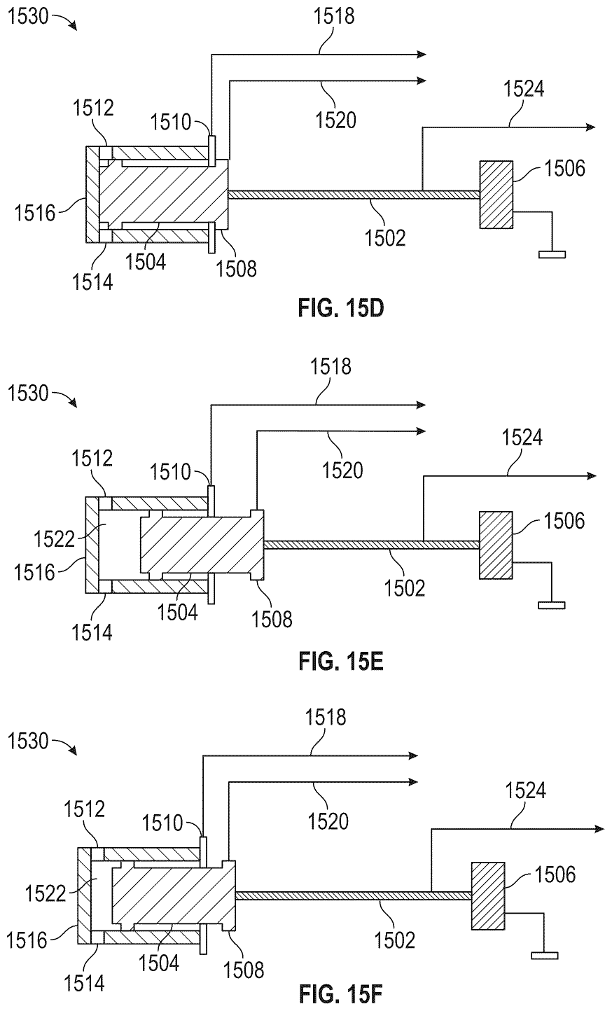

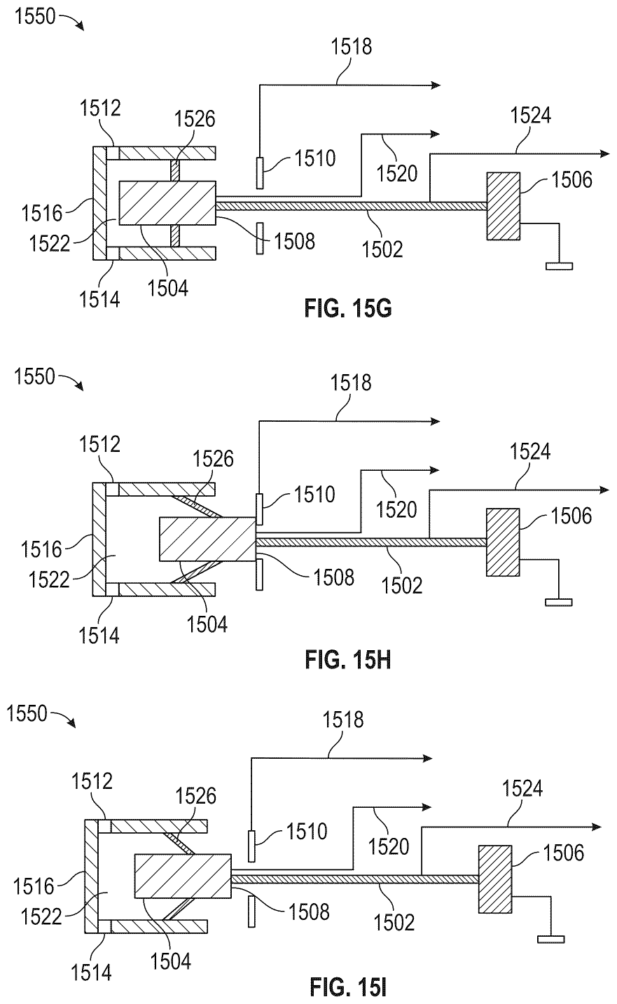

The plunger 2231 can include a flange 2240. In some examples, the flange 2240 can couple to a component capable of being sensed by an electrode (for example, for sensing by the stationary electro-mechanical contact 1510 discussed with reference to FIGS. 15A-15C). In alternative examples, the flange 2240 itself is capable of being sensed by an electrode (for example, for sensing by the stationary electro-mechanical contact 1510 discussed with reference to FIGS. 15A-15C). The plunger head 2236 of the plunger 2231 can include ridges 2242. In some examples, the ridges 2242 do not contact and/or form a seal with an interior cavity of the pump assembly 221. In other examples, the ridges 2242 may contact and/or form a seal with an interior cavity of the pump assembly 221 (e.g., ridges 2242 may contact and/or form a seal with an interior surface of one of the cavities 2211a, 2211b, or 2211c).

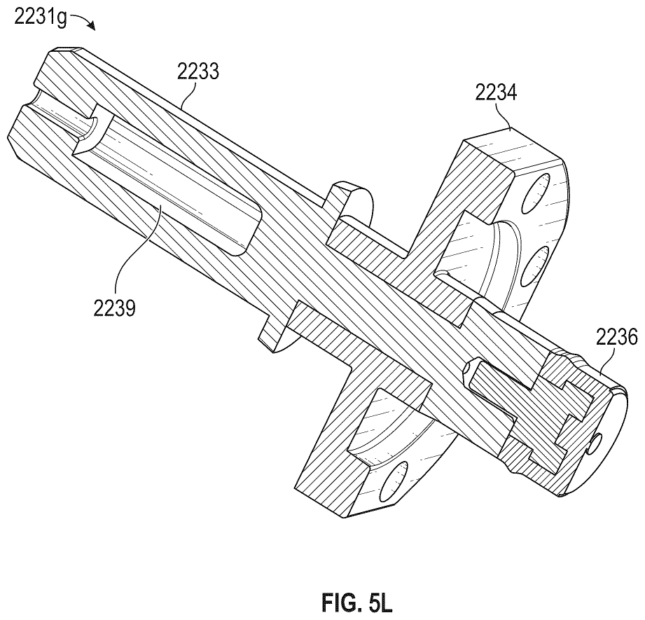

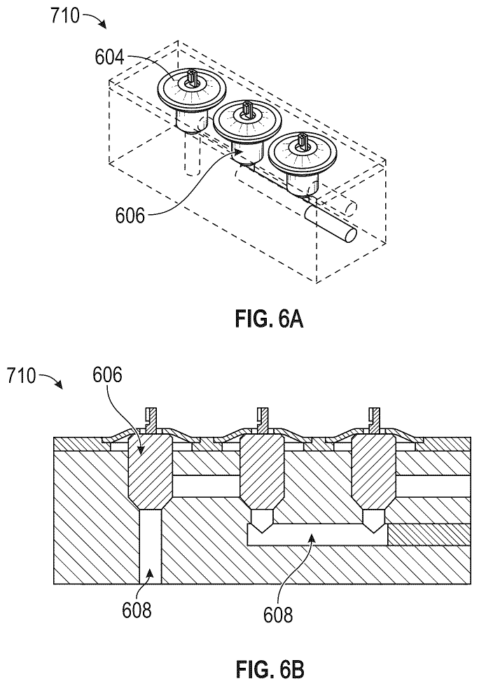

General operating principles of a muscle wire pump assembly are now discussed with reference to FIGS. 6A and 6B. A spring of a muscle wire pump assembly 710 may include a disc spring 604. In some examples, a disc spring 604 may include silicone or other polymer, such as thermal plastic elastomer (TPE), thermal plastic polyurethane (TPU). Other materials may also be used that are capable of providing a sufficient restoring force in response to displacement of at least a portion of the disc spring to move a plunger coupled to the muscle wire. In some examples, the disc spring may include at least a portion configured to be displaced that has an approximately frusto-conical shape or the like. In some examples, the disc spring may be approximately flat when not displaced. In some examples, the disc spring may be approximately frusto-conical when displaced. Other shapes of the disc spring may also be possible. For example, the disc spring may have an approximately circular top-down profile with a curved side profile. In some examples, the curved side profile may include an S-shaped curve. However, other curves of a curved side profile are also possible.

A plunger 606 (e.g., the plunger 2231) can be configured to travel within a volume in liquid communication with a flow path for carrying medication from a medication pouch to a patient. For example, as illustrated in FIGS. 6A and 6B, a plunger 606 may be configured to block a flow path 608 of medication. In some examples, the plunger 606 may be configured to seal or block or at least partially seal or block a portion of the tube or flow path 608. In other examples, the plunger 606 may not block the flow path 608, but nonetheless may be capable of exerting positive or negative pressure on the flow path 608. In some such examples, the plunger 606 may include a material, such as silicone, configured to form a seal when pressed into the flow path in the tube. The plunger 606 may include overmolding. For example, the overmold may include silicone or a material of the like. The plunger 606 may include a seal (e.g., O-ring seal) to maximize the formation of the seal formed when pressed into the flow path. This can reduce the amount of fluid that may escape from the flow path. A shape of the plunger 606 may be any suitable shape to block a flow path directly or indirectly. For example, a plunger 606 may be configured to compress a tube or flow path, directly block a flow path of the medication or the like. In some examples, a contact point of the plunger, such as at least one surface of the plunger 606 configured to engage directly or indirectly with the flow path of the medication, may be rectangular, square, circular, elliptical, oblong, asymmetrical, or symmetrical. A plunger 606 may be configured to be engaged by the one or more springs 604 such that the one or more springs 604 can provide pressure to the plungers 606 in order to block or partially block the flow path. In some examples, the one or more springs 604 may apply a retraction pressure to the plungers 606 to interrupt the flow path. In other words, the springs 604 may retract from the muscle wires to allow the plungers 606 to interrupt the flow path.

In alternative examples, the plunger 606 need not form a seal. In such examples, a seal may be provided by another component, for example a disc spring 604. Movement of the plunger 606 within the assembly 710 can exert pressure within the flow path 608.

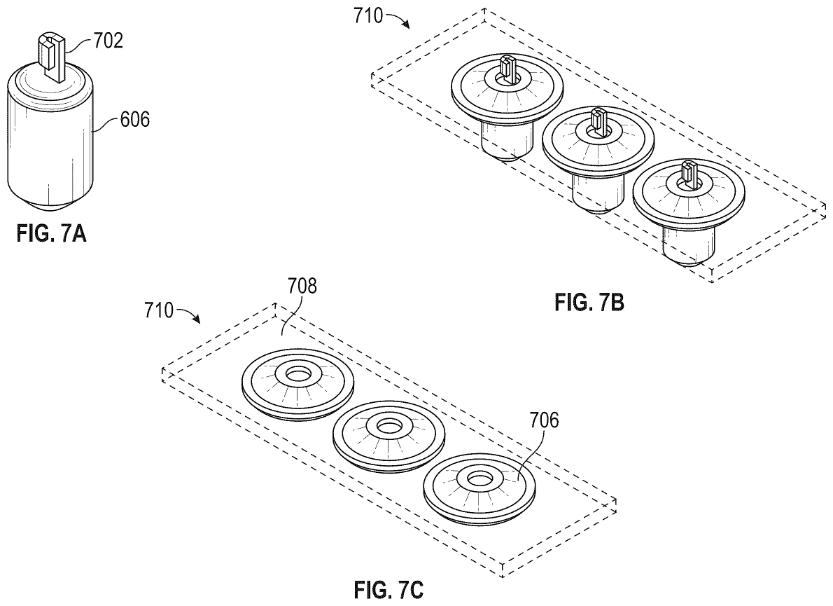

With reference to FIGS. 7A and 7B, in some examples, the plungers 606 may be connected to or include a coupling 702. In some examples, the 702 include the guide components 2232 and slot 2239 discussed with reference to FIGS. 5A and 5B. The coupling 702 can connect the plunger 606 to a muscle wire, thereby allowing contraction of the muscle wire to actuate the coupling 702. The muscle wire can actuate the coupling 702 configured to contract when an electrical current is applied to the muscle wire and/or the muscle wire is heated. The coupling 702 may lift the plunger from the area of the flow path of plate 708, causing flow of medication from the medication pouch to the patient.

With reference to FIG. 7C, one or more components of a muscle wire pump may be overmolded and/or chemically bonded together as part of a manufacturing process. In some examples, the one or more disc springs 604, 706 may be bonded and/or overmolded to the plunger 606. In some examples, the one or more disc springs 604, 706 can be bonded to a plate 708 to form an assembly 710 with the plungers 606, wherein the plate 708 is configured to include cavities to allow one or more plungers 606 to move at least a portion of the plunger in a vertical direction with respect to a horizontal plane of the plate 708. The one or more disc springs 706 can provide a retraction pressure to return the plungers 606 to an unactuated position (also referred to herein as a relaxed position). In some examples, the plunger 606 interrupts the flow path in the unactuated position. In some examples, the disc springs 706 may include a membrane configured to encase the spring. For example, the membrane may include silicon, or the like. In other examples, the one or more disc springs 706 includes a single pliable material.

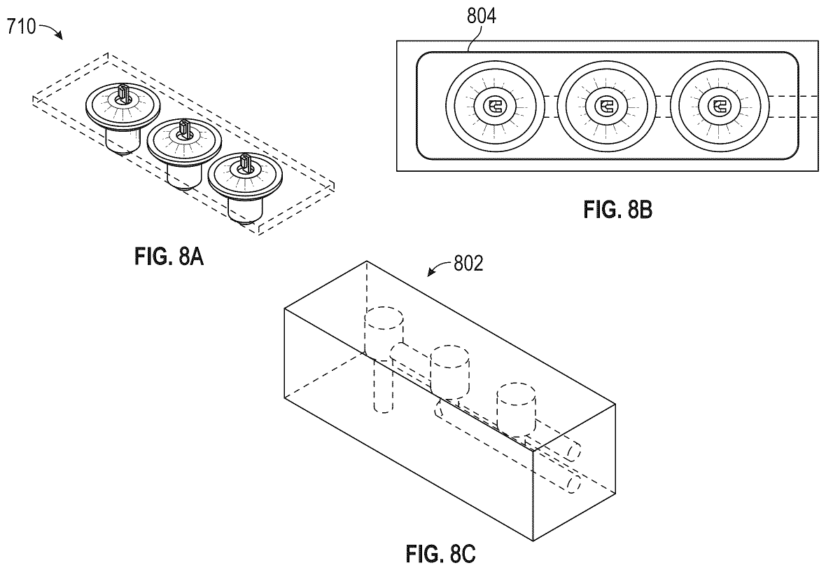

As shown in FIGS. 8A-8C, an assembly 710 may be configured to engage with a bottom portion 802 of a pump that includes the flow path of medication. The assembly 710 may be configured to be permanently or semi-permanently coupled to the bottom portion 802. The assembly 710 may include a surface 804. As illustrated in FIGS. 8A-8C, an assembly may be integrated into a larger assembly or configured to couple the pump assembly into a medication delivery device, such as a disease management device illustrated in FIGS. 1C-1B.

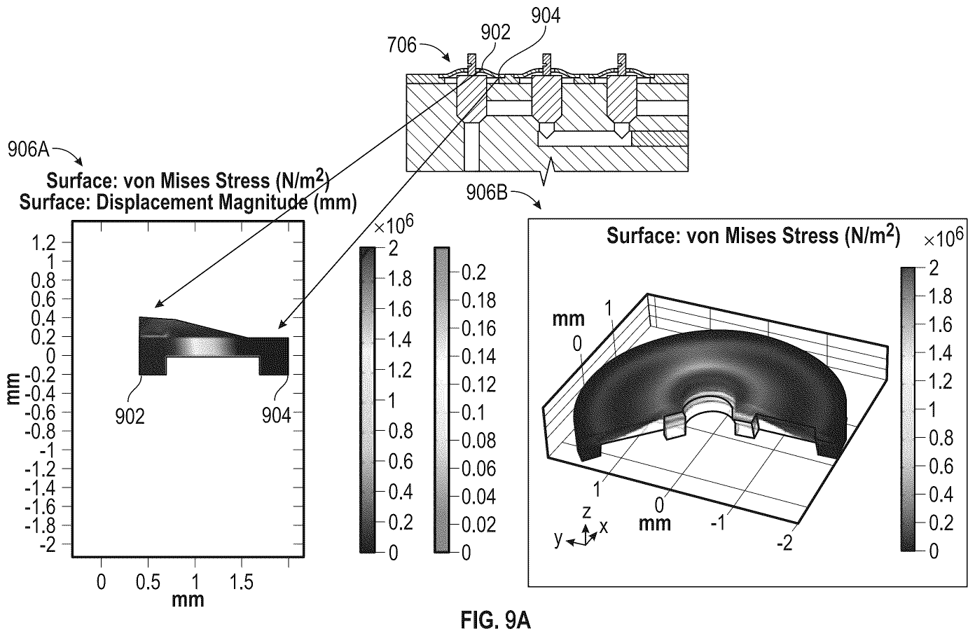

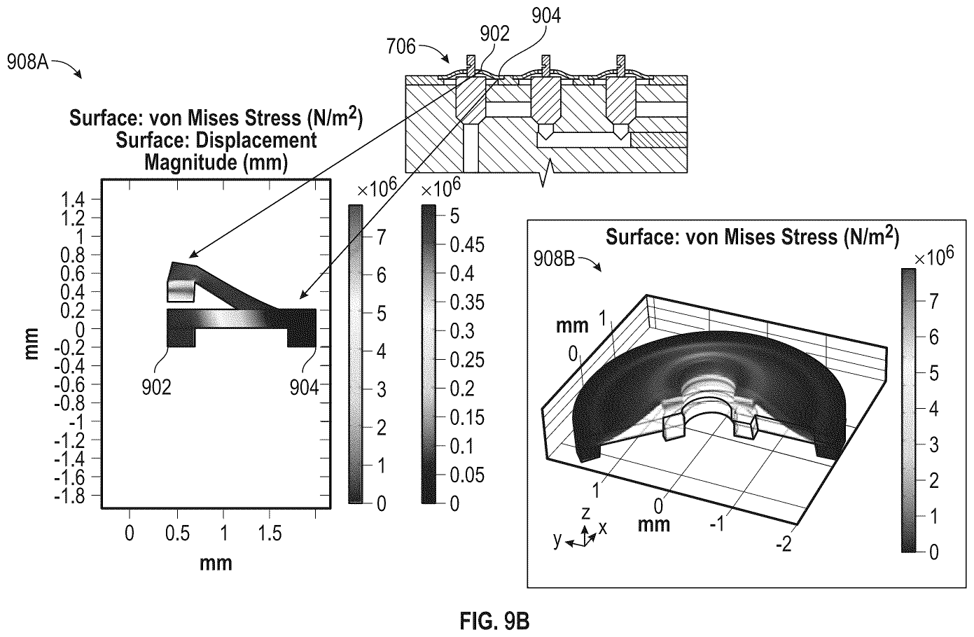

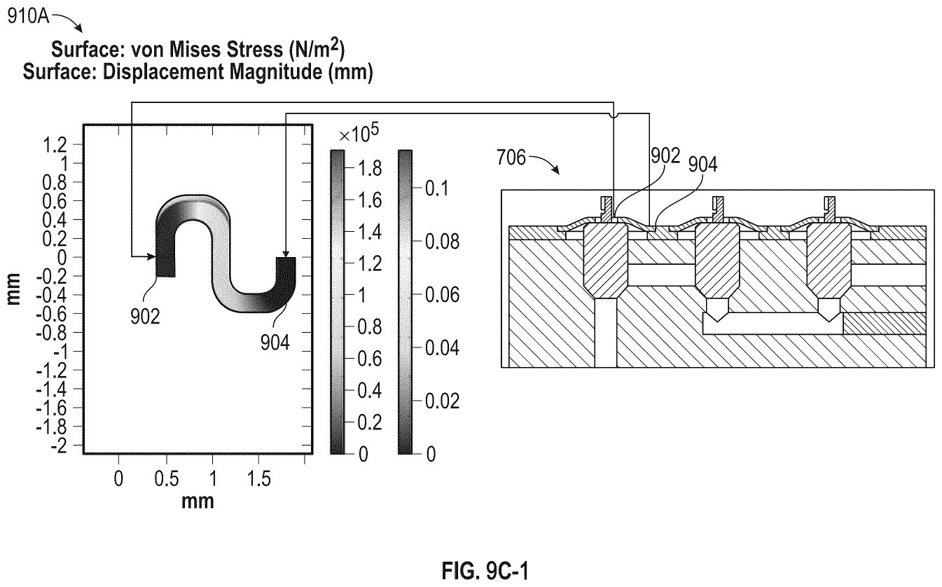

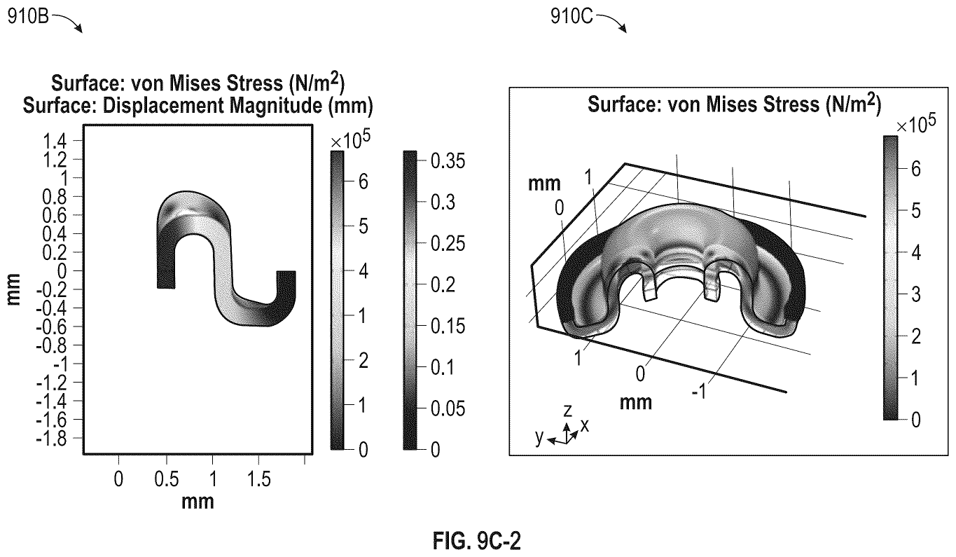

FIGS. 9A-9C-2 illustrate example disc spring shapes and simulations of displacement and force for various disc spring shapes illustrated. In the examples illustrated in FIGS. 9A and 9B, a disc spring comprises an approximately flat or frusto-conical shape. In some examples, the dimension of the disc spring may correspond to a dimension of a tube or diameter of a medication flow path. In some examples, the dimension of the disc spring may correspond to a geometry of a plunger. A force limit for exertion on a disc spring can be calculated based on a pressure limit multiplied by the open area of the disc spring. An open area can be calculated as: Open area=π(D outer/2)²−Dinner/2². A pressure limit can be: 300/760 atm or (4×10⁴)(N/m²).

In an example disc spring with an inner diameter of about 0.0 mm and an outer diameter is about 0.8 mm. A force limit may be calculated as 0.002 kgf. A safe force limit may be some percentage smaller than the total force limit. For example, in the same example, a safety force limit may be 10 gf.

FIG. 9A shows diagrams 906A, 906B illustrating example stress on the surface of a disc spring during displacement of about 0.2 mm of an interior point 902 of the disc spring with respect to an exterior point 904. As shown, stress is greater on the displaced portion of the disc spring and can reach up to (2×10⁶)(N/m²) or more for a displacement of approximately 0.25 mm.

FIG. 9B shows diagrams 908A, 908B illustrating example stress on the surface of a disc spring during displacement of about 0.5 mm of an interior point 902 of the disc spring with respect to an exterior point 904. As shown, stress is greater on the displaced portion of the disc spring and can reach up to (8×10⁶)(N/m²) or more for a displacement of 0.5 mm.

FIGS. 9C-1 and 9C-2 show diagrams 910A, 910B, 910C illustrating example stress on the surface of a disc spring having an S-shaped curve between an interior point 902 and exterior point 904. Diagram 910A illustrates stress during displacement of about 0.12 mm of an interior point 902 of the disc spring with respect to an exterior point 904. As shown, stress is greater on the displaced portion of the disc spring and can reach up to (2×10⁶) (N/m²) or more for a displacement of 0.2 mm. Diagram 910B illustrates stress during displacement of about 0.35 mm of an interior point 902 of the disc spring with respect to an exterior point 904. As shown, stress is greater on the displaced portion of the disc spring and can reach up to (7×10⁶)(N/m²) or more for a displacement of 0.35 mm.

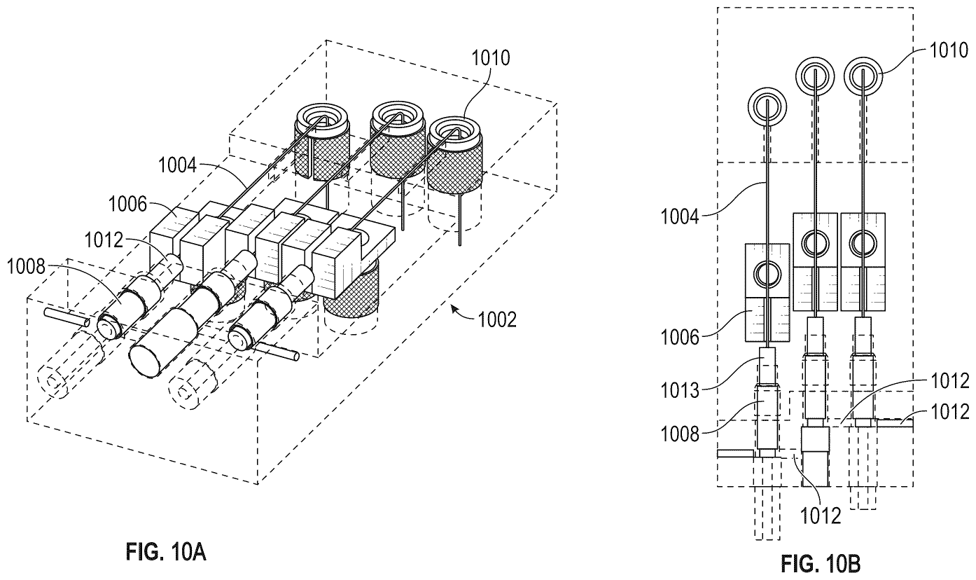

FIGS. 10A and 10B illustrates an example functioning of an example muscle wire pump 1002. In the illustrated example, a muscle wire pump 1002 may include a plurality of muscle wires or muscle springs 1004, one or more guides 1006 for the muscle wires 1004, one or more plungers (also referred to herein as plungers) 1008, and one or more flow paths 1012. More, fewer, and/or different components may also be used.

A pump may include a muscle wire 1004 for each plunger 1008. The muscle wire 1004 may include any material configured to contract when electrical current is applied. For example, the muscle wire 1004 may comprise a shape memory alloy (SMA). For example, the muscle wire 1004 may include a nitinol or a nickel titanium alloy. Other SMAs may also be used. In some examples, a muscle wire 1004 may be suspended so as to maintain tension. In some examples, a guide 1006 may be used to aid in support of the wire 1004. In some examples, a coupling component 1010 may be configured to hold a guide 1006, muscle wire 1004, or other components in place with respect to the pump or disease management system to which the pump is coupled. Additionally or alternatively, the coupling component 1010 may help guide or couple the muscle wire 1004 towards or to electronics of the pump configured to apply electrical current to the muscle wire. A muscle wire 1004 may be configured to connect to a plunger 1008. The plunger 1008 may be configured to block, directly or indirectly, a flow path 1012 of a medication. The assembly may include a plurality of plungers 1008 and muscle wires 1004. A spring 1013 (e.g., a disc spring) may be coupled to each plunger 1008 to provide force to the plunger 1008 in order to block the flow path.

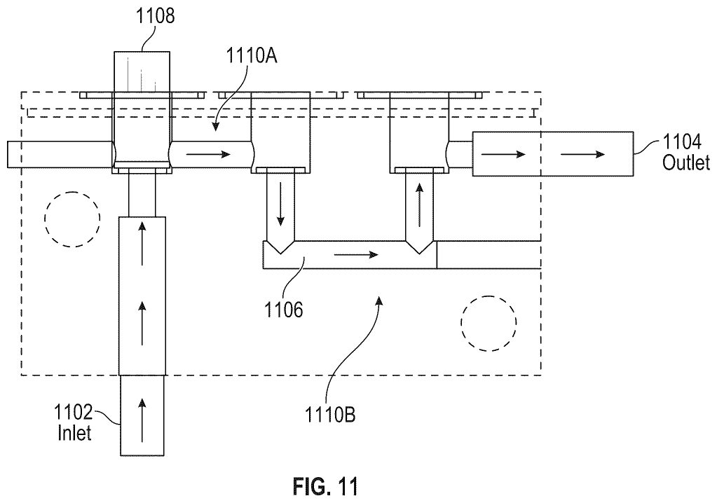

A controller may be configured to control the operation of the pump by operating the plurality of plungers in a sequence. FIG. 11 illustrates an example flow path 1106 from an inlet 1102, which may be associated with fluid from a medication pouch to an outlet 1104, which may be associated with a cannula configured to deliver medication to a patient. The fluid and/or medication pouch can be under pressure. In some examples, the pressure applied to the fluid pouch may be minimal, but can be sufficient to push the fluid into or through the flow path. As fluid leaves the fluid pouch, the pouch can collapse and continue to apply pressure to the fluid. Alternatively, the medication pouch does not need to be under pressure where the plungers are configured to generate negative pressure to pull fluid into the flow path. A plunger, such as a first plunger 1108, may be configured to be unactuated, closed, or blocking a flow path 1106 in a first configuration. In order to push medication through the inlet 1102 to the outlet 1104, the controller may open or lift the plunger 1108 so that medication flows in an area 1110A of the flow path between the first plunger and a second plunger. The first plunger and the second plunger may be lifted approximately simultaneously or at comparable times. The lifting of the first plunger and the second plunger at comparable times can create a vacuum that causes medication to flow into an empty cavity. This can allow medication to flow into the area 1110A. The medication can flow into the flow path either by positive pressure applied at the medication pouch or by negative pressure generated by the retraction of each plunger. The controller may close or lower the first plunger to close or block the flow path. Next, the second plunger may be closed or block the flow path at approximately the same time or comparable times as the third plunger is lifted. This can reduce the amount of fluid that may flow back from the patient due to the vacuum formed and also creates positive pressure on the medication in the flow path as the second plunger releases and presses down on the medication. This can also cause the medication to flow from the area 1110B between the second plunger towards the outlet 1104. In some examples, the closing of the second plunger can be slightly delayed with respect to the contraction of the third plunger. This delay can be accounted for in the timing such that the second plunger may be released before the third plunger is raised so that both plungers move simultaneously or nearly simultaneously. This may be due to the increase in temperature of a muscle wire connected to the second plunger. In some examples, the controller may open and close the first plunger and the second plunger approximately simultaneously and open the second plunger when the first plunger and the third plunger are closed. In performing this sequence, medication may be pumped from the inlet 1102 to the outlet 1104.

### Pump Actuator Feedback Mechanism

In some examples, a pump system can include a feedback control system. FIG. 12A -14 illustrate example aspects of feedback control for an example pump system. As illustrated in FIG. 12A-12C, a feedback mechanism may include, but is not limited to, a top feedback PCB 1206, a bottom feedback PCB 1210, one or more contact rings 1202 (mounted to the plunger 1218) and one or more spacers 1208 to mount PCBs. During assembly, the bottom feedback PCB 1210 may be mounted to the main housing 1220 first. Other components may then be assembled layer by layer on top of the bottom feedback PCB 1210. One or more components of the feedback system can be held in place and aligned using one or more components, such as alignment pins 1214 and one or more latches 1216. For example, a top PCB 1206 and bottom PCB 1210 may be aligned and held in place using the alignment pins 1214 and one or more latches 1216. A pump housing 1220 may be made with a plastic, such as a cyclic olefin copolymer (COC) or polypropylene (PP) material. It is also possible that the housing is made with other plastic polymer, including but not limited to high-density poly ethylene (HDPE) with SiO₂ coating. A contact ring 1202 may be made with a high conductive material. The contact ring 1202 can be coated with carbon filled polymer or electrically conductive silicone. A pattern and/or layout of the feedback PCB can significantly enhance the detection when the contact ring shorts the circuit. An example pattern 1212 is illustrated in FIG. 12B.

The pump may include feedback notification to a controller when the plungers have traveled a desired distance so as not to damage the shape memory alloy (such as a disc spring) by over stretching it. The feedback can also give better control of bolus delivery by tightly controlling the plunger travel distance. The feedback control signals may additionally or alternatively indicate when the plunger is fully seated and hence, blocking fluid flow. The feedback control signals may additionally or alternatively indicated when the plunger is fully open and hence, allowing fluid flow. A feedback control may occur based on a short created by the plunger between two traces on a printed circuit board 1206, 1210 that is located above or below the plunger. The printed circuit boards may include one or more contact rings 1202 configured to detect contact by the plunger. One or more spacers 1208 between the circuit boards may prevent accidental or unintended shorts. The controller reads the feedback control signal and when it detects a short circuit, the controller can disable the plunger from moving any further.

When a top feedback PCB 1206 detects that plunger 1218 moves to the top position, the plunger may be held in place. Pulse width modulation (PWM) can be activated, which maintains the temperature in the muscle wire. Accordingly, the muscle wire can stay stationary and hold the plunger stationary. Thus, the muscle wire may not pull with additional strain or relax in any strain. Additionally or alternatively, when the top feedback PCB 1206 detects that plunger 1218 moves to a top position, it will cut down the power for the muscle wire. When the top feedback detects the plunger is disengaged, it can power up again. This back-and-forth power cycle will allow the plunger to stay near the top position. When the bottom feedback PCB 1210 detects the plunger touches the bottom, the system knows that the plunger is fully closed. This can ensure the plunger is fully closed.

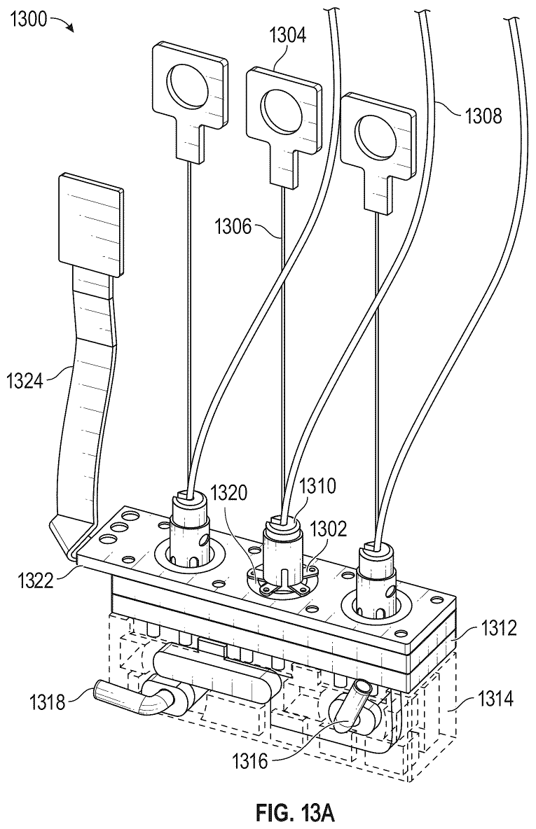

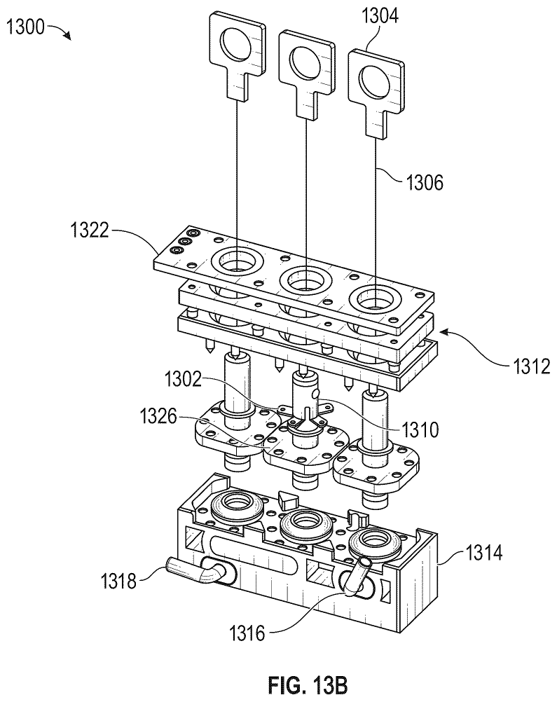

FIG. 13A illustrates an example of a muscle wire pump 1300 that includes a feedback mechanism 1302. FIG. 13B illustrates an exploded view of the muscle wire pump 1300. The muscle wire pump 1300 can include features as discussed with reference to FIGS. 4A-5L. The muscle wire pump 1300 can include a plurality of plungers 1310, a pump housing 1312, a pump assembly 1314, a plate assembly 1322, a pump outlet 1316, and a pump inlet 1318. Each plunger 1310 can include a feedback mechanism 1302. The feedback mechanism 1302 can be coupled to the plunger 1310 and translatably move with the plunger 1310 when the plunger 1310 is actuated by the muscle wire 1306. The plate assembly 1322 can include a feedback ring 1320 corresponding to each feedback mechanism 1302. The plunger 1310 can include a plate component 1326, similar to the plate component 2234 discussed with reference to FIGS. 5D and 5E. The feedback mechanism 1302 may work in combination with other components of the muscle wire pump 1300 to maximize control, accuracy, precision, and the like of the muscle wire pump 1300. For example, the muscle wire pump 1300 can include a plurality of connectors 1304, a plurality of muscle wires 1306, a plurality of plungers 1310, and a plurality of conductive wires 1308 configured to connect to the muscle wire 1306. The connectors 1304 can anchor a distal end of the muscle wire 1306, allowing the muscle wire 1306 to, when actuated, pull against the plunger 1310 to move the plunger 1310 in a direction away from the pump assembly 1314 towards the anchored plurality of connectors 1304. The plurality of connectors 1304 can be anchored against a portion of the pump device. It is to be understood that various shapes may be suitable for the 1304 so long as the plurality of connectors 1304 couple the muscle wire 1306 to the anchor site. In some examples, the plurality of connectors 1304 can be similar to the guide components 2232 discussed with reference to FIG. 5B, and can clip into an anchor site on an interior surface of the second portion 104, with reference to FIGS. 1A-1M. The plate component 1326, which may act as a disc spring, can oppose the pull of the muscle wire 1306 on the plunger 1310 and can return the plunger 1310 to an unactuated (relaxed) position after stimulation of the muscle wire 1306 by the conductive wire 1308 ceases.

The conductive wire 1308 may be used to promote or allow a signal to be sent to the muscle wires 1306 so as to actuate the muscle wire 1306. The conductive wire 1308 can be used to provide current to or heat the muscle wire 1306. The muscle wire pump 1300 can further include at least one feedback ring 1320 that may allow the feedback mechanism 1302 to monitor the position and status of each plunger 1310. The feedback ring 1320 can act as a stationary electro-mechanical contact. The feedback mechanism 1302 can act as a mobile electro-mechanical contact of the plunger 1310. In some examples, there can be a feedback ring 1320 connected to each plunger 1310 within the muscle wire pump. For example, the feedback ring 1320 can sense contact with the feedback mechanism 1302, allowing for monitoring or control of the status (e.g., actuated or relaxed) of each of the plungers 1310. The muscle wire pump 1300 can also include a cable 1324 (e.g., a flex cable) configured to connect to the feedback mechanism 1302. The cable 1324 can convey signals from the feedback mechanism 1302 and/or feedback ring 1320 to a controller of the muscle wire pump 1300.

In some examples, as shown in FIG. 13A, when the plunger is in an unactuated (e.g., closed or relaxed) position, the feedback ring 1320 can contact the feedback mechanism 1302, which may trigger a signal sent to a controller of the muscle wire pump 1300. The signal may allow the controller of the muscle wire pump 1300 to verify the plunger 1310 is in an unactuated position. In some examples, when the feedback ring 1320 is not in contact with the feedback mechanism 1302, the feedback mechanism 1302 may not send a signal due to lack of contact with the feedback ring 1320.

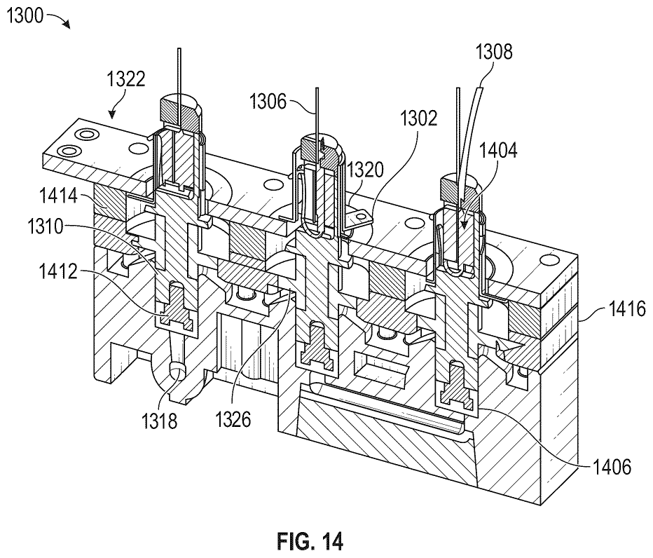

Though FIGS. 13A-14A show an example muscle wire pump 1300 where the feedback mechanism 1302 and feedback ring 1320 are in contact when the 1310 is an unactuated position, it is to be understood that other implementations are possible. In other examples, when the plunger 1310 is in an actuated position, the feedback ring can contact the feedback mechanism 1302, which may trigger a signal sent to the controller of the muscle wire pump 1300. The signal may allow the feedback mechanism to verify the plunger is in an actuated position.

In yet further examples, there may be two feedback rings per plunger 1310. In such examples, the first of the two feedback rings 1320 can contact the feedback mechanism 1302 when the plunger 1310 is in an unactuated position, and the second feedback ring can contact the feedback mechanism 1302 when the plunger 1310 is in the actuated position. Based on sensed contact of the feedback mechanism 1302 with either of the first or second feedback rings, a controller of the muscle wire pump 1300 can verify whether the 1310 is in an actuated position or an unactuated (relaxed) position.

FIG. 14 illustrates a cross-section view of FIG. 13A. FIG. 13A shows, at least in part, an example integration, connection, and/or coupling of components of the muscle wire pump 1300 with respect to the feedback mechanism. As shown in FIG. 14, the muscle wire pump 1300 can include one or more feedback rings 1320 of the plate assembly 1322 that can contact the feedback mechanism 1302 of one of the plungers 1310, the plate assembly 1322 (which can include a PCB), a crimp connector 1404, at least one conductive wire 1308, a seal 1406, at least one muscle wire 1306, an inlet 1318, an adapter 1412 configured to connect to the plunger 1310, a spacer 1414, and a lid 1416. In some examples, the spacer 1414 and lid 1416 may be integrated into a single component, and may form a housing assembly 224 as discussed with reference to FIGS. 4B-1 and 4B-2. The plate assembly 1322 can include the feedback ring 1320. When the plunger 1310 is in a particular position (e.g., actuated or unactuated), the feedback mechanism 1302 can directly contact the feedback ring 1320. The crimp connector 1404 may be configured to couple each conductive wire each muscle wire for each of the plurality of plungers within the muscle wire pump. Accordingly, the contact between the feedback mechanism 1302 and feedback ring 1320 can cause a signal that allows for the monitor or control of the plungers 1310.

As shown in FIG. 14, the conductive wire 1308 can exit an internal compartment of the plunger at an exit point at a distal end of the plunger 1310.

The feedback mechanism allows a controller to verify the position of the plungers. A feedback signal can be used to allow the controller to know whether the plunger is in an actuated or unactuated position. In some examples, a feedback signal can be used to allow the controller to know whether the plunger is partially or fully open. The controller may be able to calibrate stimulation of the muscle wire 1306 depending on signal from the feedback mechanism 1302 and feedback ring 1320.

For instance, in examples where the feedback ring 1320 and feedback mechanism 1302 contact when the plunger 1310 is fully actuated, the controller can determine the corresponding minimum current and/or temperature of the muscle wire 1306 needed to cause the feedback mechanism 1302 to contact the feedback ring 1320. The controller can then determine, based on this current and/or temperature value, corresponding stimulation needed to actuate the plunger 1310 to a fraction of the fully actuated position (e.g., 25, 50, 75, or 100% of the fully actuated position).

In examples where the feedback ring 1320 and the feedback mechanism 1302 contact when the plunger 1310 is not actuated, the controller can determine the corresponding minimum current and/or temperature of the muscle wire 1306 needed to cause the feedback mechanism 1302 to cease contacting the feedback ring 1320. The controller can then determine, based on this current and/or temperature value, corresponding stimulation needed to actuate the plunger 1310 to a fraction of the fully actuated position (e.g., 25, 50, 75, or 100% of the fully actuated position).

In some examples, the controller may be able to partially actuate the plunger 1310 to control the bolus amount or amount of fluid pumped through the pump system. In some examples, the level of actuation of the plunger can determine the rate at which the fluid flows through the pump system because the level of opening of the plunger can determine the volume of the fluid. Advantageously, this can save power in cases where at least some of the plungers do not need to be fully opened to deliver a sufficient bolus.

The controller of the muscle wire pump 1300 may specifically time contraction of each of the plungers 1310 so that fluid can move from one direction to the other, such as from a medication bladder or pouch towards a catheter or cannula embedded in a patient. The controller can use signal of the feedback ring 1320 and feedback mechanism 1302 to verify that accurate timing of plunger actuation is being implemented. In this manner, the amount of medication delivered to the patient can be controlled because only a small portion of the medication is released at any given time due to the offset timing of the plunger openings.

A controlled amount of medication delivered to the patient can be precise and accurate to the dosage of medication needed. The arrangement, timing or level of opening of the plungers can be finely tuned, allowing for a controlled and/or precise amount of medication to be delivered to the patient.

Advantageously, the feedback mechanism can also provide a safety factor to the muscle wire pump system by verifying plunger position and/or movement.

### Precise Control of Pump Actuators

A shape memory alloy (SMA) actuator may be used to control a position of a plunger of a microinfusion pump. A processor can induce deformation of the SMA actuator (also referred to herein as a muscle wire). An electrical property (e.g., resistance or voltage) of the SMA actuator can be measured to determine a displacement of the plunger. In some examples, an environmental sensor may be used to tune control of the SMA actuator to ensure precise actuation as environmental conditions of the microinfusion pump change. Advantageously, such control can allow for a range of target plunger displacements, in addition to or alternatively to a binary choice of fully open (i.e., maximum displacement of the plunger) or fully closed (i.e., minimal or no displacement of the plunger) with respect to plunger displacement. In some examples and in accordance with the present disclosure, two or more of the SMA actuators may be positioned in series along a flow path.

FIGS. 15A-15I are schematic diagrams of an illustrative example pumps 1500, 1530, and 1550, for example a micro-infusion pumps, and volume dispense control features. FIGS. 15A-15C show an example pump 1500. FIGS. 15A, 15D, and 15G show the SMA actuator in an unactuated plunger position. FIGS. 15B, 15E, and 15H show the SMA actuator in an open plunger position. FIGS. 15C, 15F, and 15I show the SMA actuator in a partially actuated position. It is to be understood that, as FIGS. 15A-15I are schematic drawings, certain aspects of the pump 1500 have been simplified. Greater structural detail of such pumps are discussed with reference to FIGS. 4A-14. Though FIGS. 15A-15I depict a single plunger 1504 for sake of simplicity, it is to be understood that a pump in accordance with the present disclosure can include more than one plunger, for example three plungers, in series along a fluid medication flow path. Various features of FIGS. 15A-15C can be combined, to the extent they are compatible.

With reference to FIGS. 15A-15C, the pump 1500 may include a muscle wire 1502, a plunger 1504, an anchor 1506, a mobile plunger electro-mechanical contact 1508, a stationary electro-mechanical contact 1510, an inlet 1512, an outlet 1514, a cylinder 1516, a first electrical wire 1518, a second electrical wire 1520, and a third electrical wire 1524. Any or all of the first electrical wire 1518, second electrical wire 1520, or third electrical wire 1524 may be in communication with electrical sensors and/or power sources, which are in turn in communication with a processor. The processor may be capable of receiving voltage, current, and/or resistance of the muscle wire 1502 (which may be or include a SMA wire), such as may be measured by the electric sensor(s) or otherwise calculated or estimated. Similarly numbered elements in FIG. 15D-15F refer to the same components as discussed with reference to FIG. 15A-15C.

The muscle wire 1502 extends between the plunger 1504 and the anchor 1506. The anchor 1506 may be similar to or include the connector 1304, with reference to FIG. 13A. The plunger 1504 is positioned within the cylinder 1516. Generally, the amplitude of displacement of the cylinder 1516 from an unactuated position (e.g., as shown in FIG. 15A, also referred to herein as a “relaxed” position or a “closed” position) to an actuated position (e.g., as shown in FIG. 15B, also referred to herein as an “open” position) controls the volume dispensed by the pump. For instance, an intermediate displacement (e.g., as shown in FIG. 15C) may result in a smaller volume dispensed than for a full displacement (e.g., as shown in FIG. 15B).

In operation of the pumps 1500, 1530, and 1550, the processor can cause an electric current to be applied to the muscle wire 1502. In some examples, the electric current can be modulated with Pulse Width Modulation (PWM). Joule's heating activates the SMA material of the muscle wire 1502, causing the muscle wire 1502 to contract and pull the plunger 1504 from unactuated position to the actuated position. Termination of the current across the muscle wire 1502 can allow the muscle wire 1502 to cool down and relax to the initial length causing the plunger 1504 to move back to the unactuated position.

Though FIGS. 15A-15C depict a pump 1500 capable of sensing the plunger 1504 being in an actuated position (e.g., by contact of the stationary electro-mechanical contact to the 1508 while in the actuated position), it is to be understood that, in other arrangements, a pump may be capable of sensing when a plunger is in unactuated state via contact of two or more sensing components. FIGS. 15D-15F show an example pump 1530 that can sense the plunger 1504 being in an unactuated position. For instance, in FIG. 15D, the stationary electro-mechanical contact 1510 and mobile plunger electro-mechanical contact 1508 are in contact. However, when the plunger 1504 is in the fully actuated position (as shown in FIG. 15E) or a partially actuated position (as shown in FIG. 15F), the stationary electro-mechanical contact 1510 and mobile plunger electro-mechanical contact 1508 are not in contact.

In some examples, the pump may include a spring (e.g., a disc spring in accordance with the present disclosure) that can oppose contraction of the muscle wire 1502. FIGS. 15G-15I show an example pump 1550 that includes a disc spring 1526. In FIG. 15G, the plunger 1504 is in an unactuated state and the disc spring 1526 is relaxed (e.g., not stretched). When the muscle wire 1502 is not contracted, the disc spring 1526 can hold the plunger 1504 in the unactuated position. In FIG. 15H, the plunger 1504 is fully actuated, and the disc spring 1526 is maximally stretched. In the fully actuated position, the disc spring 1526 pulls against the contraction of the muscle wire 1502 with maximal force. In FIG. 15I, the plunger 1504 is partially actuated, and the disc spring 1526 is stretched an intermediate distance. In an partially actuated position, the disc spring 1526 pulls against the contraction of the muscle wire 1502 with intermediate force.

As shown in FIGS. 15G-15I, the plunger 1504 need not create a seal against an interior surface of the cylinder 1516. Instead, the disc spring 1526, coupled to edges of the cylinder 1516 and the plunger 1504, may provide a seal. Additionally, the plunger 1504 may only partially occupy the interior volume 1522 of the cylinder 1516, even when in an unactuated position. The pump 1550 can exert negative pressure on the inlet 1512 by actuating the plunger 1504 and moving (e.g., stretching or deforming) the disc spring 1526. The plunger 1504 need not block the flow path between the inlet 1512 and the outlet 1514 to cause movement of liquid into the interior volume 1522 from the inlet 1512.

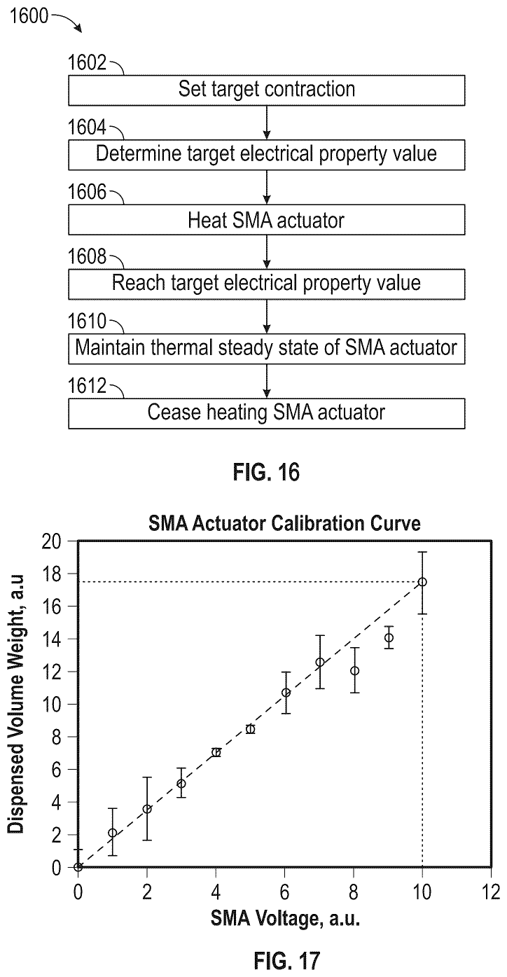

In some examples, the processor can use a voltage measured across the muscle wire 1502 to control and adjust contraction of the muscle wire 1502, thereby controlling and adjusting the corresponding displacement of the plunger 1504 and volume dispensed from the pump 1500. FIG. 16 diagrams an example method 1600 of controlling the SMA wire. The method 1600 will be discussed with reference to FIG. 16 and the elements of FIGS. 15A-15C. It is to be understood that similar methods can be implemented with the pumps 1530, 1550 of FIGS. 15D-15I.

At step 1602, the controller can set a target contraction of the SMA wire. The processor can set a target contraction based at least in part on a desired volume to dispense from the pump. The processor can set a target contraction based at least in part on the contraction required to contact the mobile plunger electro-mechanical contact 1508 to the stationary electro-mechanical contact 1510. The stationary electro-mechanical contact 1510 does not move with the plunger 1504.

At step 1604, the controller can determine a target electrical property of the muscle wire 1502. The processor can set a target electrical property value (e.g., voltage or resistance) corresponding to the desired contraction of the muscle wire 1502, which itself may directly or indirectly correspond to displacement of the plunger 1504 and/or volume dispensed by the pump 1500 at the beginning of a pumping cycle.

At step 1606, the controller can cause an electric current to heat the muscle wire 1502. The SMA wire may be heated by applying a “high” duty cycle PWM to the muscle wire 1502. The pumping cycle starts from an unactuated plunger position where the plunger 1504 blocks the flow of liquid through the pump 1500, as shown in FIG. 15A. An electric current with a “high” duty cycle PWM can be applied to the muscle wire 1502 causing it to heat and contract.

At step 1608, the muscle wire 1502 reaches a target electrical property value. When contracting, the muscle wire 1502 pulls the plunger 1504 into an actuated position as shown in FIGS. 15B or 15C, which causes liquid to travel through the inlet 1512 to the interior volume 1522 of the cylinder 1516. The muscle wire 1502 can pull the plunger 1504 to a fully actuated position, as depicted in FIG. 15B, or to a partially actuated position, as depicted in FIG. 15C, depending on how much the muscle wire 1502 is heated. The extent of contraction of the muscle wire 1502 can be determined based on the measured electrical property value (e.g., voltage or resistance) of the muscle wire 1502. The processor can determine when the measured electrical property value of the muscle wire 1502 reaches the target.

At step 1610, the controller can maintain thermal steady state of the SMA actuator. In some examples, the SMA actuator can be maintained at a thermal stead state by switching to apply a “low” duty cycle PWM to the muscle wire 1502. When the electrical property value (e.g., voltage or resistance) measured across the muscle wire 1502 reaches the target value, the processor can switch the PWM duty cycle to a “low” value, thereby restricting the heat generated in the muscle wire 1502. In some examples, the “low” PWM duty cycle causes the muscle wire 1502 to generate heat equal to the heat lost from the muscle wire 1502 to the environment via conduction, convection, and radiation. In other words, the “low” PWM duty cycle may put the muscle wire 1502 in a thermal steady state. In such examples, the muscle wire 1502 may stop contracting and the plunger 1504 may remain at the desired displacement in an actuated position, allowing flow of liquid through the cylinder 1516 from the inlet 1512 to the outlet 1514. The processor may allow the plunger 1504 to remain at the desired position to allow for sufficient dispensation of liquid volume.

At step, 1612, the controller ceases heating the SMA actuator. In some examples, step 1612 can be accomplished by ceasing application of the PWM duty cycle. When the processor turns the “low” PWM duty cycle off, the muscle wire 1502 can cool and relax, allowing the plunger 1504 to return to the unactuated position (as shown in FIG. 15A) pushing the liquid out of the cylinder 1516 through the outlet 1514, blocking further flow through the inlet 1512, and ending the pumping cycle.

In some examples, the voltage measured across the muscle wire 1502 may be used by the processor for feedback control of the volume dispensed by the pump 1500. In some examples, the processor can compare voltage measured at the end of the “high” duty cycle PWM phase with the set target voltage. If the measured voltage is higher or lower than the set target voltage, the processor can adjust the “high” PWM duty cycle to speed up or slow down the heating of the muscle wire 1502 and increase or decrease the displacement of the plunger 1504 and dispensed volume, respectively. The processor can adjust the PWM duty cycle to an appropriate value in any suitable fashion, including but not limited to linear, binary, hashing or in accordance with a search algorithm.

In some examples, the processor may use the value of voltage or resistance measured across the muscle wire 1502 to stabilize the position of the plunger 1504 during the “low” duty cycle PWM phase. In some examples, the processor can compare voltage or resistance measured during the “low” PWM application with the set target voltage. If the measured voltage is higher or lower than the set target voltage, the processor can adjust the “low” PWM duty cycle to balance the heat generated in the muscle wire 1502 to be equal to heat lost to the environment by the muscle wire 1502 via conduction, convection, and radiation, thereby keeping the position of the plunger 1504 stable. The processor can adjust the PWM duty cycle to an appropriate value in any suitable fashion, including but not limited to linear, binary, hashing or in accordance with a search algorithm.

In some examples, variations in PWM amplitude, PWM duty cycle and duration of the electric current application can be used jointly or separately to control motion of the plunger 1504. In some examples, electric resistance of the muscle wire 1502 can be measured directly and used to control operation of the pump 1500.

In some examples, a processor may be capable of calibrating the volume dispensed by the pump 1500. Calibration may be performed by correlating the position of the plunger 1504 moved by the muscle wire 1502 with an electrical measurement (e.g., resistance and/or associated voltage) of the muscle wire 1502. In some examples, the position of the plunger 1504 is monitored using the mobile plunger electro-mechanical contact 1508 and the stationary electro-mechanical contact 1510. The mobile plunger electro-mechanical contact 1508 may be similar to—or the same as—the feedback ring 1320 discussed with reference to FIGS. 13A-14. The stationary electro-mechanical contact 1510 may be a part of a PCB (e.g., as discussed with reference to a PCB of plate assembly 1322 of FIGS. 13A-14). When electrical contact is made between mobile plunger electro-mechanical contact 1508 and the stationary electro-mechanical contact 1510, a circuit is formed and the processor can sense that the plunger 1504 is in the actuated position. In some examples, the processor can monitor the position of the plunger 1504 using capacitive measurements of the muscle wire 1502. In some examples, the processor can monitor the position of the plunger 1504 using inductive measurements of the muscle wire 1502. In some examples, the processor can monitor the position of the plunger 1504 using resistive measurements of the muscle wire 1502. In some examples, the processor can monitor the position of the plunger 1504 using magneto resistive measurements of the muscle wire 1502. In some examples, the processor can monitor the position of the plunger 1504 using optical measurements of the muscle wire 1502. In some examples, the processor can monitor the position of the plunger 1504 using Hall effect measurements of the muscle wire 1502. The electric resistance or associated voltage of muscle wire 1502 can be monitored by the controller via an electric wire in communication with the muscle wire 1502, for example the third electrical wire 1524.

In some examples, calibration of the volume dispensed by the pump 1500 is performed by applying a voltage and/or current to cause the muscle wire 1502 to contract sufficiently to the plunger 1504, so the mobile plunger electro-mechanical contact 1508 is brought into contact with the stationary electro-mechanical contact 1510, as shown in FIG. 15B. When in contact, the mobile plunger electro-mechanical contact 1508 and stationary electro-mechanical contact 1510 form a circuit with the second electrical wire 1520 and the outlet 1514. This displacement of the muscle wire 1502 shown in FIG. 15B is the maximal displacement. The volume dispensed by the pump 1500 at this maximal displacement of FIG. 15B may be the maximal dispensed volume. Electric resistance or associated voltage of the muscle wire 1502 corresponding to this maximal displacement/dispensed volume can be measured, for example via third electrical wire 1524. The volume dispensed by the pump 1500 at lower current or power can be measured together with corresponding resistance/voltage of the muscle wire 1502 and a pump calibration algorithm can be produced. Such a calibration algorithm can be used by the processor to set and adjust volume dispensed by the pump 1500 according to the treatment protocol.

FIG. 17 shows an example calibration curve for a SMA actuator in accordance with the present disclosure. In the calibration curve of FIG. 17, the volume dispensed by the pump is correlated to the voltage applied to the SMA wire. Maximum dispensed volume (i.e., a dispensed volume weight of about 17.5 arbitrary units (“a.u.”)), and corresponding SMA voltage (e.g., a voltage of 10 a.u.) are indicated. Though the calibration curve of FIG. 17 is depicted as linear, it is to be understood that any suitable function may be implemented to approximate the relationship between power applied to the SMA wire and dispense volume.

In some examples, the processor may additionally receive input from one or more environmental sensors (e.g., with reference to FIG. 1A, the environmental sensor 1148). The one or more environmental sensors may be able to sense environmental conditions that may affect the functioning of the muscle wire 1502. For example, the one or more environmental sensors may be capable of sensing ambient temperature, atmospheric pressure, humidity, or any other suitable environmental condition. In such examples, the processor may be capable of calibrating the volume dispensed by the pump 1500 based at least in part on measurements of the one or more environmental sensors. In such examples, pump calibration curves or functions may be obtained at different environmental conditions (e.g., ambient temperature, atmospheric pressure, humidity, etc.). The processor can access and use this information, such as in a pre-stored calibration algorithm or look-up table, to adjust operation (e.g., adjust a target displacement) of the pump 1500 in response to changes in environmental conditions when the pump 1500 is in use. As an illustrative example, the processor can set the pump 1500 to dispense a given volume. If a temperature sensor detects an ambient temperature change, the processor can select another value of target voltage or resistance for the muscle wire 1502 that corresponds to the set plunger 1504 displacement and pump dispense volume at the new temperature. To ensure consistent pump dispense volume, the controller can change voltage or current applied to the muscle wire 1502 to achieve the resistance or voltage associated with the target displacement at the new temperature.

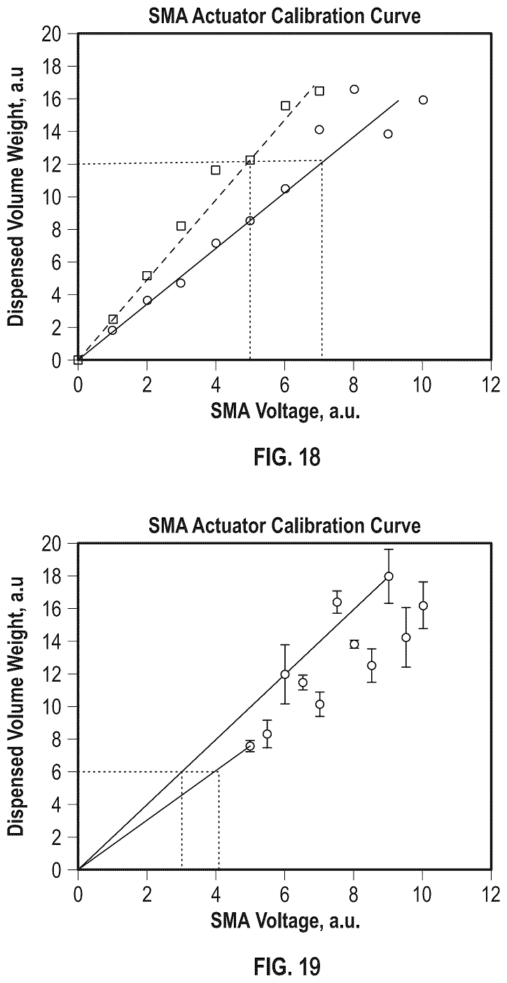

FIG. 18 includes micro infusion pump calibration curves for two different environmental conditions, a first ambient temperature and a second ambient temperature. To produce the same dispensed volume (i.e., 12 a.u.), the voltage applied to the SMA actuator must be changed to account for the difference in ambient temperature. For instance, for the first ambient temperature, the voltage applied may be about 5 a.u. to ensure a dispense volume of 12 a.u., whereas, for the second ambient temperature, the voltage applied may be about 7 a.u. to ensure a dispensed volume weight of about 12 a.u.

In conditions of mass production, each individual pump may have unique maximal dispensed volume, for example due at least in part to variation in material properties of its components and dimensional tolerances of its components and assembly. In accordance with the present disclosure, the maximal displacement for SMA actuators in each individual pump can be characterized. Based on the characterization, processors can select individual target resistances or target voltages that correspond to SMA displacement producing same standard dispense volume. FIG. 19 plots two example SMA actuator calibration curves, each curve corresponding to a different pump. Dotted lines indicate measured SMA voltages for each of the pumps (i.e., about 4.2 a.u. and 3 a.u. respectively) that yield the same or similar given dispense volume (i.e., 6 a.u.).

In some examples, in conditions of mass production, the present disclosure provides for segregating manufactured pumps into subgroups according to maximal dispense volume. For example, pumps capable of high maximal dispense volume can be selected for applications where delivery of high drug dosage is desirable, for example in pumps intended for use by adults. Pumps with low maximal dispensed volume can be selected for applications where delivery of low drug dosage is desirable, for example in pumps intended for pediatric use.

### Medication Delivery Flow

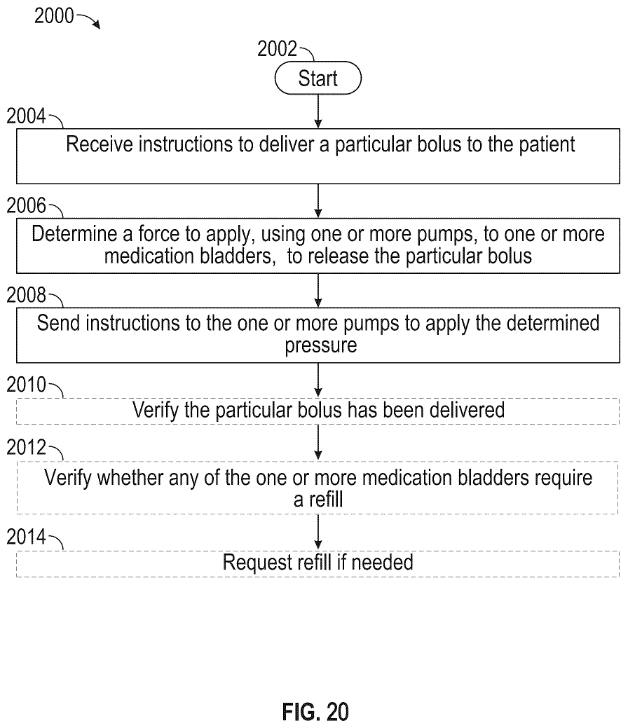

FIG. 20 illustrates an example routine 2000, which may be performed by a disease management system. The routine 2000 may be used to deliver fluid to a patient.

The routine 2000 begins in block 2002. The routine 2000 may begin in response to an event such as the receipt by the disease management system of instructions from a user or a patient. In some examples, instructions may be entered into a user device (e.g., smart phone, smart watch, etc.). Additionally, or alternatively, instructions may be received from one or more controllers within the disease management system, as described in more detail above. When the routine begins at block 2002, a medication bladder, such as the medication bladder 230, may be in a first fill state. The medication bladder in the first fill state may hold a first amount of fluid including chemical substances (e.g., medication, additives, or the like).

When the routine 2000 is initiated, a set of executable program instructions stored on one or more non-transitory computer-readable media (e.g., hard drive, flash memory, removable media, etc.) may be loaded into memory (e.g., random access memory or RAM) of a computing system, such as the disease management system shown in FIGS. 1A or 1B, and executed by one or more processors. In some examples, the routine 2000 or portions thereof may be implemented on multiple processors, serially or in parallel.

At block 2004, the disease management system can receive instructions to deliver a particular bolus of fluid to the patient. In some examples, the particular bolus could be calculated by the controller, as described in more detail above. In further examples, the particular bolus may be calculated based in part on data from sensors applied to the patient. Historical sensor data may also be used in calculating the particular bolus. Additionally, or alternatively, this historical data may be stored in physical storage devices, such as solid-state memory chips and/or magnetic disks, into a different state. Additionally, or alternatively, this historical data may be stored on one or more remote storage devices. In some examples, the controller can verify whether the medication bladder holds an amount of fluid including chemical substances that is greater than the particular bolus. In further examples, the controller can verify whether the medication bladder in the first fill state holds a first amount of fluid that is greater than the particular bolus. Additionally, or alternatively, the controller may also verify whether the medication bladder requires a refill. For example, the controller may check the amount of medication against a threshold. The threshold may be predefined and/or set by the user. This threshold may be an approximate percentage of a maximum volume of a medication bladder, such as the medication bladder 230. For example, the threshold may be approximately 5% of the maximum volume of the medication bladder. In other examples, the threshold may be approximately 0% of the maximum volume of the medication bladder.

At block 2006, the disease management system can determine a pressure to apply, using one or more pumps, to one or more medication bladders to release the particular bolus of fluid. In some examples, the pressure could be calculated by the controller. In further examples, the pressure may be calculated based in part on data from sensors applied to the patient. Historical sensor data may also be used in calculating the pressure. Additionally, or alternatively, this historical data may be stored in physical storage devices, such as solid state memory chips and/or magnetic disks, into a different state. Additionally, or alternatively, this historical data may be stored on one or more remote storage devices. The disease management system may incorporate aspects of method 1600, discussed with reference to FIG. 16. Environmental sensor data may be used as discussed herein to determine whether electrical signals sent to the pump need to be adjusted in order to account for environmental conditions, as discussed herein.

At block 2008, the disease management system may send instructions to the one or more pumps to apply the pressure, as described with respect to FIGS. 1A-1B. Additionally, or alternatively, the disease management system may send instructions to one or more pressure applicators, as described with respect to FIG. 1B. In some examples, instructions may be sent from one or more controllers within the disease management system, as described in more detail above. With reference to the illustrative examples of FIGS. 3A-3C, in response to this external pressure the medication bladder 230 is configured to cause an amount of fluid, based on the particular bolus, to leave an outlet of the medication bladder 230, such as the output port 2321.

In some examples, as described above, the medication bladder 230 may be configured such that at least part of the first portion 231 moves towards the second portion 232 in response to the pressure. The first portion 231 may apply a pressure against the fluid and/or against the second portion 232, which corresponds with the pressure applied by the pump.

After the amount of fluid leaves the medication bladder 230, the medication bladder may be in a second fill state. The second fill state may comprise a second amount of fluid. The second amount of fluid may be less than the first amount of fluid by approximately the amount of the particular bolus.

At block 2010, the disease management system may optionally verify whether the particular bolus has been delivered. For example, in some examples, the controller may access sensor data related to the one or more medication bladder (e.g., weight) to determine whether the particular bolus has been delivered. In some examples, this may comprise the medication bladder in the first fill state to the medication bladder in the second fill state. For example, the second amount of fluid may be compared to the first amount of fluid to determine whether the particular bolus has been delivered. In other examples, the controller may access sensor data relating to the patient to determine whether the fluid has taken effect as expected. As discussed, the fluid may include medication such as insulin, and the sensor data may indicate whether the insulin has been delivered. For example, the sensor data may include blood glucose level which the controller may compare to a prior blood glucose level to determine whether the insulin has had the desired effect. In further examples, the controller may use information on whether the fluid has taken effect as expected to determine whether to send instructions to provide an additional bolus of fluid to the patient. The amount of the particular bolus may be calculated by the controller, as described with respect to FIG. 1A. In some examples, the additional bolus may be requested regardless of whether the delivery of the particular bolus has been verified. In some examples, verification of whether the particular bolus has been delivered, may not occur. In some examples, the pump need not verify that the particular bolus has been delivered. The pump may be sufficiently precise and consistent that no verification of bolus delivery is needed.

At block 2012, the disease management system may optionally verify whether any of the one or more medication bladders require refilling. As discussed above, in some examples, the controller may check the amount of medication against a threshold. For example, the second amount of medication of the medication in the second fill state may be checked against the threshold to determine whether a refill is required. The threshold may be preset or set by the user. This threshold may be an approximate percentage of a maximum volume of a medication bladder, such as the medication bladder 230. For example, the threshold may be approximately 5% of the maximum volume of the medication bladder. In other examples, the threshold may be approximately 0% of the maximum volume of the medication bladder. In some examples, the disease management system may track the amount of medication remaining in the one or more medication bladder by subtracting past-administered bolus amounts from an initial medication amount that was present in the medication bladder before use by the disease management system. In such examples, the disease management system may not include sensors to measure properties of the one or more medication bladder.

Additionally, or alternatively, the controller may access sensor data related to the one or more medication bladders (e.g., weight) to determine whether the one or more medication bladders require refill. In some examples, verification of whether the one or more medication bladders require refill, may not occur.

At block 2014, the disease management system may optionally request a refill of any of the one or more medication bladders. For example, in some examples, after determining whether any of the one or more medication bladders requires a refill, the disease management system may request a refill of any of the one or more medication bladders that were determined to require refill. Additionally, or alternatively, the disease management system can request a refill when any of the one or more medication bladders is determined to hold a lesser amount than a particular amount of fluid specified by the system and/or by a user. Additionally, or alternatively, the disease management system can request a refill of any of the one or more medication bladders after a set period of time has passed. The time period may be related to an expiration date of the fluid. In some examples, the period of time may be set by the controller of the disease management system. Additionally, or alternatively, the period of time may be set by a user (e.g., by entry into a user device, such as a smart phone or smart watch). However, it is also possible that the disease management system may not request refills.

### Process for Release of Medication From a Medication Bladder

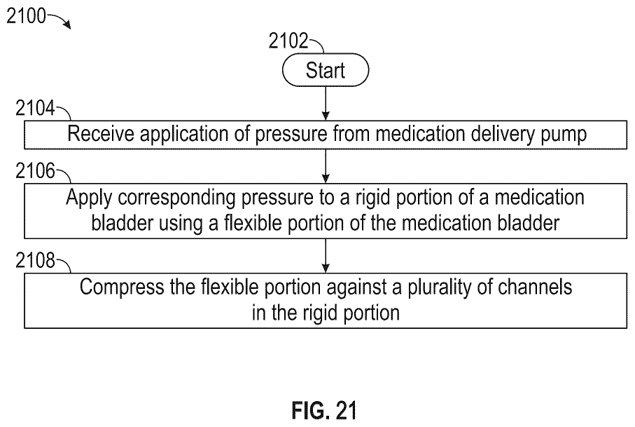

FIG. 21 illustrates an example process 2100 for release of medication from a medication bladder. The medication bladder may be similar to the medication bladder 230 shown in FIGS. 3A-3C, as described in more detail above. The process begins at block 2102. The process may begin in response to an event, such as configuration of the medication bladder for use in a disease management system, as described in more detail above.

At block 2104, the medication bladder may receive an application of pressure from a medication delivery pump (e.g., pump 1130 of FIG. 1A, pump 1186 of FIG. 1B, etc.). The medication delivery pump may be capable of exerting negative pressure on the fluid within the medication bladder. Prior to application of pressure by the medication delivery pump, internal pressure provided by the surfaces and structures of the medication bladder, as described above with respect to FIGS. 3A-3C, may be applied to bring the medication into communication with the medication delivery pump. Additionally, or alternatively, the medication delivery pump may apply a small pressure to bring fluid including chemical substances (e.g., medications, additives, and the like) in communication with the medication delivery pump. Once in communication with the fluid, the medication delivery pump may then apply pressure to deliver a specified bolus. The applied pressure may be in accordance with instructions from a controller, such as controller 1138 of FIG. 1A. The controller may provide instructions to achieve delivery a specified bolus to a specified destination, such as a patient or another medication bladder.

At block 2106, the pressure applied by the medication delivery pump may be converted into a pressure applied by a flexible portion of the fluid to a rigid portion of a medication bladder. The flexible portion may comprise a soft film, as described in more detail above. With reference to FIGS. 3A-3C, in some examples, the applied pressure may cause the first portion 231 to begin to collapse towards the second portion 232.

At block 2108, the amount of fluid in the medication bladder may be at a level that requires compression of the flexible portion against a plurality of channels in the rigid portion to continue release of fluid from the medication bladder. With continued reference to FIGS. 3A-3C, If the flexible portion is flush with the bulk of the second portion 232, the applied pressure may need to cause the first portion 231 to compress against the channels 2324 of FIG. 3C.

At block 2108, the flexible portion may compress against the plurality of channels in the rigid portion, as described in more detail above, to continue release of fluid. With continued reference to FIGS. 3A-3C, the compression may cause the fluid remaining in the channel to flow towards the outlet port 2321. The flow of fluid including chemical substances from the medication bladder may be further be facilitated by anti-aggregation coatings on the interior surfaces of the first portion 231 and/or the second portion 232.

### Injection System

The device 100 can include an injection system for introducing a cannula to the user. Once the cannula is inserted, the pump system 220 can begin providing fluid medication to the user. With reference to an illustrative example, FIGS. 22A-22B show a back perspective view 210a and a front perspective view 210b of an injection system 210 (which may also be referred to herein as “cannula insertion device” and/or “insertion device”), respectively. In some examples, the pump system 220 may be in communication with the injection system 210. As disclosed in further detail herein, the injection system 210 may be part of a disease management system as described herein. For example, the injection system 210 may be used as a needle insertion device. As described herein, reducing the overall profile of a medication injection system can improve patient comfort and maneuverability. In this way, the injection system 210 may provide improved insertion of a cannula or other device into a patient, while maintaining a low profile. In some examples, the injection system 210 may be used to insert a cannula or other device into an insertion site (also referred to as a “tissue site”) on the skin of a patient. In some implementations, the injection system 210 may insert the cannula at an angle at the insertion site on a patient. For example, the injection system 210 may insert the cannula into a patient at approximately a 45-degree angle, at an angle less than a 45-degrees, or at an angle above 45-degrees, with respect to a plane of the tissue site (and/or with respect to a plane of the injection system 210).

FIGS. 22C-22H illustrate various views of the injection system 210. FIG. 22C shows the injection system 210 from a first view 210c. FIG. 22D shows the injection system 210 from a second view 210d. FIG. 22E shows the injection system 210 from a third view 210e. FIG. 22F shows the injection system 210 from a fourth view 210f. FIG. 22G shows the injection system 210 from a fifth view 210g. FIG. 22H shows the injection system 210 from a sixth view 210h. FIG. 22I shows an exploded view of the injection system 210. The injection system 210 may include a trigger release 211 including a muscle wire 218, an enclosure 212, a trigger support component 213, a conduit 214 in fluid communication with a cannula 227, a needle assembly 215 including a needle 217, and a cannula carrier 216.

FIGS. 22J-22K illustrate a first view and a second view of the trigger release 211. The trigger release 211 may include a first guide component 2111, a first wire 218, a trigger pin 2113, a second guide component 2114, and a second wire 2115. The first guide component 2111 and the second guide component 2114 may be similar to the guide components 2232 discussed with reference to FIGS. 5A and 5B. The first guide component 2111 may couple to a first end of the muscle wire 218. The second guide component 2114 may couple to a second end of the muscle wire 218. In some examples, the muscle wire 218 can be secured within the first guide component 2111 and/or the second guide component 2114 with an adhesive. In some examples, the first guide component 2111 and/or the second guide component 2114 do not act as a crimp. The first guide component 2111 and the muscle wire 218 may couple to other components (such as an anchor 136 of a PCB or other anchor attached to another interior component of the device 100). The second guide component 2114 can be positioned within a slot of the trigger pin 2113. The first guide component 2111 can be positioned within a receiving slot of an anchor component. The anchor component, may, in turn, be attached to a static component of the device 100. The muscle wire 218 may include materials, such as SMA, as disclosed herein.

As illustrated in FIG. 22L, the trigger release 211 (via the trigger pin 2113) may me positioned within the enclosure 212 so as to prevent movement of the cannula carrier 216. The trigger release 211, once actuated, may trigger injection system 210. For example, the trigger release 211 may cause the trigger to activate (or deactivate), inserting the needle 217 into the patient. The trigger release 211 may be controlled by a controller of the device 100 via the second wire 2115. When the muscle wire 218 is actuated (e.g., by stimulation via second wire 2115), the first wire 218 can cause retraction of the trigger pin 2113, which in turn can cause activation of the trigger release 211. For example, the trigger release 211 may adjust a position in response to motion of one or more plunger. In this way, in response to motion of the plunger, the trigger release 211 may adjust a position to activate the needle assembly 215, causing the needle assembly 215 to extend from the enclosure 212 to a tissue site of a patient. For example, when the trigger activates, springs within the enclosure 212 may actuate (for example, decompress), adjusting a position of the needle assembly 215, extending at least a portion of the needle assembly 215 from the enclosure 212. In this way, the needle assembly 215 may deliver medication to a patient.

FIGS. 22M-22N illustrate a first bottom view and a second bottom view of the needle assembly 215 and the cannula carrier 216. The cannula carrier 216 including a structural component 2161 configured to provide support for each of a first support member 2162a, 2162b, and 2162c. The support members 2162a,b,c may couple to one or more springs to enable position adjustment of the needle assembly 215 (for example, to and from a tissue site), as disclosed herein. The structural component 2161 can include a slot 2164 that can engage the trigger pin 2113. FIGS. 22O-22T illustrate various views of the needle assembly 215. FIG. 22O shows the needle assembly 215 from a first view 215a. The needle assembly 215 can include an enclosure assembly 2151, a needle 217 for providing a cannula to a tissue site, and a cavity 2153 for receiving a support member (for example, to couple to the first support member 2162c). In some examples, the needle 217 can be curved and/or include a curved portion. FIG. 22P shows the needle assembly 215 from a second view 215b. FIG. 22Q shows the needle assembly 215 from a third view 215c. FIG. 22R shows the needle assembly 215 from a fourth view 215d. FIG. 22S shows the needle assembly 215 from a fifth view 215e. FIG. 22T shows the needle assembly 215 from a sixth view 215f.

In some examples, the injection system 210 (and components thereof) may be similar or identical to and/or incorporate any of the features described and/or illustrated with respect to any of the devices, assemblies, systems, and/or methods described and/or illustrated in U.S. application No. Ser. No. 18/605,630, filed Mar. 14, 2024 and titled “MODULAR DISEASE MANAGEMENT DEVICE AND AUTOMATED NEEDLE AND CANNULA INSERTION DEVICE,” the disclosure of which is hereby incorporated by reference in its entirety, and which claims priority to U.S. Provisional Application No. 63/490,601, which was filed on Mar. 16, 2023 and is titled “MODULAR DISEASE MANAGEMENT DEVICE AND AUTOMATED NEEDLE AND CANNULA INSERTION DEVICE,” the disclosure of which is expressly incorporated by reference herein in its entirety for all purposes.

### Needle Insertion

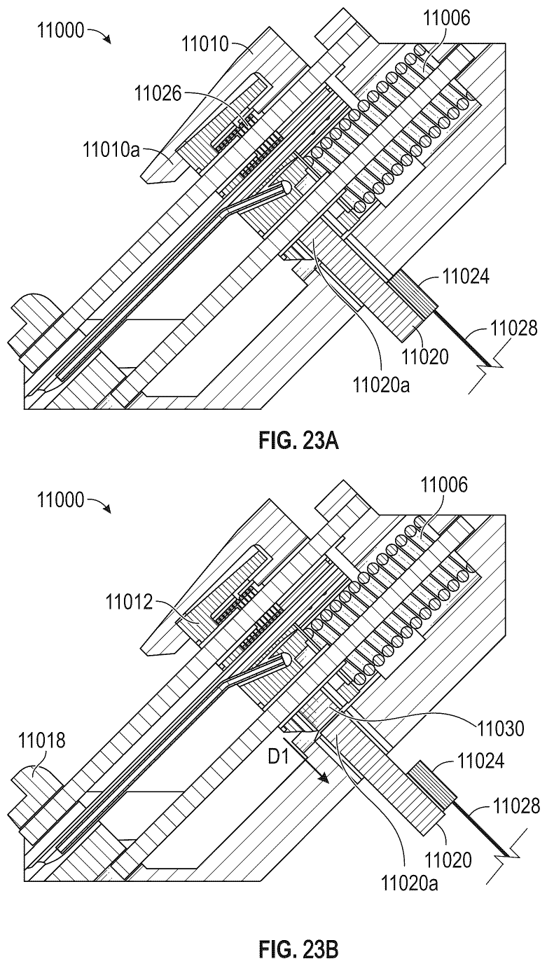

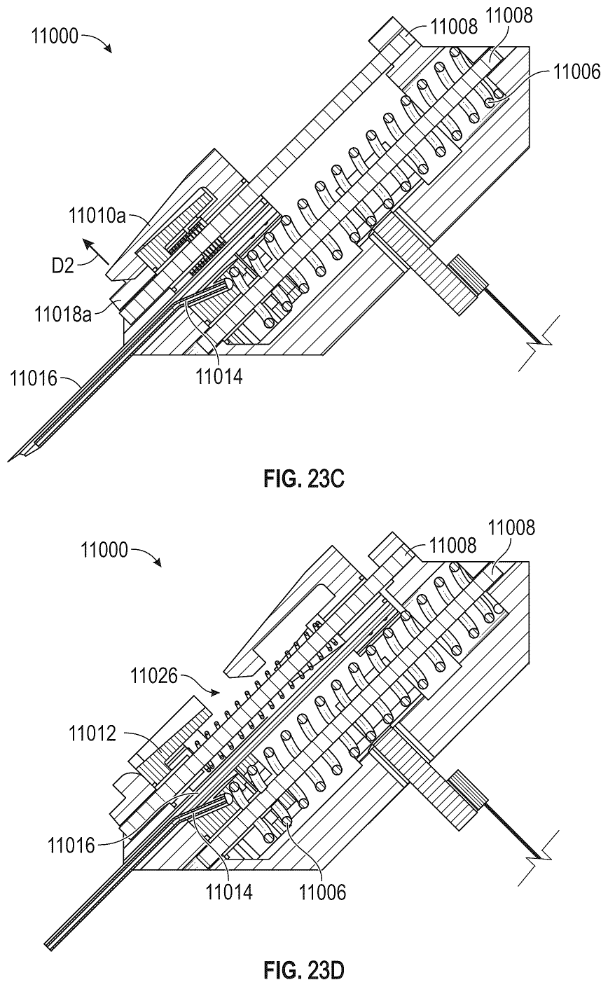

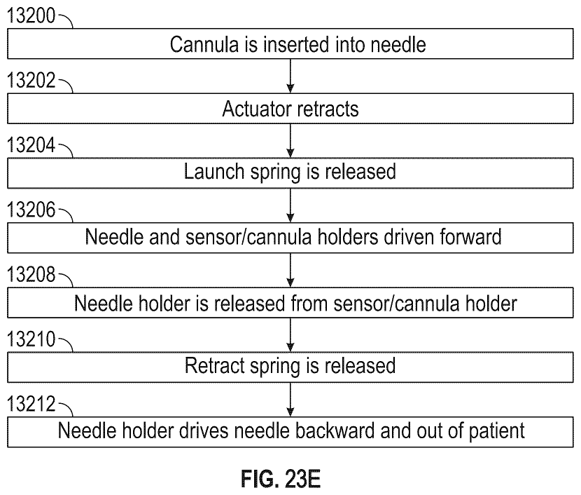

FIGS. 23A-23E illustrate an automated insertion and needle removal device at various positions while inserting a needle into and removing the needle from an insertion site of a patient to implant a cannula. The automated insertion and needle removal device depicted in FIGS. 23A-23E can be the same as or similar to the pump assembly 221 discussed with reference to FIGS. 22A-22T. Further, all of the components described with respect to FIGS. 22A-22T may be present in FIGS. 23A-23D and vice versa. FIG. 23A illustrates a cross section side view of an automated needle insertion and needle removal device prior to launch (also referred to as a prelaunch configuration). FIG. 23B illustrates a cross section side view of an automated needle insertion and needle removal device with a muscle wire actuated. FIG. 23C illustrates a cross section side view of an automated needle insertion and needle removal device with a needle in an inserted position. FIG. 23D illustrates a cross section side view of an automated needle insertion and needle removal device with a needle in a retracted position.

Referring to FIG. 23A, the launch spring 11006 and the retract spring 11026 are in a compressed state. The launch spring 11006 may bias the needle carrier 11010 towards a deployed position. The launch spring 11006 is held in place by a cannula carrier 11012 and the trigger pin 11020 at a first end 11020a. The retract spring 11026 is held in place by the needle carrier 11010. The needle carrier can include a latched portion 11010a (also referred to herein as a trigger pin) that may release the 11010 from the cannula carrier, thereby releasing the retract spring 11026. The trigger pin 11020 is coupled to a guide component 11024, which is coupled to a muscle wire 11028 at a first end. The second end of the muscle wire 11028 can be anchored to a static portion of the 100, as discussed with respect to the muscle wire 218 with reference to FIGS. 22A-22T.

Referring to FIG. 23B, as the muscle wire 11028 is actuated and contracts, force is applied on the trigger pin 11020, moving the trigger pin 11020 in direction D1. As the trigger pin 11020 moves in the direction D1, the first end 11020a may disengage the slot 11030 of cannula carrier 11012, thereby releasing the launch spring 11006.

Referring to FIG. 23C, when the launch spring 11006 is released, the launch spring 11006 may push the cannula carrier 11012 and the needle carrier 11010 forward along the guide rails 11008, such that the needle 11016 and the cannula 11014 are inserted into an insertion site of a patient. As illustrated in FIG. 23D, the launch spring 11006 may maintain tension as to hold the cannula carrier 11012 in place. When the automated insertion device 11000 is in the fully inserted position illustrated in FIG. 23C, the latched portion 11010a of the needle carrier 11010 may interact with a protruded portion 11018a of the body 11018, forcing the latched portion 11010a in direction D2. As the latched portion 11010a moves in direction D2, the needle carrier 11010 is released from the cannula carrier 11012, thereby releasing the retract spring 11026.

Referring to FIG. 23D, when the retract spring 11026 is released, the retract spring may push the needle carrier 11010 backward along the guide rails 11008, thereby removing the needle 11016 from the insertion site of the patient. The retract spring 11026 may also maintain tension such that the retract spring 11026 holds the needle carrier 11010 in a position and the needle 11016 remains fully retracted from the patient. As illustrated in FIG. 23D, the retract spring 11026 and the launch spring 11006 may secure the cannula carrier 11012 in place such that the cannula 11014 remains fixed in position in the insertion site of the patient.

FIG. 23E illustrates an example process for an automated needle insertion and needle removal device, such as automated insertion device 11000, for inserting a cannula into an insertion site of a patient. At block 13200 a cannula is inserted into a needle. In some implementations, a cannula, such as needle 11016 is inserted into needle 11016.

At block 13202, an actuator retracts. In some implementations, the actuator may be a retracting wire (e.g., a muscle wire) and retract using the process described above with respect to FIG. 23A. In some implementation, the actuator may retract in response to a physical force applied to the actuator.

At block 13204, a launch spring, such as launch spring 11006, is released. As described above, the actuator may be coupled to a release mechanism, for example the trigger pin 11020. When the actuator retracts, the release mechanism may disengage a sensor holder, a cannula holder, or other component, such that the launch spring is released.

At block 13206, the needle and sensor/cannula holders are driven forward. When the launch spring is released, the launch spring can push the needle holder and the sensor/cannula holder forward along the guide rails 11008. The needle holder may be coupled to a needle, such as 11016, and the sensor/cannula holder may be coupled to a cannula (e.g., cannula 11014). As such, as the needle holder and the sensor/cannula holder are driven forward, a needle and a sensor/cannula are also driven forward and inserted into an insertion site of the patient.

At block 13208, the needle holder is released from the sensor or cannula holder. For example, as illustrated in FIG. 23C, a protruded portion 11018a of the body 11018 may engage the needle carrier 11010, releasing the needled carrier from the cannula carrier 11012. At block 13210, the retract spring is released. As described above, the retract spring may have one end engaged with the needle carrier and another end engaged with the cannula/sensor holder. As such, when the needler holder is released at block 13208, the retract spring is free to extend.

At block 13212, the needle holder drives needle backward out of the insertion site of the patient. As the retract spring decompresses and extends, the retract spring can force the needle holder backward along the guide rails. Since the needle holder is coupled to the needle, as the needle holder is forced backward, the needle is also forced backward, thereby removing the needle for the insertion site. As described above, after the needle holder drives the needle backward, the retract spring and the launch spring maintain tension, holding the sensor/cannula in place in the insertion site and the needle in place out of the insertion site.

### Disease Management Device Housing

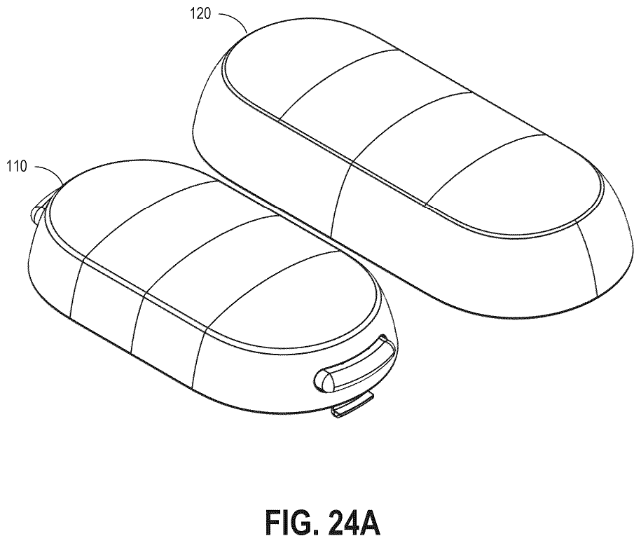

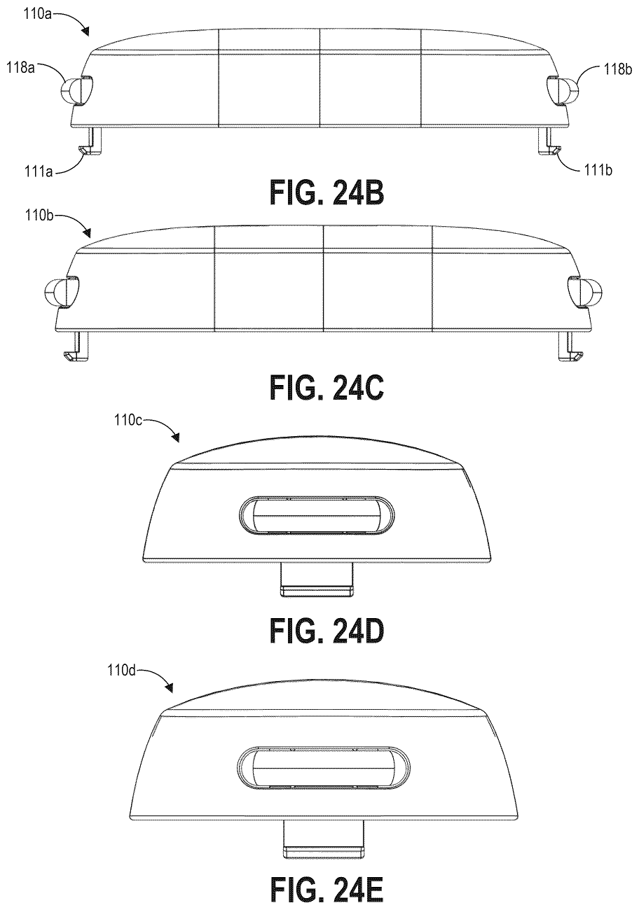

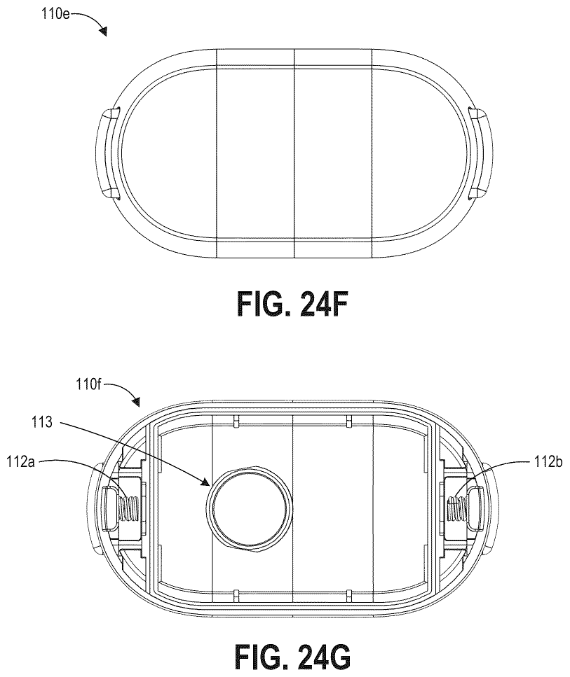

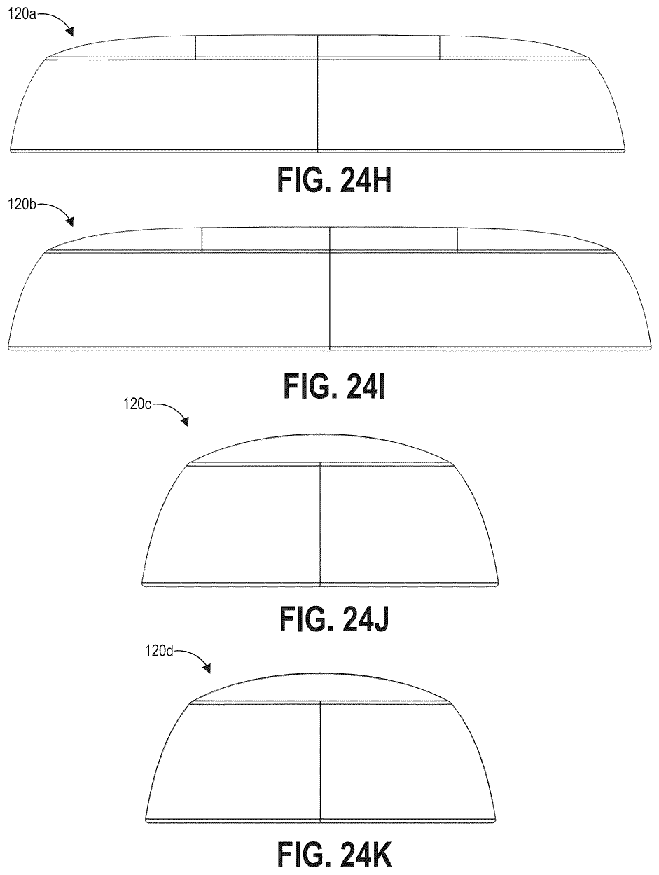

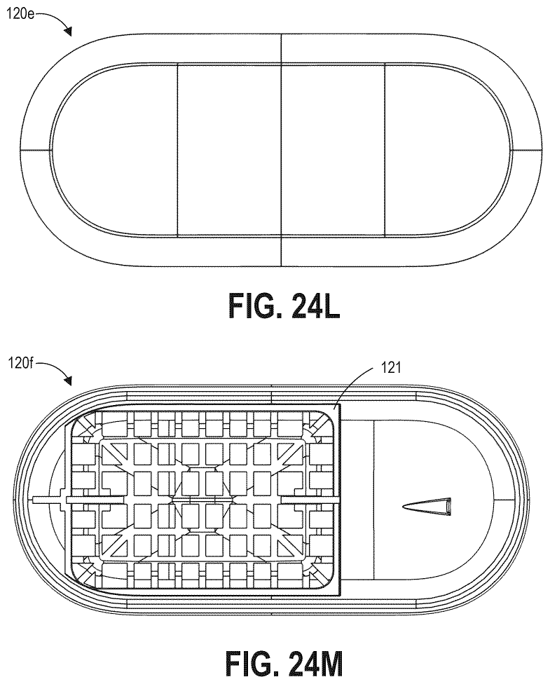

With reference to an illustrative example, FIG. 24A shows a perspective view of the first cover 110 of the first portion 103 and the second cover 120 of the second portion 104. The first portion 103 may be reusable. The second portion 104 and/or the first portion base 130 may be disposable. In some examples, the first portion 103 can reversibly couple to the base 130. For instance, the first cover 110 may engage using one or more retractable components 111a and 111b and spring components 112a and 112b (as illustrated in FIG. 24G) of the device 100 (such as illustrated in FIG. 1K-1) to attach to the first device hardware 101 and/or the base 130 (such as illustrated in FIG. 1K-1). For example, the first cover 110 may include openings, each opening able to receive one of the buttons 118a and 118b. In this way, the buttons 118a and 118b and the retractable components 111a and 111b may couple to one of the spring components 112a and 112b. As the retractable components 111a and 111b adjust a position, the spring components 112a 112b may activate (for example, compress or decompress). In this way, the retractable components 111a 111b may disengage (or engage) with the openings of the first cover 110 to allow the first cover 110 to separate from (or attach to) the first device hardware 101. Buttons 118a 118b can be pressed to actuate the one or more retractable components 111a 111b to allow for the first cover 110 to be released from the base 130. The first cover 110 may include a battery component 113 and other hardware components, such as electronics (not shown). In some examples, the second cover 120 may include the same (or substantially similar) capabilities and components as the first cover 110.

FIGS. 24B-24G illustrate various views of the first cover 110. FIG. 24B shows the first cover 110 from a first view 110a. FIG. 24C shows the first cover 110 from a second view 110b. FIG. 24D shows the first cover 110 from a third view 110c. FIG. 24E shows the first cover 110 from a fourth view 110d. FIG. 24F shows the first cover 110 from a fifth view 110e. FIG. 24G shows the first cover 110 from a sixth view 110f.

FIGS. 24H-24M illustrate various views of the second cover 120. FIG. 24H shows the second cover 120 from a first view 120a. FIG. 24I shows the second cover 120 from a second view 120b. FIG. 24J shows the second cover 120 from a third view 120c. FIG. 24K shows the second cover 120 from a fourth view 120d. FIG. 24L shows the second cover 120 from a fifth view 120e. FIG. 24M shows the second cover 120 from a sixth view 120f. The second cover 120 may include an assembly 121 along an interior of the second cover 120.

### Terminology and Additional Considerations

All of the methods and jobs described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and jobs may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design conditions imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method (including computer-implemented method), process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or”means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C. Unless otherwise explicitly stated, the terms “set” and “collection” should generally be interpreted to include one or more described items throughout this application. Accordingly, phrases such as “a set of devices configured to” or “a collection of devices configured to” are intended to include one or more recited devices. Such one or more recited devices can also be collectively configured to carry out the stated recitations. For example, “a set of servers configured to carry out recitations A, B and C” can include a first server configured to carry out recitation A working in conjunction with a second server configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A medication delivery pump system for delivering fluid medication to a user, the system comprising:
a pump, the pump comprising:
a first actuator;
a second actuator, each of the first actuator and second actuator configured to manipulate a medication volume passing through a medication flow path of the medication delivery pump system, each of the first actuator and second actuator comprising:
a plunger;
a disc spring, the disc spring opposing actuation of the plunger;
a shape memory alloy (SMA) wire configured to drive movement of the plunger, wherein the positions of the plunger fall within an inclusive range between an actuated and an unactuated position in the medication flow path; and
an electrical sensor configured to measure an electrical property of the SMA actuator;
one or more environmental sensors; and
a processor operably coupled to the electrical sensors, the SMA wires, and the one or more environmental sensors, the processor configured to execute computer readable instructions to:
receive a first electrical property from the electrical sensor of the first actuator and a second electrical property from the electrical sensor of the second actuator;
receive one or more signals from the one or more environmental sensors; and
control the first actuator and the second actuator, based at least in part on the first electrical property, the second electrical property, and the one or more signals from the one or more environmental sensors, to drive the movement of the first actuator and the second actuator to positions within the medication flow path correlated with a target medication dispense volume.

2. The medication delivery pump system of claim 1, wherein the one or more environmental sensors comprise at least one of a temperature sensor, a pressure sensor, or a humidity sensor.

3. The medication delivery pump system of claim 2, wherein the one or more environmental sensors comprise a temperature sensor, the temperature sensor positioned spaced from skin of a user when the medication delivery pump system is positioned on skin of a user.

4. The medication delivery pump system of claim 1, wherein the SMA wire comprises a muscle wire.

5. The medication delivery pump system of claim 4, wherein the muscle wire is configured to contract when electrical current is applied.

6. The medication delivery pump system of claim 1, wherein the processor is further configured to execute computer readable instructions to cease heating of the SMA wires of the first actuator or the second actuator, causing the first actuator or second actuator to close.

7. The medication delivery pump system of claim 1, comprising a base, the base comprising an adhesive at least partially coupled to a tissue site of a patient.

8. The medication delivery pump system of claim 7, wherein the delivery pump system comprises a reusable portion, the reusable portion comprising a first housing and the processor, the reusable portion configured to be reversibly coupled to the base.

9. The medication delivery pump system of claim 8, wherein the delivery pump system comprises a disposable portion, the disposable portion comprising a second housing, the base, and the pump.

10. The medication delivery pump system of claim 1, comprising a medication bladder in fluid communication with the pump.

11. The medication delivery pump system of claim 1, comprising a cannula in fluid communication with the pump.

12. The medication delivery pump system of claim 11, comprising a cannula insertion device.

13. The medication delivery pump system of claim 1, wherein movement of the plunger and disc spring exerts pressure on the medication flow path.

14. The medication delivery pump system of claim 1, wherein the plunger comprises a ridge, the ridge configured to create a seal with an interior cavity of the pump, wherein movement of the position of the seal exerts pressure on the medication flow path.

15. A method of dispensing a fluid from a medication pump system comprising a first shape memory alloy (SMA) actuator and a second SMA actuator positioned in series along a flow path, the method comprising:
sensing an environmental condition using one or more environmental sensors, wherein a processor is configured to receive one or more signals from the one or more environmental sensors;
determining, using the processor, a first target electrical property value for the first SMA actuator and a second target electrical property value for the second SMA actuator, wherein the first target electrical property value corresponds to a first target displacement of the first SMA actuator and the second target electrical property value corresponds to a second target displacement of the second SMA actuator, wherein the first target electrical property value and the second target electrical property value are determined based at least in part on the received one or more signals from the one or more environmental sensors, and wherein the processor is configured to receive a first electrical property measurement from the first SMA actuator and a second electrical property measurement from the second SMA actuator;
heating the first SMA actuator until the first electrical property measurement reaches the first target electrical property value, causing a first plunger of the first SMA actuator to be displaced; and
heating the second SMA actuator until the second electrical property measurement reaches the second target electrical property value, causing a second
plunger of the second SMA actuator to be displaced.

16. The method of claim 15, further comprising ceasing heating of the first SMA actuator, causing the first plunger to return to an unactuated position, and ceasing heating of the second SMA actuator, causing the second plunger return to an unactuated position.

17. The method of claim 15, wherein causing the first plunger to close comprises allowing a first spring to return the first plunger to the unactuated position, and causing the second plunger to close comprises allowing a second spring to return the second plunger to the unactuated position.

18. The method of claim 15, wherein heating the first SMA actuator comprises applying a high PWM duty cycle current to the first SMA actuator, and heating the second SMA actuator comprises applying a high PWM duty cycle current to the second SMA actuator.

19. The method of claim 15, further comprising maintaining a thermal steady state of the first SMA actuator, causing the first plunger to remain in an actuated position, and maintaining a thermal steady state of the second SMA actuator, causing the second plunger to remain in an actuated position.

20. The method of claim 15, wherein the environmental condition comprises one or more of an ambient temperature, an ambient pressure, or an ambient humidity.
